SEQUENCE LISTING (7 sequences)

SEQ ID NO: 1 - DNA, 3366 bp, Artificial Sequence
  Chemically Synthesized, synthetic consensus dTERT nucleotide sequence
cctcgggctcctcgctgtagggctgtgcgggctctgctgagaggccggtacagggaggtgctgccactggccaccttcctgaggagactgggccctcccggcagactgctggtgcggcgcggcgaccctgcagcctttcgggccctggtggcacagtgcctggtgtgcgtgccatggggcgcccgcccaccccctgcagcaccatgctttaggcaggtgagctgtctgaaggagctggtggcccgcgtggtgcagaggctgtgcgagagaggcgccaggaacgtgctggccttcggctttgccctgctggatggagcccgcggcggcccacccgtggccttcaccacaagcgtgaggtcctacctgcctaataccgtgacagagacactgagaggctccggagcatggggcctgctgctgaggagagtgggcgacgatgtgctgacacacctgctggcaaggtgcgcactgtatctgctggtggcaccatcctgcgcataccaggtgtgcggccctccactgtatgacctgtgcgcccctgcctctctgcccctgcctgccccaggcctgcctggcctgccaggcctgccaggcctgggagcaggagcaggagccagcgccgatctgcgcccaaccaggcaggcacagaactccggagcacggcgcaggagaggctctccaggcagcggcgtgcccctggccaagaggcctcggcgctccgtggcatctgagccagagagaggcgcccaccggtccttccctcgcgcccagcagcctcccgtgagcgaggccccagccgtgacaccagcagtggcagcatccccagcagcctcttgggagggcggcccacccggcacccggcctaccacaccagcatggcacccataccctggaccacagggagtgccacacgaccctgcccacccagagacaaagaggttcctgtattgtagcggcggcagggagagactgcggcctagctttctgctgtccgccctgcctccaacactgtctggcgccaggaagctggtggagacaatctttctgggcagcgcccctcagaagcctggagcagcaaggagaatgcggcgcctgcctgccagatactggcgcatgaggccactgttccaggagctgctgggaaaccacgcaaggtgcccttatagagccctgctgcggacacactgtccactgagagccatggccgccaaggagggctctggcaatcaggcccacagaggcgtgggaatctgccctctggagcggccagtggcagcccctcaggagcagacagatagcaccaggctggtgcagctgctgaggcagcacagctccccatggcaggtgtacgccttcctgagggcatgcctgtgctggctggtgccaaccggcctgtggggctccagacacaaccagaggagattcctgcggaatgtgaagaagtttatcagcctgggcaagcacgccaagctgtccctgcaggagctgacatggaagatgaaggtgagagactgtacctggctgcacggcaacccaggcgcctgctgcgtgccagcagcagagcaccggcgcagggaggagatcctggcaaggttcctggtgctggtggatggccacatctacgtggtgaagctgctgagatccttcttttatgtgaccgagactacgttccagaagaactacctgttcttttatcggaagagcgtgtggtcccagctgcagtctatcggcatccgccagctgttcaattctgtgcacctgagggagctgagcgaggcagaggtgagacggcacagagaggcccggccagccctgctgacatctagactgcggttcctgccagcacctagcggcctggcccccatcgtgaacatggactacatcatgggcgcccgcaccttccacagggataagaaggtgcagcacctgacatcccagctgaagaccctgttttctgtgctgaattatgagagggcacgcaggccttccctgctgggagcctctatgctgggcatggacgatatccaccgcgcctggaggacattcgtgctgcgcatcagggcacagaacccagcccctcagctgtactttgtgaaggtggcagtgaccggagcagcagacgcactgccccaggatcgcctggtggaagtgatcgccaatgtgatcagacctcaggagagcacatactgcgtgcggcactatgcagtggtgcagagaaccgccaggggccacgtgcgcaaggccttcaagaggcacgtgtccacatttgccgacctgcagccatatatgagacagtttgtggagcggctgcaggagacaagcctgctgagggatgccgtggtcatcgagcagtctagctccctgaacgaggccggctctagcctgttccacctgtttctgcgcctggtgcacaatcacgtggtgaggatcggcggcaagtcttacatccagtgtcagggcgtgccccagggctctatcctgagcaccctgctgtgcagcctgtgctatggcgacatggagagacggctgttccctggcatcgagcaggacggcgtgctgctgagactggtggccgatttcctgctggtgacaccacacctgacccaggcccaggcctttctgcggacactggtgaagggagtgcctgagtacggatgcagggcaaacctgcagaagaccgccgtgaatttcccagtggaggatggcgccctgggcagcgccgcccctctgcagctgccagcccactgcctgtttccatggtgtggcctgctgctggacacacggaccctggaggtgtcctgtgattactcctcttatgcccacacatctatcagggcaagcctgaccttttcccagggagcaaagccaggaaggaacatgcgcaggaagctgctggccgtgctgaggctgaagtgctgtgccctgttcctgtacctgcaggtgaacggcatccacacagtgtacatgaacgtgtacaagatcttcctgctgcaggcctatcggtttcacgcctgcgtgctgcagctgcccttcaaccagcctgtgagaaagaatcctagcttctttctgagagtgatcgccgacaccgcctcttgctgttacagcctgctgaaggcccgcaatgcaggcctgtccctgggagcaaagggagcaagcggcctgttcccatccgaggcagcaaggtggctgtgcctgcacgcctttctgctgaagctggcccaccacagcggcacatatagatgtctgctgggcgccctgcaggcagcaaaggcacacctgtccagacagctgccccggggcaccctggcagccctggaggcagccgcagacccttcactgacagccgacttcaagacaatcctggac

SEQ ID NO: 2 - protein, 1122 aa, Artificial Sequence
  Chemically Synthesized, synthetic consensus dTERT amino acid sequence
PRAPRCRAVRALLRGRYREVLPLATFLRRLGPPGRLLVRRGDPAAFRALVAQCLVCVPWGARPPPAAPCFRQVSCLKELVARVVQRLCERGARNVLAFGFALLDGARGGPPVAFTTSVRSYLPNTVTETLRGSGAWGLLLRRVGDDVLTHLLARCALYLLVAPSCAYQVCGPPLYDLCAPASLPLPAPGLPGLPGLPGLGAGAGASADLRPTRQAQNSGARRRRGSPGSGVPLAKRPRRSVASEPERGAHRSFPRAQQPPVSEAPAVTPAVAASPAASWEGGPPGTRPTTPAWHPYPGPQGVPHDPAHPETKRFLYCSGGRERLRPSFLLSALPPTLSGARKLVETIFLGSAPQKPGAARRMRRLPARYWRMRPLFQELLGNHARCPYRALLRTHCPLRAMAAKEGSGNQAHRGVGICPLERPVAAPQEQTDSTRLVQLLRQHSSPWQVYAFLRACLCWLVPTGLWGSRHNQRRFLRNVKKFISLGKHAKLSLQELTWKMKVRDCTWLHGNPGACCVPAAEHRRREEILARFLVLVDGHIYVVKLLRSFFYVTETTFQKNYLFFYRKSVWSQLQSIGIRQLFNSVHLRELSEAEVRRHREARPALLTSRLRFLPAPSGLAPIVNMDYIMGARTFHRDKKVQHLTSQLKTLFSVLNYERARRPSLLGASMLGMDDIHRAWRTFVLRIRAQNPAPQLYFVKVAVTGAADALPQDRLVEVIANVIRPQESTYCVRHYAVVQRTARGHVRKAFKRHVSTFADLQPYMRQFVERLQETSLLRDAVVIEQSSSLNEAGSSLFHLFLRLVHNHVVRIGGKSYIQCQGVPQGSILSTLLCSLCYGDMERRLFPGIEQDGVLLRLVADFLLVTPHLTQAQAFLRTLVKGVPEYGCRANLQKTAVNFPVEDGALGSAAPLQLPAHCLFPWCGLLLDTRTLEVSCDYSSYAHTSIRASLTFSQGAKPGRNMRRKLLAVLRLKCCALFLYLQVNGIHTVYMNVYKIFLLQAYRFHACVLQLPFNQPVRKNPSFFLRVIADTASCCYSLLKARNAGLSLGAKGASGLFPSEAARWLCLHAFLLKLAHHSGTYRCLLGALQAAKAHLSRQLPRGTLAALEAAADPSLTADFKTILD

SEQ ID NO: 3 - DNA, 3420 bp, Artificial Sequence
  Chemically Synthesized, synthetic consensus dTERT nucleotide sequence operably linked to an IgE leader sequence
atggactggacttggattctgttcctggtcgctgccgccactcgcgtgcattcacctcgggctcctcgctgtagggctgtgcgggctctgctgagaggccggtacagggaggtgctgccactggccaccttcctgaggagactgggccctcccggcagactgctggtgcggcgcggcgaccctgcagcctttcgggccctggtggcacagtgcctggtgtgcgtgccatggggcgcccgcccaccccctgcagcaccatgctttaggcaggtgagctgtctgaaggagctggtggcccgcgtggtgcagaggctgtgcgagagaggcgccaggaacgtgctggccttcggctttgccctgctggatggagcccgcggcggcccacccgtggccttcaccacaagcgtgaggtcctacctgcctaataccgtgacagagacactgagaggctccggagcatggggcctgctgctgaggagagtgggcgacgatgtgctgacacacctgctggcaaggtgcgcactgtatctgctggtggcaccatcctgcgcataccaggtgtgcggccctccactgtatgacctgtgcgcccctgcctctctgcccctgcctgccccaggcctgcctggcctgccaggcctgccaggcctgggagcaggagcaggagccagcgccgatctgcgcccaaccaggcaggcacagaactccggagcacggcgcaggagaggctctccaggcagcggcgtgcccctggccaagaggcctcggcgctccgtggcatctgagccagagagaggcgcccaccggtccttccctcgcgcccagcagcctcccgtgagcgaggccccagccgtgacaccagcagtggcagcatccccagcagcctcttgggagggcggcccacccggcacccggcctaccacaccagcatggcacccataccctggaccacagggagtgccacacgaccctgcccacccagagacaaagaggttcctgtattgtagcggcggcagggagagactgcggcctagctttctgctgtccgccctgcctccaacactgtctggcgccaggaagctggtggagacaatctttctgggcagcgcccctcagaagcctggagcagcaaggagaatgcggcgcctgcctgccagatactggcgcatgaggccactgttccaggagctgctgggaaaccacgcaaggtgcccttatagagccctgctgcggacacactgtccactgagagccatggccgccaaggagggctctggcaatcaggcccacagaggcgtgggaatctgccctctggagcggccagtggcagcccctcaggagcagacagatagcaccaggctggtgcagctgctgaggcagcacagctccccatggcaggtgtacgccttcctgagggcatgcctgtgctggctggtgccaaccggcctgtggggctccagacacaaccagaggagattcctgcggaatgtgaagaagtttatcagcctgggcaagcacgccaagctgtccctgcaggagctgacatggaagatgaaggtgagagactgtacctggctgcacggcaacccaggcgcctgctgcgtgccagcagcagagcaccggcgcagggaggagatcctggcaaggttcctggtgctggtggatggccacatctacgtggtgaagctgctgagatccttcttttatgtgaccgagactacgttccagaagaactacctgttcttttatcggaagagcgtgtggtcccagctgcagtctatcggcatccgccagctgttcaattctgtgcacctgagggagctgagcgaggcagaggtgagacggcacagagaggcccggccagccctgctgacatctagactgcggttcctgccagcacctagcggcctggcccccatcgtgaacatggactacatcatgggcgcccgcaccttccacagggataagaaggtgcagcacctgacatcccagctgaagaccctgttttctgtgctgaattatgagagggcacgcaggccttccctgctgggagcctctatgctgggcatggacgatatccaccgcgcctggaggacattcgtgctgcgcatcagggcacagaacccagcccctcagctgtactttgtgaaggtggcagtgaccggagcagcagacgcactgccccaggatcgcctggtggaagtgatcgccaatgtgatcagacctcaggagagcacatactgcgtgcggcactatgcagtggtgcagagaaccgccaggggccacgtgcgcaaggccttcaagaggcacgtgtccacatttgccgacctgcagccatatatgagacagtttgtggagcggctgcaggagacaagcctgctgagggatgccgtggtcatcgagcagtctagctccctgaacgaggccggctctagcctgttccacctgtttctgcgcctggtgcacaatcacgtggtgaggatcggcggcaagtcttacatccagtgtcagggcgtgccccagggctctatcctgagcaccctgctgtgcagcctgtgctatggcgacatggagagacggctgttccctggcatcgagcaggacggcgtgctgctgagactggtggccgatttcctgctggtgacaccacacctgacccaggcccaggcctttctgcggacactggtgaagggagtgcctgagtacggatgcagggcaaacctgcagaagaccgccgtgaatttcccagtggaggatggcgccctgggcagcgccgcccctctgcagctgccagcccactgcctgtttccatggtgtggcctgctgctggacacacggaccctggaggtgtcctgtgattactcctcttatgcccacacatctatcagggcaagcctgaccttttcccagggagcaaagccaggaaggaacatgcgcaggaagctgctggccgtgctgaggctgaagtgctgtgccctgttcctgtacctgcaggtgaacggcatccacacagtgtacatgaacgtgtacaagatcttcctgctgcaggcctatcggtttcacgcctgcgtgctgcagctgcccttcaaccagcctgtgagaaagaatcctagcttctttctgagagtgatcgccgacaccgcctcttgctgttacagcctgctgaaggcccgcaatgcaggcctgtccctgggagcaaagggagcaagcggcctgttcccatccgaggcagcaaggtggctgtgcctgcacgcctttctgctgaagctggcccaccacagcggcacatatagatgtctgctgggcgccctgcaggcagcaaaggcacacctgtccagacagctgccccggggcaccctggcagccctggaggcagccgcagacccttcactgacagccgacttcaagacaatcctggac

SEQ ID NO: 4 - protein, 1140 aa, Artificial Sequence
  Chemically Synthesized, synthetic consensus dTERT amino acid sequence operably linked to an IgE leader sequence
MDWTWILFLVAAATRVHSPRAPRCRAVRALLRGRYREVLPLATFLRRLGPPGRLLVRRGDPAAFRALVAQCLVCVPWGARPPPAAPCFRQVSCLKELVARVVQRLCERGARNVLAFGFALLDGARGGPPVAFTTSVRSYLPNTVTETLRGSGAWGLLLRRVGDDVLTHLLARCALYLLVAPSCAYQVCGPPLYDLCAPASLPLPAPGLPGLPGLPGLGAGAGASADLRPTRQAQNSGARRRRGSPGSGVPLAKRPRRSVASEPERGAHRSFPRAQQPPVSEAPAVTPAVAASPAASWEGGPPGTRPTTPAWHPYPGPQGVPHDPAHPETKRFLYCSGGRERLRPSFLLSALPPTLSGARKLVETIFLGSAPQKPGAARRMRRLPARYWRMRPLFQELLGNHARCPYRALLRTHCPLRAMAAKEGSGNQAHRGVGICPLERPVAAPQEQTDSTRLVQLLRQHSSPWQVYAFLRACLCWLVPTGLWGSRHNQRRFLRNVKKFISLGKHAKLSLQELTWKMKVRDCTWLHGNPGACCVPAAEHRRREEILARFLVLVDGHIYVVKLLRSFFYVTETTFQKNYLFFYRKSVWSQLQSIGIRQLFNSVHLRELSEAEVRRHREARPALLTSRLRFLPAPSGLAPIVNMDYIMGARTFHRDKKVQHLTSQLKTLFSVLNYERARRPSLLGASMLGMDDIHRAWRTFVLRIRAQNPAPQLYFVKVAVTGAADALPQDRLVEVIANVIRPQESTYCVRHYAVVQRTARGHVRKAFKRHVSTFADLQPYMRQFVERLQETSLLRDAVVIEQSSSLNEAGSSLFHLFLRLVHNHVVRIGGKSYIQCQGVPQGSILSTLLCSLCYGDMERRLFPGIEQDGVLLRLVADFLLVTPHLTQAQAFLRTLVKGVPEYGCRANLQKTAVNFPVEDGALGSAAPLQLPAHCLFPWCGLLLDTRTLEVSCDYSSYAHTSIRASLTFSQGAKPGRNMRRKLLAVLRLKCCALFLYLQVNGIHTVYMNVYKIFLLQAYRFHACVLQLPFNQPVRKNPSFFLRVIADTASCCYSLLKARNAGLSLGAKGASGLFPSEAARWLCLHAFLLKLAHHSGTYRCLLGALQAAKAHLSRQLPRGTLAALEAAADPSLTADFKTILD

SEQ ID NO: 5 - protein, 15 aa, Artificial Sequence
  Chemically Synthesized, immunodominant epitope of synthetic consensus dTERT
FNSVHLRELSEAEVR

SEQ ID NO: 6 - DNA, 54 bp, Artificial Sequence
  Chemically Synthesized, nucleotide sequence
atggactggacttggattctgttcctggtcgctgccgccactcgcgtgcattca

SEQ ID NO: 7 - protein, 18 aa, Artificial Sequence
  Chemically Synthesized, amino acid sequence
MDWTWILFLVAAATRVHS

(19) United States
(12) Patent Application Publication
(10) Pub. No.: US 2020/0147195 A1
(43) Pub. Date: May 14, 2020

(54) DTERT VACCINES AND METHODS OF TREATMENT USING THE SAME

(71) Applicant: The Wistar Institute of Anatomy and Biology, Philadelphia, PA (US)

(72) Inventors: Kar Muthumani, Cherry Hill, NJ (US); David B. Weiner, Merion, PA (US)

(21) Appl. No.: 16/617,101

(22) PCT Filed: May 24, 2018
(86) PCT No.: PCT/US2018/034323

§ 371 Date: Nov. 26, 2019

Related U.S. Application Data
(60) Provisional application No. 62/511,594, filed on May 26, 2017.

(51) Int. Cl. A61K 39/00; A61K 39/395; A61P 35/00

(52) U.S. Cl. CPC A61K 39/001157; A61K 48/00; A61P 35/00; A61K 39/3955

(57) ABSTRACT

Disclosed herein are compositions and methods for treating and/or preventing cancer in dogs, and in particular, vaccines that treat and provide protection against tumor growth.

### CROSS REFERENCE TO RELATED APPLICATIONS

This application claims priority to U.S. Provisional Application No. 62/511,594, filed May 26, 2017 which is hereby incorporated by reference herein in its entirety.

### TECHNICAL FIELD

Disclosed herein are compositions and methods for treating cancer and in particular, vaccines that treat and provide protection against tumor growth.

### BACKGROUND

Telomerase is a ribonucleoprotein enzyme essential for the replication of chromosome termini in most eukaryotes, which regulates cell proliferation and immortality. In most types of cells, telomerase is either undetectable or active at very low levels, however telomerase is highly active in cells that divide rapidly. Further, increased telomerase activity is associated with malignancy. Cancer treatments involving inhibiting the catalytic component of telomerase (TERT), to reduce the TERT enzyme's activity can cause senescence and apoptosis without affecting normal human cells.

Transcription factors that can activate TERT include many oncogenes (cancer-causing genes) such as c-Myc, Sp1, HIF-1, AP2, among others, while many cancer suppressing genes such as p53, WT1, and Menin produce factors that suppress TERT activity.

TERT expression has been related to tumor progression as well as survival in malignancies, and may represent a broadly useful antigen for an attractive DNA immune therapy target in cancer. Cancer is not limited to any specific species, with Lymphoma among the most common cancers in dogs.

Accordingly, a need exists for the identification and development of compositions and methods for the prevention and/or treatment of cancer in dogs. Furthermore, more effective treatments are required to delay disease progression and/or decrease mortality in subjects suffering from cancer.

### SUMMARY OF INVENTION

In one aspect, the present invention provides a vaccine comprising a nucleotide sequence encoding a consensus dTERT antigen. In one embodiment the consensus dTERT antigen comprises an amino acid sequence selected from the group consisting of SEQ ID NO:2, SEQ ID NO:4, an amino acid sequence that is 95% identical or greater to SEQ ID NO:2 and an amino acid sequence that is 95% identical or greater to SEQ ID NO:4.

In one embodiment, the vaccine further comprises one or more nucleotide sequences encoding one or more additional cancer antigens. In one embodiment, the one or more additional cancer antigens comprise one or more antigens selected from the group consisting of the amino acid sequence of tyrosinase (Tyr), the amino acid sequence of tyrosinase-related protein 1 (TYRP1), the amino acid sequence of tyrosinase-related protein 2 (TYRP2), the amino acid sequence of melanoma-associated antigen 4 protein (MAGEA4), the amino acid sequence of growth hormone release hormone (GHRH), the amino acid sequence of MART-1/melan-A antigen (MART-1/Melan-A), the amino acid sequence of cancer testis antigen (NY-ESO-1), the amino acid sequence of cancer testis antigen II (NY-ESO-2), the amino acid sequence of PRAME, the amino acid sequence of WT1, the amino acid sequence of PSA, the amino acid sequence of PSMA, the amino acid sequence of STEAP, the amino acid sequence of PSCA, the amino acid sequence of MAGE A1, the amino acid sequence of gp100, the amino acid sequence of a viral antigen, and a combination thereof.

In one embodiment, the vaccine further comprises one or more nucleotide sequences encoding one or more immune checkpoint inhibitors. In one embodiment, the immune checkpoint inhibitor is selected from the group consisting of: anti-PD-1 antibody, anti-PD-L1 antibody, anti-TIM-3 antibody, anti-LAG-3 antibody, anti-CTLA4 antibody, and a combination thereof.

In one embodiment, the nucleotide sequence comprises a nucleotide sequence selected from the group consisting of: SEQ ID NO:1, SEQ ID NO:3, a nucleotide sequence that is 95% identical or greater to SEQ ID NO:1, and a nucleotide sequence that is 95% identical or greater to SEQ ID NO:3.

In one embodiment, the vaccine comprises one or more plasmids.

In one embodiment, the vaccine comprises a nucleotide sequence encoding an adjuvant. In one embodiment, the adjuvant is IL-12, IL-15, IL-28, or RANTES.

In one aspect, the present invention provides a method of treating cancer in a subject in need thereof. In one embodiment the method comprises administering a vaccine comprising a nucleotide sequence encoding a consensus dTERT antigen. In one embodiment the consensus dTERT antigen comprises an amino acid sequence selected from the group consisting of SEQ ID NO:2, SEQ ID NO:4, an amino acid sequence that is 95% identical or greater to SEQ ID NO:2 and an amino acid sequence that is 95% identical or greater to SEQ ID NO:4.

In one embodiment, the administering step comprises electroporation. In one embodiment, the method further comprises administering one or more nucleotide sequences encoding one or more immune checkpoint inhibitors. In one embodiment, the immune checkpoint inhibitor is selected from the group consisting of: anti-PD-1 antibody, anti-PD-L1 antibody, anti-TIM-3 antibody, anti-LAG-3 antibody, anti-CTLA4 antibody, and a combination thereof.

In one embodiment, the cancer is selected from the group consisting of: a blood cancer, melanoma, head and neck cancer, prostate cancer, liver cancer, cervical cancer, anal cancer, a papilloma and a combination thereof.

In one aspect, the present invention provides a nucleic acid molecule comprising one or more nucleotide sequences selected from the group consisting of: SEQ ID NO:1, SEQ ID NO:3, a nucleotide sequence that is 95% identical or greater to SEQ ID NO:1, and a nucleotide sequence that is 95% identical or greater to SEQ ID NO:3. In one embodiment, the nucleotide sequence comprises one or more plasmids.

In one aspect, the present invention provides a protein comprising one or more amino acid sequences selected from the group consisting of: SEQ ID NO:2, SEQ ID NO:4, an amino acid sequence that is 95% identical or greater to SEQ ID NO:2, and an amino acid sequence that is 95% identical or greater to SEQ ID NO:4.

### BRIEF DESCRIPTION OF THE DRAWINGS

FIG. 1, comprising FIG. 1A and FIG. 1B, depicts the construction of dTERT-PL. FIG. 1A depicts a plasmid diagram showing that a synthetic consensus canine (or dog) TERT (dTERT) was cloned into an expression plasmid to generate dTERT-PL. FIG. 1B depicts exemplary experimental data demonstrating that the synthetic consensus dTERT was inserted into the plasmid.

FIG. 2 depicts exemplary experimental results demonstrating that there was a high level of expression of dTERT in cells after transfection.

FIG. 3 depicts the immunization schedule used for immunization experiments.

FIG. 4, comprising FIG. 4A and FIG. 4B, depicts exemplary experimental results demonstrating a cellular immune response induced by Dog TERT (PL) DNA vaccine in mice. FIG. 4A depicts exemplary experimental results demonstrating the total dTERT-specific IFN-γ responses one week after the 3rd immunization with the dTERT vaccine (25 μg). FIG. 4B depicts exemplary experimental results demonstrating the dTERT-specific IFN-γ responses one week after 3rd immunization when splenocytes from each mouse (4 mice per group) were stimulated with dTERT peptide pools separately.

FIG. 5 depicts exemplary experimental results demonstrating that dTERT epitopes were assayed for their ability to induce a high level IFN-γ response in C57/BL6 mice.

FIG. 6, comprising FIG. 6A and FIG. 6B, depicts exemplary experimental results demonstrating a humoral immune response after immunization with DNA construct expressing dTERT. FIG. 6A depicts exemplary experimental results demonstrating total IgG antibody titers in the sera of immunized mice. FIG. 6A depicts exemplary experimental results demonstrating that specificity was detected by immunofluorescence in 293T cells transfected with DNA plasmid vaccine encoding the dTERT, treated with immune serum from the mice.

### DETAILED DESCRIPTION

The present invention is directed to a vaccine for use in treating cancers and tumors in canines. Antigen consensus sequences have been designed for the cancer related antigen TERT to be used in the vaccine to allow customized vaccine-mediated prevention and treatment of particular cancers. For example, dTERT antigen may be used in the vaccine for prevention or treatment of lymphomas. The vaccine of the invention can be used along with any combination of additional cancer antigens for the treatment or prevention of a cancer in a subject in need thereof.

One manner for designing the nucleic acid and its encoded amino acid sequence of the recombinant cancer antigen is by introducing mutations that change particular amino acids in the overall amino acid sequence of the native cancer antigen. The introduction of mutations does not alter the cancer antigen so much that it cannot be universally applied across a mammalian subject, for example, a dog subject, but changes it enough that the resulting amino acid sequence breaks tolerance or is considered a foreign antigen in order to generate an immune response. Another manner may be creating a consensus recombinant cancer antigen that has at least 85% and up to 99% amino acid sequence identity to its corresponding native cancer antigen; at least 90% and up to 98% sequence identity; at least 93% and up to 98% sequence identity; or at least 95% and up to 98% sequence identity. In some instances the recombinant cancer antigen has 95%, 96%, 97%, 98%, or 99% amino acid sequence identity to its corresponding native cancer antigen. The native cancer antigen is the antigen normally associated with the particular cancer or cancer tumor (e.g., native dTERT). Depending upon the cancer antigen, the consensus sequence of the cancer antigen can be across mammalian species or within subtypes of a species (e.g., across one or more species of canines). Some cancer antigens do not vary greatly from the wild type amino acid sequence of the cancer antigen. Some cancer antigens have nucleic acid/amino acid sequences that are so divergent across species, that a consensus sequence cannot be generated. In these instances, a recombinant cancer antigen that will break tolerance and generate an immune response is generated that has at least 85% and up to 99% amino acid sequence identity to its corresponding native cancer antigen; at least 90% and up to 98% sequence identity; at least 93% and up to 98% sequence identity; or at least 95% and up to 98% sequence identity. In some instances the recombinant cancer antigen has up to 95%, 96%, 97%, 98%, or 99% amino acid sequence identity to its corresponding native cancer antigen.

The recombinant cancer antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule.

The vaccine may be combined further with antibodies to checkpoint molecules, including but not limited to PD-1, PDL-1, TIM3, LAG3 and CTLA4 to increase the stimulation of both the cellular and humoral immune responses. Using anti-checkpoint molecule antibodies prevents the immune checkpoint from suppressing T-cell and/or B-cell responses. Overall, by designing the cancer antigens to be recognized by the immune system helps to overcome other forms of immune suppression by tumor cells, and these vaccines can be used in combination with suppression or inhibition therapies (such as anti-PD-1, anti-PDL-1, anti-TIM3, anti-LAG3 and anti-CTLA4 antibody therapies) to further increase T-cell and/or B-cell responses.

### 1. DEFINITIONS

Unless otherwise defined, all technical and scientific terms used herein have the same meaning as commonly understood by one of ordinary skill in the art. In case of conflict, the present document, including definitions, will control. Exemplary methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting. The terminology used herein is for the purpose of describing particular embodiments only and is not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“Adjuvant” as used herein means any molecule added to the DNA plasmid vaccines described herein to enhance the immunogenicity of the antigens encoded by the DNA plasmids and the encoding nucleic acid sequences described hereinafter.

“Antibody” as used herein means an antibody of classes IgG, IgM, IgA, IgD or IgE, or fragments, or derivatives thereof, including Fab, F(ab′)2, Fd, and single chain antibodies, diabodies, bispecific antibodies, bifunctional antibodies and derivatives thereof. The antibody can be an antibody isolated from the serum sample of mammal, a polyclonal antibody, affinity purified antibody, or mixtures thereof which exhibits sufficient binding specificity to a desired epitope or a sequence derived therefrom.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means a nucleic acid can mean Watson-Crick (e.g., A-T/U and C-G) or Hoogsteen base pairing between nucleotides or nucleotide analogs of nucleic acid molecules.

“Consensus” or “consensus sequence” as used herein means a polypeptide sequence based on analysis of an alignment of multiple sequences for the same gene from different organisms. Nucleic acid sequences that encode a consensus polypeptide sequence can be prepared. Vaccines comprising proteins that comprise consensus sequences and/or nucleic acid molecules that encode such proteins can be used to induce broad immunity against an antigen.

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Fragment” as used herein with respect to nucleic acid sequences means a nucleic acid sequence or a portion thereof, that encodes a polypeptide capable of eliciting an immune response in a mammal that cross reacts with an antigen disclosed herein. The fragments can be DNA fragments selected from at least one of the various nucleotide sequences that encode protein fragments set forth below. Fragments can comprise at least 10%, at least 20%, at least 30%, at least 40%, at least 50%, at least 60%, at least 70%, at least 80%, at least 90%, or at least 95% of one or more of the nucleic acid sequences set forth below. In some embodiments, fragments can comprise at least 20 nucleotides or more, at least 30 nucleotides or more, at least 40 nucleotides or more, at least 50 nucleotides or more, at least 60 nucleotides or more, at least 70 nucleotides or more, at least 80 nucleotides or more, at least 90 nucleotides or more, at least 100 nucleotides or more, at least 150 nucleotides or more, at least 200 nucleotides or more, at least 250 nucleotides or more, at least 300 nucleotides or more, at least 350 nucleotides or more, at least 400 nucleotides or more, at least 450 nucleotides or more, at least 500 nucleotides or more, at least 550 nucleotides or more, at least 600 nucleotides or more, at least 650 nucleotides or more, at least 700 nucleotides or more, at least 750 nucleotides or more, at least 800 nucleotides or more, at least 850 nucleotides or more, at least 900 nucleotides or more, at least 950 nucleotides or more, or at least 1000 nucleotides or more of at least one of the nucleic acid sequences set forth below.

“Fragment” or “immunogenic fragment” with respect to polypeptide sequences means a polypeptide capable of eliciting an immune response in a mammal that cross reacts with an antigen disclosed herein. The fragments can be polypeptide fragments selected from at least one of the various amino acids sequences below. Fragments of consensus proteins can comprise at least 10%, at least 20%, at least 30%, at least 40%, at least 50%, at least 60%, at least 70%, at least 80%, at least 90% or at least 95% of a consensus protein. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more of a protein sequence disclosed herein.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operably linked to a coding sequence that encodes a protein such that when present in the cell of the individual, the coding sequence will be expressed.

The term “homology,” as used herein, refers to a degree of complementarity. There can be partial homology or complete homology (i.e., identity). A partially complementary sequence that at least partially inhibits a completely complementary sequence from hybridizing to a target nucleic acid is referred to using the functional term “substantially homologous.” When used in reference to a double-stranded nucleic acid sequence such as a cDNA or genomic clone, the term “substantially homologous,” as used herein, refers to a probe that can hybridize to a strand of the double-stranded nucleic acid sequence under conditions of low stringency. When used in reference to a single-stranded nucleic acid sequence, the term “substantially homologous,” as used herein, refers to a probe that can hybridize to (i.e., is the complement of) the single-stranded nucleic acid template sequence under conditions of low stringency.

“Identical” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host's immune system, e.g., that of a mammal, in response to the introduction of antigen. The immune response can be in the form of a cellular or humoral response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively, or differentially with respect to the cell, tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein may facilitate secretion of the protein from the cell in which it is produced. Signal peptides/leader sequences are often cleaved from the remainder of the protein, often referred to as the mature protein, upon secretion from the cell. Signal peptides/leader sequences are linked at the amino terminus (i.e., N terminus) of the protein.

“Stringent hybridization conditions” as used herein means conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions can be selected to be about 5-10° C. lower than the thermal melting point (Tm) for the specific sequence at a defined ionic strength pH. The Tm can be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions can be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions can also be achieved with the addition of destabilizing agents such as formamide. For selective or specific hybridization, a positive signal can be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” as used herein can mean a mammal that is capable of being immunized with the vaccines described herein. The mammal can be, for example, a human, chimpanzee, dog, cat, horse, cow, mouse, or rat.

“Substantially complementary” as used herein means that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

“Substantially identical” as used herein means that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

“Treatment” or “treating” as used herein can mean protecting an animal from a disease through means of preventing, suppressing, repressing, or completely eliminating the disease. Preventing the disease involves administering a vaccine of the present invention to an animal prior to onset of the disease. Suppressing the disease involves administering a vaccine of the present invention to an animal after induction of the disease but before its clinical appearance. Repressing the disease involves administering a vaccine of the present invention to an animal after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

“Variant” with respect to a peptide or polypeptide refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art, for example, see Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions can be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full-length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the gene sequence or a fragment thereof. A variant may be an amino acid sequence that is substantially identical over the full-length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full-length of the amino acid sequence or a fragment thereof.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and for example, may be a DNA plasmid. The vector can contain or include one or more heterologous nucleic acid sequences.

### 2. VACCINE

The present invention is directed to an anti-cancer vaccine. The vaccine can comprise dTERT alone or in combination with one or more cancer antigens. The vaccine can prevent tumor growth. The vaccine can reduce tumor growth. The vaccine can prevent metastasis of tumor cells. The vaccine can be targeted to treat blood cancers, liver cancer, prostate cancer, melanomas, head and neck cancer, glioblastoma, recurrent respiratory papillomatosis, anal cancer, cervical cancer, and brain cancer.

In one embodiment, the vaccine comprises a synthetic consensus dTERT antigen that is recognized by the immune system and breaks tolerance to a self-antigen. The dTERT antigen identified is modified from a self-antigen in order to be recognized by the immune system as a foreign antigen, while retaining sufficient similarity to the self-antigen to promote an immune response against the self-antigen. The redesign of the nucleic acid and amino acid sequence of the recombinant cancer antigen from a self to a foreign antigen breaks tolerance of antigen by the immune system. In order to break tolerance, several redesign measures can be applied to the cancer antigen as described below.

The synthetic consensus dTERT antigen of the vaccine is not recognized as self, and therefore can break tolerance. The breaking of tolerance can induce antigen-specific T cell and/or high titer antibody responses, thereby inducing or eliciting an immune response that is directed to or reactive against the cancer or tumor expressing native dTERT. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-3, tumor associated macrophages, tumor associated fibroblasts, soluble factors produced by immune suppressor cells, CTLA-4, PD-1, MDSCs, MCP-1, and an immune checkpoint molecule.

In a particular embodiment, the vaccine can mediate clearance or prevent growth of tumor cells by inducing (1) humoral immunity via B cell responses to generate antibodies that block monocyte chemoattractant protein-1 (MCP-1) production, thereby retarding myeloid derived suppressor cells (MDSCs) and suppressing tumor growth; (2) increase cytotoxic T lymphocyte such as CD8⁺ (CTL) to attack and kill tumor cells; (3) increase T helper cell responses; (4) and increase inflammatory responses via IFN-γ and TNF-α or all of the aforementioned. The vaccine can increase tumor free survival by at least 1%, 5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43% 44%, 45%, or more than 45% relative to tumor free survival in the absence of the vaccine. The vaccine can reduce tumor mass by at least 1%, 5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60% or more than 60% after immunization relative to the tumor mass prior to immunization. The vaccine can prevent and block increases in monocyte chemoattractant protein 1 (MCP-1), a cytokine secreted by myeloid derived suppressor cells. The vaccine can increase survival by at least 1%, 5%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60% or more than 60% after immunization relative to survival in the absence of the vaccine.

The vaccine can increase a cellular immune response in a subject administered the vaccine by about 2-fold to about 6000-fold, about 3-fold to about 6000-fold, about 4-fold to about 6000-fold, about 5-fold to about 6000-fold, about 6-fold to about 6000-fold, about 7-fold to about 6000-fold, about 8-fold to about 6000-fold, about 9-fold to about 6000-fold, about 10-fold to about 6000-fold, about 15-fold to about 6000-fold, about 10-fold to about 6000-fold, about 25-fold to about 6000-fold, about 30-fold to about 6000-fold, about 35-fold to about 6000-fold, about 40-fold to about 6000-fold, about 45-fold to about 6000-fold, about 50-fold to about 6000-fold, about 2-fold to about 5500-fold, about 2-fold to about 5000-fold, about 2-fold to about 4500-fold, about 100-fold to about 6000-fold, about 150-fold to about 6000-fold, about 200-fold to about 6000-fold, about 250-fold to about 6000-fold, or about 300-fold to about 6000-fold as compared to a cellular immune response in a subject not administered the vaccine. In some embodiments the vaccine can increase the cellular immune response in the subject administered the vaccine by about 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, 1000-fold, 1100-fold, 1200-fold, 1300-fold, 1400-fold, 1500-fold, 1600-fold, 1700-fold, 1800-fold, 1900-fold, 2000-fold, 2100-fold, 2200-fold, 2300-fold, 2400-fold, 2500-fold, 2600-fold, 2700-fold, 2800-fold, 2900-fold, 3000-fold, 3100-fold, 3200-fold, 3300-fold, 3400-fold, 3500-fold, 3600-fold, 3700-fold, 3800-fold, 3900-fold, 4000-fold, 4100-fold, 4200-fold, 4300-fold, 4400-fold, 4500-fold, 4600-fold, 4700-fold, 4800-fold, 4900-fold, 5000-fold, 5100-fold, 5200-fold, 5300-fold, 5400-fold, 5500-fold, 5600-fold, 5700-fold, 5800-fold, 5900-fold, or 6000-fold as compared to the cellular immune response in the subject not administered the vaccine.

The vaccine can increase interferon gamma (IFN-γ) levels in a subject administered the vaccine by about 2-fold to about 6000-fold, about 3-fold to about 6000-fold, about 4-fold to about 6000-fold, about 5-fold to about 6000-fold, about 6-fold to about 6000-fold, about 7-fold to about 6000-fold, about 8-fold to about 6000-fold, about 9-fold to about 6000-fold, about 10-fold to about 6000-fold, about 15-fold to about 6000-fold, about 10-fold to about 6000-fold, about 25-fold to about 6000-fold, about 30-fold to about 6000-fold, about 35-fold to about 6000-fold, about 40-fold to about 6000-fold, about 45-fold to about 6000-fold, 50-fold to about 6000-fold, about 2-fold to about 5500-fold, about 2-fold to about 5000-fold, about 2-fold to about 4500-fold, about 100-fold to about 6000-fold, about 150-fold to about 6000-fold, about 200-fold to about 6000-fold, about 250-fold to about 6000-fold, or about 300-fold to about 6000-fold as compared to IFN-γ levels in a subject not administered the vaccine. In some embodiments the vaccine can increase IFN-γ levels in the subject administered the vaccine by about 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, 1000-fold, 1100-fold, 1200-fold, 1300-fold, 1400-fold, 1500-fold, 1600-fold, 1700-fold, 1800-fold, 1900-fold, 2000-fold, 2100-fold, 2200-fold, 2300-fold, 2400-fold, 2500-fold, 2600-fold, 2700-fold, 2800-fold, 2900-fold, 3000-fold, 3100-fold, 3200-fold, 3300-fold, 3400-fold, 3500-fold, 3600-fold, 3700-fold, 3800-fold, 3900-fold, 4000-fold, 4100-fold, 4200-fold, 4300-fold, 4400-fold, 4500-fold, 4600-fold, 4700-fold, 4800-fold, 4900-fold, 5000-fold, 5100-fold, 5200-fold, 5300-fold, 5400-fold, 5500-fold, 5600-fold, 5700-fold, 5800-fold, 5900-fold, or 6000-fold as compared to IFN-γ levels in the subject not administered the vaccine.

The vaccine can be a DNA vaccine. DNA vaccines are disclosed in U.S. Pat. Nos. 5,593,972, 5,739,118, 5,817,637, 5,830,876, 5,962,428, 5,981,505, 5,580,859, 5,703,055, and 5,676,594, which are incorporated herein fully by reference. The DNA vaccine can further comprise elements or reagents that inhibit it from integrating into the chromosome.

The vaccine can be an RNA of the one or more cancer antigens. The RNA vaccine can be introduced into the cell.

The vaccine can be an attenuated live vaccine, a vaccine using recombinant vectors to deliver antigen, subunit vaccines, and glycoprotein vaccines, for example, but not limited, the vaccines described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference.

The vaccine of the present invention can have features required of effective vaccines such as being safe so that the vaccine itself does not cause illness or death; being protective against illness; inducing neutralizing antibody; inducing protective T cell responses; and providing ease of administration, few side effects, biological stability, and low cost per dose. The vaccine can accomplish some or all of these features by comprising the cancer antigen as discussed below.

As described in more detail below, the vaccine can further comprise one or more inhibitors of one or more immune checkpoint molecules (i.e., an immune checkpoint inhibitor). Immune checkpoint molecules are described below in more detail. The immune checkpoint inhibitor may be any nucleic acid or protein that prevents the suppression of any component in the immune system such as MHC class presentation, T cell presentation and/or differentiation, B cell presentation and/or differentiation, any cytokine, chemokine or signaling for immune cell proliferation and/or differentiation. As also described below in more detail, the vaccine may be combined further with one or more antibodies to checkpoint molecules such as PD-1, PDL-1, TIM-3, LAG-3 and CTLA4 to increase the stimulation of both the cellular and humoral immune responses. Using anti-checkpoint molecule antibodies prevents immune checkpoint proteins from suppressing T-cell and/or B-cell responses.

The synthetic consensus antigen can be a nucleic acid sequence, an amino acid sequence, or a combination thereof. The at least one cancer antigen can be a nucleic acid sequence, an amino acid sequence, or a combination thereof. The nucleic acid sequence can be DNA, RNA, cDNA, a variant thereof, a fragment thereof, or a combination thereof. The nucleic acid sequence can also include additional sequences that encode linker or tag sequences that are linked to the antigen by a peptide bond. The amino acid sequence can be a protein, a peptide, a variant thereof, a fragment thereof, or a combination thereof. The cancer antigen can be a recombinant cancer antigen.

One manner for designing the nucleic acid and its encoded amino acid sequence of the recombinant cancer antigen is by introducing mutations that change particular amino acids in the overall amino acid sequence of the native cancer antigen. The introduction of mutations does not alter the cancer antigen so much that it cannot be universally applied across a mammalian subject, and preferably a human or dog subject, but changes it enough that the resulting amino acid sequence breaks tolerance or is considered a foreign antigen in order to generate an immune response. Another manner may be creating a consensus recombinant cancer antigen that has at least 85% and up to 99% amino acid sequence identity to its corresponding native cancer antigen; at least 90% and up to 98% sequence identity; at least 93% and up to 98% sequence identity; or at least 95% and up to 98% sequence identity. In some instances the recombinant cancer antigen is 95%, 96%, 97%, 98%, or 99% amino acid sequence identity to its corresponding native cancer antigen. The native cancer antigen is the antigen normally associated with the particular cancer or cancer tumor. Depending upon the cancer antigen, the consensus sequence of the cancer antigen can be across mammalian species or within subtypes of a species or across viral strains or serotypes. Some cancer antigens do not vary greatly from the wild type amino acid sequence of the cancer antigen. Some cancer antigens have nucleic acid/amino acid sequences that are so divergent across species, that a consensus sequence cannot be generated. In these instances, a recombinant cancer antigen that will break tolerance and generate an immune response is generated that has at least 85% and up to 99% amino acid sequence identity to its corresponding native cancer antigen; preferably at least 90% and up to 98% sequence identity; more preferably at least 93% and up to 98% sequence identity; or even more preferably at least 95% and up to 98% sequence identity. In some instances the recombinant cancer antigen is 95%, 96%, 97%, 98%, or 99% amino acid sequence identity to its corresponding native cancer antigen. The aforementioned approaches can be combined so that the final recombinant cancer antigen has a percent similarity to native cancer antigen amino acid sequence as discussed, above.

a. dTERT Antigen

The vaccine of the present invention can comprise the cancer antigen dTERT, a fragment thereof, or a variant thereof. dTERT is the catalytic subunit of telomerase reverse transcriptase that synthesizes a TTAGGG tag on the end of telomeres to prevent cell death due to chromosomal shortening. Hyperproliferative cells can have abnormally high expression of dTERT. Abnormal expression of dTERT can also occur in hyperproliferative cells infected with viruses (e.g., canine papillomaviruses and canine hepacivirus). Thus, immunotherapy for viruses may be enhanced by targeting cells that express dTERT at abnormal levels. Viral antigens are discussed below in more detail.

Additionally, TERT expression in dendritic cells transfected with dTERT genes can induce CD8⁺ cytotoxic T cells and elicit CD4⁺ T cells in an antigen-specific fashion. Therefore, use of dTERT expression within antigen presenting cells (APCs) to delay senescence and sustain their capacity to present the antigen of choice can be used in immunotherapeutic methods such as in the methods described herein.

The dTERT antigen can be associated with or expressed by any number of cancers including, but not limited to, blood cancers, melanoma, prostate cancer, liver cancer, cervical cancer, papillomas, anal cancer, and head and neck cancer. Accordingly, the vaccine, when including the dTERT antigen described herein, can be used for treating subjects suffering from any number of cancers including, but not limited to, blood cancers, melanoma, prostate cancer, liver cancer, cervical cancer, papillomas, anal cancer, and head and neck cancer.

The dTERT antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The dTERT antigen can comprise protein epitopes that make them particularly effective as immunogens against which anti-dTERT immune responses can be induced. The dTERT antigen can comprise the full-length translation product, a variant thereof, a fragment thereof or a combination thereof. The dTERT antigen can comprise a consensus protein.

The nucleic acid sequence encoding the dTERT antigen or consensus dTERT antigen can be optimized with regards to codon usage and corresponding RNA transcripts. The nucleic acid encoding the dTERT antigen or consensus dTERT antigen can be codon and RNA optimized for expression in canines. In some embodiments, the nucleic acid sequence encoding the dTERT antigen or consensus dTERT antigen can include a Kozak sequence to increase the efficiency of translation. The nucleic acid encoding the dTERT antigen or consensus dTERT antigen can include or be operably linked to one or multiple stop codons (e.g., encoded by a sequence such as TGA or TGATAA) to increase the efficiency of translation termination.

The nucleic acid encoding the dTERT antigen or consensus dTERT antigen can also encode an immunoglobulin E (IgE) leader sequence. The nucleic acid encoding the dTERT antigen or consensus dTERT antigen can further encode the IgE leader sequence such that the amino acid sequence of the IgE leader sequence is linked to the amino acid sequence of the dTERT antigen or consensus dTERT antigen by a peptide bond, respectively. In one embodiment, an amino acid sequence of a consensus dTERT antigen operably linked to an IgE leader sequence is set forth in SEQ ID NO:4. The nucleic acid encoding the dTERT antigen or consensus dTERT antigen can also include a nucleotide sequence encoding the IgE leader sequence. In one embodiment, a nucleotide sequence encoding a consensus dTERT antigen operably linked to a sequence encoding an IgE leader sequence is set forth in SEQ ID NO:3. In some embodiments, the nucleic acid encoding the dTERT antigen or consensus dTERT antigen is free of or does not contain a nucleotide sequence encoding the IgE leader sequence. In one embodiment, a nucleotide sequence encoding a consensus dTERT antigen that does not contain a nucleotide sequence encoding the IgE leader sequence is set forth in SEQ ID NO:1 and encodes a dTERT antigen as set forth in SEQ ID NO:2.

In one embodiment, the nucleotide sequence encoding any component of a composition of the present invention may comprise an RNA sequence. For example, in one embodiment, the nucleotide sequence comprises an RNA sequence transcribed by the DNA sequence of SEQ ID NOs: 1, 3, or a variant thereof or a fragment thereof. In another embodiment, the nucleotide sequence comprises an RNA sequence transcribed by a DNA sequence encoding the polypeptide sequence of SEQ ID NOs: 2, 4, 5, or a variant thereof or a fragment thereof.

In some embodiments, the nucleic acid encoding the dTERT antigen or consensus dTERT antigen can be a heterologous nucleic acid sequence and/or contain one or more heterologous nucleic acid sequences. The nucleic acid encoding the dTERT antigen or consensus dTERT antigen can be mutated relative to the wild-type dTERT antigen such that one or more amino acids or residues in the amino acid sequence of the dTERT antigen or consensus dTERT antigen, respectively, is replaced or substituted with another amino acid or residue. The nucleic acid encoding the dTERT antigen or consensus dTERT antigen can be mutated relative to the wild-type dTERT antigen such that one or more residues in the amino acid sequence of the dTERT antigen or consensus dTERT antigen, respectively, are replaced or substituted with another residue, thereby causing the immune system to no longer be tolerant of dTERT in the mammal administered the nucleic acid encoding the dTERT antigen or consensus dTERT antigen, the dTERT antigen or consensus dTERT antigen, or combinations thereof.

In one aspect, the dTERT antigen can be the nucleic acid sequence SEQ ID NO:1, which encodes for the amino acid sequence SEQ ID NO:2. SEQ ID NO:3 encodes the dTERT protein linked to an IgE leader sequence. The dTERT protein can be linked to the IgE leader sequence and an HA tag. In other embodiments, the dTERT protein can be free of or not linked to an IgE leader sequence and/or an HA tag.

In some embodiments, the dTERT antigen can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in the SEQ ID NO:1. In some embodiments, the dTERT antigen can be an RNA encoded by a DNA sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in the SEQ ID NO:1. In some embodiments, the dTERT antigen can be an RNA that encodes an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2. In other embodiments, the dTERT antigen can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in one of SEQ ID NO:2. The dTERT antigen can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:2.

In some embodiments, the dTERT antigen can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in the SEQ ID NO:3. In some embodiments, the dTERT antigen can be an RNA encoded by a DNA sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in the SEQ ID NO:3. In some embodiments, the dTERT antigen can be an RNA that encodes an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:4. In other embodiments, the dTERT antigen can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in one of SEQ ID NO:4. The dTERT antigen can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:4.

Some embodiments relate to nucleic acid sequences encoding proteins homologous to the dTERT protein, an immunogenic fragment of the dTERT protein, and immunogenic fragments of homologous proteins. Such nucleic acid molecules that encode immunogenic proteins that have at least 95% homology to a sequence, at least 96% homology to a sequence, at least 97% homology to a sequence, at least 98% homology to a sequence and at least 99% can be provided. Likewise, nucleic acid sequences encoding the immunogenic fragments set forth herein and the immunogenic fragments of proteins homologous to the proteins set forth herein are also provided.

Some embodiments relate to nucleic acid molecules that encode immunogenic proteins that have at least 95% homology to the nucleic acid coding sequences herein. Some embodiments relate to nucleic acid molecules that encode immunogenic proteins that have at least 96% homology to the nucleic acid coding sequences herein. Some embodiments relate to nucleic acid molecules that encode immunogenic proteins that have at least 97% homology to the nucleic acid coding sequences herein. Some embodiments relate to nucleic acid molecules that encode immunogenic proteins that have at least 98% homology to the nucleic acid coding sequences herein. Some embodiments relate to nucleic acid molecules that encode immunogenic proteins that have at least 99% homology to the nucleic acid coding sequences herein. In some embodiments, the nucleic acid molecules with coding sequences disclosed herein that are homologous to a coding sequence of a consensus protein disclosed herein include sequences encoding an IgE leader sequence linked to the 5′ end of the coding sequence encoding the homologous protein sequences disclosed herein.

Some embodiments relate to nucleic acid sequences encoding proteins with a particular percent identity to the full-length dTERT protein, immunogenic fragment of the dTERT protein, and immunogenic fragments of proteins having identity to the dTERT protein. Such nucleic acid molecules that encode immunogenic proteins that have at least 80% identity to a full-length dTERT sequence, at least 85% identity to a full-length sequence, at least 90% identity to a full-length dTERT sequence, at least 91% identity to a full-length dTERT sequence, at least 92% identity to a full-length dTERT sequence, at least 93% identity to a full-length dTERT sequence, at least 94% identity to a full-length dTERT sequence, at least 95% identity to a full-length dTERT sequence, at least 96% identity to a full-length dTERT sequence, at least 97% identity to a full-length dTERT sequence, at least 98% identity to a full-length dTERT sequence, and at least 99% identity to a full-length dTERT sequence can be provided. Likewise, nucleic acid sequences encoding the immunogenic fragments set forth herein and the immunogenic fragments of proteins with similar percent identities as indicated above to the dTERT antigens set forth herein are also provided.

In some embodiments, the nucleic acid sequence is free of coding sequence that encodes a leader sequence. In some embodiments, the nucleic acid sequence is free of coding sequence that encodes the IgE leader.

Some embodiments relate to fragments of SEQ ID NO:1. Fragments can be at least 10%, at least 15%, at least 20%, at least 25%, at least 30%, at least 35%, at least 40%, at least 45%, at least 50%, at least 55% at least 60%, at least 65%, at least 70%, at least 75%, at least 80%, at least 85%, at least 90%, at least 95%, at least 96%, at least 97%, at least 98% or at least 99% of SEQ ID NO:1. Fragments can be at least 95%, at least 96%, at least 97%, at least 98%, or at least 99% homologous to fragments of SEQ ID NO:1. Fragments can be at least 80%, at least 85%, at least 90% at least 91%, at least 92%, at least 93%, at least 94%, at least 95%, at least 96%, at least 97%, at least 98%, or at least 99% identical to fragments of SEQ ID NO:1. In some embodiments, fragments include sequences that encode a leader sequence, such as for example, an immunoglobulin leader, such as the IgE leader. In some embodiments, fragments are free of coding sequences that encode a leader sequence. In some embodiments, fragments are free of coding sequences that encode a leader sequence, such as for example, the IgE leader.

Furthermore, the amino acid sequence of the dTERT protein is SEQ ID NO:2. The amino acid sequence of the dTERT protein linked to an IgE leader is SEQ ID NO:4. The amino acid sequence of the dTERT protein linked to the IgE leader may be linked to an HA tag.

Some embodiments relate to proteins that are homologous to SEQ ID NO:2. Some embodiments relate to immunogenic proteins that have at least 95% homology to the protein sequences as set forth in SEQ ID NO:2. Some embodiments relate to immunogenic proteins that have at least 96% homology to the protein sequences as set forth in SEQ ID NO:2. Some embodiments relate to immunogenic proteins that have at least 97% homology to the protein sequences as set forth in SEQ ID NO:2. Some embodiments relate to immunogenic proteins that have at least 98% homology to the protein sequences as set forth in SEQ ID NO:2. Some embodiments relate to immunogenic proteins that have at least 99% homology to the protein sequences as set forth in SEQ ID NO:2.

Some embodiments relate to proteins that are homologous to SEQ ID NO:4. Some embodiments relate to immunogenic proteins that have at least 95% homology to the protein sequences as set forth in SEQ ID NO:4. Some embodiments relate to immunogenic proteins that have at least 96% homology to the protein sequences as set forth in SEQ ID NO:4. Some embodiments relate to immunogenic proteins that have at least 97% homology to the protein sequences as set forth in SEQ ID NO:4. Some embodiments relate to immunogenic proteins that have at least 98% homology to the protein sequences as set forth in SEQ ID NO:4. Some embodiments relate to immunogenic proteins that have at least 99% homology to the protein sequences as set forth in SEQ ID NO:4.

Some embodiments relate to proteins that are identical to SEQ ID NO:2. Some embodiments relate to immunogenic proteins that have an amino acid sequence that is at least 80% identical to the full-length amino acid sequences as set forth in SEQ ID NO:2. Some embodiments relate to immunogenic proteins that have an amino acid sequence that is at least 85% identical to the full-length amino acid sequences as set forth in SEQ ID NO:2. Some embodiments relate to immunogenic proteins that have an amino acid sequence that is at least 90% identical to the full-length amino acid sequences as set forth in SEQ ID NO:2. Some embodiments relate to immunogenic proteins that have an amino acid sequence that is at least 91% identical to the full-length amino acid sequences as set forth in SEQ ID NO:2. Some embodiments relate to immunogenic proteins that have an amino acid sequence that is at least 92% identical to the full-length amino acid sequences as set forth in SEQ ID NO:2. Some embodiments relate to immunogenic proteins that have an amino acid sequence that is at least 93% identical to the full-length amino acid sequences as set forth in SEQ ID NO:2. Some embodiments relate to immunogenic proteins that have an amino acid sequence that is at least 94% identical to the full-length amino acid sequences as set forth in SEQ ID NO:2. Some embodiments relate to immunogenic proteins that have an amino acid sequence that is at least 95% identical to the full-length amino acid sequences as set forth in SEQ ID NO:2. Some embodiments relate to immunogenic proteins that have an amino acid sequence that is at least 96% identical to the full-length amino acid sequences as set forth in SEQ ID NO:2. Some embodiments relate to immunogenic proteins that have an amino acid sequence that is at least 97% identical to the full-length amino acid sequences as set forth in SEQ ID NO:2. Some embodiments relate to immunogenic proteins that have an amino acid sequence that is at least 98% identical to the full-length amino acid sequences as set forth in SEQ ID NO:2. Some embodiments relate to immunogenic proteins that have an amino acid sequence that is at least 99% identical to the full-length amino acid sequences as set forth in SEQ ID NO:2.

In some embodiments, the protein is free of a leader sequence. In some embodiments, the protein is free of the IgE leader.

Fragments of proteins can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% of a protein. Immunogenic fragments of SEQ ID NO:2 can be provided. Immunogenic fragments can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% of SEQ ID NO:2. In some embodiments, fragments include a leader sequence, such as for example, an immunoglobulin leader, such as the IgE leader. In some embodiments, fragments are free of a leader sequence. In some embodiments, fragments are free of a leader sequence, such as for example, the IgE leader. In one embodiment, an immunogenic fragment comprises an immunodominant or subdominant epitope of dTERT. In one embodiment, an immunodominant epitope comprises an amino acid sequence as set forth in SEQ ID NO:5.

Immunogenic fragments of proteins with amino acid sequences homologous to immunogenic fragments of SEQ ID NO:2 can be provided. Such immunogenic fragments can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% of proteins that are 95% or greater homologous to SEQ ID NO:2. Some embodiments relate to immunogenic fragments that have at least 96% homology to the immunogenic fragments of protein sequences herein. Some embodiments relate to immunogenic fragments that have at least 97% homology to the immunogenic fragments of protein sequences herein. Some embodiments relate to immunogenic fragments that have at least 98% homology to the immunogenic fragments of protein sequences herein. Some embodiments relate to immunogenic fragments that have at least 99% homology to the immunogenic fragments of protein sequences herein. In some embodiments, fragments include a leader sequence, such as for example, an immunoglobulin leader, such as the IgE leader. In some embodiments, fragments are free of a leader sequence. In some embodiments, fragments are free of a leader sequence, such as for example, the IgE leader.

Immunogenic fragments of proteins with amino acid sequences that are at least 80%, 85%, 90%, 91%, 92%, 93%, 94%, 95%, 96%, 97%, 98%, or 99% identical to immunogenic fragments of SEQ ID NO:2 can be provided. Such immunogenic fragments can comprise at least 10%, at least 15%, at least 20%, at least 25%, at least 30%, at least 35%, at least 40%, at least 45%, at least 50%, at least 55% at least 60%, at least 65%, at least 70%, at least 75%, at least 80%, at least 85%, at least 90%, at least 95%, at least 96%, at least 97%, at least 98% or at least 99% of proteins that are at least 80%, 85%, 90%, 91%, 92%, 93%, 94%, 95%, 96%, 97%, 98%, or 99% identical to the amino acid sequence set forth in SEQ ID NO:2. In some embodiments, fragments include a leader sequence, such as for example, an immunoglobulin leader, such as the IgE leader. In some embodiments, fragments are free of a leader sequence. In some embodiments, fragments are free of a leader sequence, such as for example, the IgE leader.

As referred to herein with regard to linking a signal peptide or leader sequence to the N terminus of a protein, in one embodiment, the signal peptide/leader sequence comprises an N terminal methionine of a protein. In one embodiment, an N-terminal methionine is encoded by a start codon. In one embodiment, a start codon is operably linked to the 5′ end of a nucleic acid sequence that encodes the protein.

Fragments of SEQ ID NO:1 may comprise at least 30, 45, 60, 75, 90, 120, 150, 180, 210, 240, 270, 300, 360, 420, 480, 540, 600, 660, 720, 780, 840, 900, 960, 1020, 1080, 1140, 1200, 1260, 1320, 1380, 1440, 1500, 1560, 1620, 1680, 1740, 1800, 1860, 1920, 1980, 2040, 2100, 2160, 2220, 2280, 2340, 2400, 2460, 2520, 2580, 2640, 2700, 2760, 2820, 2880, 2940, 3000, 3060, 3120, 3180, 3240, 3300, 3360 or more nucleotides of SEQ ID NO:1. In one embodiment, fragments of SEQ ID NO:1 comprise sequences that encode an immunodominant epitope. In one embodiment, an immunodominant epitope is set forth in SEQ ID NO:5. In one embodiment, a fragment of SEQ ID NO:1 comprises a sequence that encodes SEQ ID NO:5. In some embodiments, fragments of SEQ ID NO:2 may comprise coding sequences for the IgE leader sequences. In some embodiments, fragments of SEQ ID NO:2 do not comprise coding sequences for the IgE leader sequences.

Fragments of SEQ ID NO:1 may comprise fewer than 60, 75, 90, 120, 150, 180, 210, 240, 270, 300, 360, 420, 480, 540, 600, 660, 720, 780, 840, 900, 960, 1020, 1080, 1140, 1200, 1260, 1320, 1380, 1440, 1500, 1560, 1620, 1680, 1740, 1800, 1860, 1920, 1980, 2040, 2100, 2160, 2220, 2280, 2340, 2400, 2460, 2520, 2580, 2640, 2700, 2760, 2820, 2880, 2940, 3000, 3060, 3120, 3180, 3240, 3300, or fewer than 3360 nucleotides of SEQ ID NO:1.

Fragments of SEQ ID NO:2 may comprise at least 15, 18, 21, 24, 30, 36, 42, 48, 54, 60, 72, 90, 120, 150, 180, 210, 240, 270, 300, 330, 360, 390, 420, 450, 480, 510, 540, 570, 600, 630, 660, 690, 720, 750, 780, 810, 840, 870, 900, 930, 960, 990, 1020, 1050, 1080, 1110 or more amino acids of SEQ ID NO:2. In some embodiments, fragments of SEQ ID NO:2 comprise an immunodominant epitope. In one embodiment, fragments of SEQ ID NO:2 comprise SEQ ID NO:5. In some embodiments, fragments of SEQ ID NO:2 may comprise coding sequences for the IgE leader sequences. In some embodiments, fragments of SEQ ID NO:2 do not comprise coding sequences for the IgE leader sequences.

Fragments of SEQ ID NO:2 may comprise fewer than 24, 30, 36, 42, 48, 54, 60, 72, 90, 120, 150, 180, 210, 240, 270, 300, 330, 360, 390, 420, 450, 480, 510, 540, 570, 600, 630, 660, 690, 720, 750, 780, 810, 840, 870, 900, 930, 960, 990, 1020, 1050, 1080, or fewer than 1110 amino acids of SEQ ID NO:2.

In one embodiment, the consensus dTERT antigen is a synthetic consensus dTERT. In certain embodiments, the synthetic consensus dTERT comprises 1 or more, 2 or more, 3 or more, 4 or more, 5 or more, 6 or more, 7 or more, 8 or more 9 or more, 10 or more, 15 or more, 20 or more, 30 or more, or 50 or more amino acid mutations relative to the wild-type dTERT. For example, the consensus dTERT antigen can be the nucleic acid sequence SEQ ID NO:3, which encodes for the amino acid sequence SEQ ID NO:4. SEQ ID NO:3 encodes the consensus dTERT protein linked to an IgE leader sequence. The consensus dTERT protein can be linked to the IgE leader sequence and an HA tag. In other embodiments, the consensus dTERT protein can be free of or not linked to an IgE leader sequence and/or an HA tag.

In some embodiments, the consensus dTERT antigen can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence set forth in the SEQ ID NO:3. In other embodiments, the consensus dTERT antigen can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:4. The consensus dTERT antigen can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence set forth in SEQ ID NO:4.

Some embodiments relate to nucleic acid sequences encoding proteins homologous to the consensus dTERT protein, immunogenic fragment of the consensus dTERT protein, and immunogenic fragments of homologous proteins. Such nucleic acid molecules that encode immunogenic proteins that have at least 95% homology to a sequence, at least 96% homology to a sequence, at least 97% homology to a sequence, at least 98% homology to a sequence and at least 99% can be provided. Likewise, nucleic acid sequences encoding the immunogenic fragments set forth herein and the immunogenic fragments of proteins homologous to the proteins set forth herein are also provided.

Some embodiments relate to nucleic acid molecules that encode immunogenic proteins that have at least 95% homology to the nucleic acid coding sequences herein. Some embodiments relate to nucleic acid molecules that encode immunogenic proteins that have at least 96% homology to the nucleic acid coding sequences herein. Some embodiments relate to nucleic acid molecules that encode immunogenic proteins that have at least 97% homology to the nucleic acid coding sequences herein. Some embodiments relate to nucleic acid molecules that encode immunogenic proteins that have at least 98% homology to the nucleic acid coding sequences herein. Some embodiments relate to nucleic acid molecules that encode immunogenic proteins that have at least 99% homology to the nucleic acid coding sequences herein. In some embodiments, the nucleic acid molecules with coding sequences disclosed herein that are homologous to a coding sequence of a consensus protein disclosed herein include sequences encoding an IgE leader sequence linked to the 5′ end of the coding sequence encoding the homologous protein sequences disclosed herein.

Some embodiments relate to nucleic acid sequences encoding proteins with a particular percent identity to the full-length consensus dTERT protein, immunogenic fragment of the consensus dTERT protein, and immunogenic fragments of proteins having identity to the consensus dTERT protein. Such nucleic acid molecules that encode immunogenic proteins that have up to 80% identity to a full-length consensus dTERT sequence, up to 85% identity to a full-length sequence, up to 90% identity to a full-length consensus dTERT sequence, up to 91% identity to a full-length consensus dTERT sequence, up to 92% identity to a full-length consensus dTERT sequence, up to 93% identity to a full-length consensus dTERT sequence, up to 94% identity to a full-length consensus dTERT sequence, up to 95% identity to a full-length consensus dTERT sequence, up to 96% identity to a full-length consensus dTERT sequence, up to 97% identity to a full-length consensus dTERT sequence, up to 98% identity to a full-length consensus dTERT sequence, and up to 99% identity to a full-length consensus dTERT sequence can be provided. Likewise, nucleic acid sequences encoding the immunogenic fragments set forth herein and the immunogenic fragments of proteins with similar percent identities as indicated above to the consensus dTERT proteins set forth herein are also provided.

In some embodiments, the nucleic acid sequence is free of coding sequence that encodes a leader sequence. In some embodiments, the nucleic acid sequence is free of coding sequence that encodes the IgE leader.

Some embodiments relate to fragments of SEQ ID NO:1. Fragments can be at least 10%, at least 15%, at least 20%, at least 25%, at least 30%, at least 35%, at least 40%, at least 45%, at least 50%, at least 55% at least 60%, at least 65%, at least 70%, at least 75%, at least 80%, at least 85%, at least 90%, at least 95%, at least 96%, at least 97%, at least 98% or at least 99% of SEQ ID NO:1. Fragments can be at least 95%, at least 96%, at least 97%, at least 98%, or at least 99% homologous to fragments of SEQ ID NO:1. Fragments can be at least 80%, at least 85%, at least 90% at least 91%, at least 92%, at least 93%, at least 94%, at least 95%, at least 96%, at least 97%, at least 98%, or at least 99% identical to fragments of SEQ ID NO:1. In some embodiments, fragments include sequences that encode a leader sequence, such as for example, an immunoglobulin leader, such as the IgE leader. In some embodiments, fragments are free of coding sequences that encode a leader sequence. In some embodiments, fragments are free of coding sequences that encode a leader sequence, such as for example, the IgE leader.

Furthermore, in one embodiment, the amino acid sequence of the consensus dTERT protein is SEQ ID NO:2. The amino acid sequence of the consensus dTERT protein linked to an IgE leader is SEQ ID NO:4. The amino acid sequence of the consensus dTERT protein linked to the IgE leader may be linked to HA tag.

Some embodiments relate to proteins that are homologous to SEQ ID NO:2. Some embodiments relate to immunogenic proteins that have 95% homology to the protein sequences as set forth in SEQ ID NO:2. Some embodiments relate to immunogenic proteins that have 96% homology to the protein sequences as set forth in SEQ ID NO:2. Some embodiments relate to immunogenic proteins that have 97% homology to the protein sequences as set forth in SEQ ID NO:2. Some embodiments relate to immunogenic proteins that have 98% homology to the protein sequences as set forth in SEQ ID NO:2. Some embodiments relate to immunogenic proteins that have 99% homology to the protein sequences as set forth in SEQ ID NO:2.

Some embodiments relate to proteins that are identical to SEQ ID NO:2. Some embodiments relate to immunogenic proteins that have an amino acid sequence that is 80% identical to the full-length amino acid sequences as set forth in SEQ ID NO:2. Some embodiments relate to immunogenic proteins that have an amino acid sequence that is 85% identical to the full-length amino acid sequences as set forth in SEQ ID NO:2. Some embodiments relate to immunogenic proteins that have an amino acid sequence that is 90% identical to the full-length amino acid sequences as set forth in SEQ ID NO:2. Some embodiments relate to immunogenic proteins that have an amino acid sequence that is 91% identical to the full-length amino acid sequences as set forth in SEQ ID NO:2. Some embodiments relate to immunogenic proteins that have an amino acid sequence that is 92% identical to the full-length amino acid sequences as set forth in SEQ ID NO:2. Some embodiments relate to immunogenic proteins that have an amino acid sequence that is 93% identical to the full-length amino acid sequences as set forth in SEQ ID NO:2. Some embodiments relate to immunogenic proteins that have an amino acid sequence that is 94% identical to the full-length amino acid sequences as set forth in SEQ ID NO:2. Some embodiments relate to immunogenic proteins that have an amino acid sequence that is 95% identical to the full-length amino acid sequences as set forth in SEQ ID NO:2. Some embodiments relate to immunogenic proteins that have an amino acid sequence that is 96% identical to the full-length amino acid sequences as set forth in SEQ ID NO:2. Some embodiments relate to immunogenic proteins that have an amino acid sequence that is 97% identical to the full-length amino acid sequences as set forth in SEQ ID NO:2. Some embodiments relate to immunogenic proteins that have an amino acid sequence that is 98% identical to the full-length amino acid sequences as set forth in SEQ ID NO:2. Some embodiments relate to immunogenic proteins that have an amino acid sequence that is 99% identical to the full-length amino acid sequences as set forth in SEQ ID NO:2.

In some embodiments, the protein is free of a leader sequence. In some embodiments, the protein is free of the IgE leader. Fragments of proteins can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% of a protein. Immunogenic fragments of SEQ ID NO:2 can be provided. Immunogenic fragments can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% of SEQ ID NO:2. In some embodiments, fragments include a leader sequence, such as for example, an immunoglobulin leader, such as the IgE leader. In some embodiments, fragments are free of a leader sequence. In some embodiments, fragments are free of a leader sequence, such as for example, the IgE leader.

Immunogenic fragments of proteins with amino acid sequences homologous to immunogenic fragments of SEQ ID NO:2 can be provided. Such immunogenic fragments can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% of proteins that are 95% or greater homologous to SEQ ID NO:2. Some embodiments relate to immunogenic fragments that have 96% homology to the immunogenic fragments of protein sequences herein. Some embodiments relate to immunogenic fragments that have 97% homology to the immunogenic fragments of protein sequences herein. Some embodiments relate to immunogenic fragments that have 98% homology to the immunogenic fragments of protein sequences herein. Some embodiments relate to immunogenic fragments that have 99% homology to the immunogenic fragments of protein sequences herein. In some embodiments, fragments include a leader sequence, such as for example, an immunoglobulin leader, such as the IgE leader. In some embodiments, fragments are free of a leader sequence. In some embodiments, fragments are free of a leader sequence, such as for example, the IgE leader.

Immunogenic fragments of proteins with amino acid sequences identical to immunogenic fragments of SEQ ID NO:2 can be provided. Such immunogenic fragments can comprise at least 10%, at least 15%, at least 20%, at least 25%, at least 30%, at least 35%, at least 40%, at least 45%, at least 50%, at least 55% at least 60%, at least 65%, at least 70%, at least 75%, at least 80%, at least 85%, at least 90%, at least 95%, at least 96%, at least 97%, at least 98% or at least 99% of proteins that are 80%, 85%, 90%, 91%, 92%, 93%, 94%, 95%, 96%, 97%, 98%, or 99% identical to the amino acid sequences set forth in SEQ ID NO:2. In some embodiments, fragments include a leader sequence, such as for example, an immunoglobulin leader, such as the IgE leader. In some embodiments, fragments are free of a leader sequence. In some embodiments, fragments are free of a leader sequence, such as for example, the IgE leader.

As referred to herein with regard to linking a signal peptide or leader sequence to the N terminus of a protein, the signal peptide/leader sequence replaces the N terminal methionine of a protein which is encoded by the start codon of the nucleic acid sequence that encodes the protein without a signal peptide coding sequence.

Fragments of SEQ ID NO:1 may comprise 30 or more nucleotides, including preferably sequences that encode an immunodominant epitope. In some embodiments, fragments of SEQ ID NO:1 may comprise 45 or more nucleotides, including preferably sequences that encode an immunodominant epitope. In some embodiments, fragments of SEQ ID NO:1 may comprise 60 or more nucleotides, including preferably sequences that encode an immunodominant epitope. In some embodiments, fragments of SEQ ID NO:1 may comprise 75 or more nucleotides, including preferably sequences that encode an immunodominant epitope. In some embodiments, fragments of SEQ ID NO:1 may comprise 90 or more nucleotides, including preferably sequences that encode an immunodominant epitope. In some embodiments, fragments of SEQ ID NO:1 may comprise 120 or more nucleotides, including preferably sequences that encode an immunodominant epitope. In some embodiments, fragments of SEQ ID NO:1 may comprise 150 or more nucleotides, including preferably sequences that encode an immunodominant epitope. In some embodiments, fragments of SEQ ID NO:1 may comprise 180 or more nucleotides, including preferably sequences that encode an immunodominant epitope. In some embodiments, fragments of SEQ ID NO:1 may comprise 210 or more nucleotides, including preferably sequences that encode an immunodominant epitope. In some embodiments, fragments of SEQ ID NO:1 may comprise 240 or more nucleotides, including preferably sequences that encode an immunodominant epitope. In some embodiments, fragments of SEQ ID NO:1 may comprise 270 or more nucleotides, including preferably sequences that encode an immunodominant epitope. In some embodiments, fragments of SEQ ID NO:1 may comprise 300 or more nucleotides, including preferably sequences that encode an immunodominant epitope. In some embodiments, fragments of SEQ ID NO:1 may comprise 360 or more nucleotides, including preferably sequences that encode an immunodominant epitope. In some embodiments, fragments of SEQ ID NO:1 may comprise 420 or more nucleotides, including preferably sequences that encode an immunodominant epitope. In some embodiments, fragments of SEQ ID NO:1 may comprise 480 or more nucleotides, including preferably sequences that encode an immunodominant epitope. In some embodiments, fragments of SEQ ID NO:1 may comprise 540 or more nucleotides, including preferably sequences that encode an immunodominant epitope. In some embodiments, fragments of SEQ ID NO:1 may comprise 600 or more nucleotides, including preferably sequences that encode an immunodominant epitope. In some embodiments, fragments of SEQ ID NO:1 may comprise 300 or more nucleotides, including preferably sequences that encode an immunodominant epitope. In some embodiments, fragments of SEQ ID NO:1 may comprise 660 or more nucleotides, including preferably sequences that encode an immunodominant epitope. In some embodiments, fragments of SEQ ID NO:1 may comprise 720 or more nucleotides, including preferably sequences that encode an immunodominant epitope. In some embodiments, fragments of SEQ ID NO:1 may comprise 780 or more nucleotides, including preferably sequences that encode an immunodominant epitope. In some embodiments, fragments of SEQ ID NO:1 may comprise 840 or more nucleotides, including preferably sequences that encode an immunodominant epitope. In some embodiments, fragments of SEQ ID NO:1 may comprise 900 or more nucleotides, including preferably sequences that encode an immunodominant epitope. In some embodiments, fragments of SEQ ID NO:1 may comprise 960 or more nucleotides, including preferably sequences that encode an immunodominant epitope. In some embodiments, fragments of SEQ ID NO:1 may comprise 1020 or more nucleotides, including preferably sequences that encode an immunodominant epitope. In some embodiments, fragments of SEQ ID NO:1 may comprise 1080 or more nucleotides, including preferably sequences that encode an immunodominant epitope. In some embodiments, fragments of SEQ ID NO:1 may comprise 1140 or more nucleotides, including preferably sequences that encode an immunodominant epitope. In some embodiments, fragments of SEQ ID NO:1 may comprise 1200 or more nucleotides, including preferably sequences that encode an immunodominant epitope. In some embodiments, fragments of SEQ ID NO:1 may comprise 1260 or more nucleotides, including preferably sequences that encode an immunodominant epitope. In some embodiments, fragments of SEQ ID NO:1 may comprise 1320 or more nucleotides, including preferably sequences that encode an immunodominant epitope. In some embodiments, fragments of SEQ ID NO:1 may comprise 1380 or more nucleotides, including preferably sequences that encode an immunodominant epitope. In some embodiments, fragments of SEQ ID NO:1 may comprise 1440 or more nucleotides, including preferably sequences that encode an immunodominant epitope. In some embodiments, fragments of SEQ ID NO:1 may comprise 1500 or more nucleotides, including preferably sequences that encode an immunodominant epitope. In some embodiments, fragments of SEQ ID NO:1 may comprise 1560 or more nucleotides, including preferably sequences that encode an immunodominant epitope. In some embodiments, fragments of SEQ ID NO:1 may comprise 1620 or more nucleotides, including preferably sequences that encode an immunodominant epitope. In some embodiments, fragments of SEQ ID NO:1 may comprise 1680 or more nucleotides, including preferably sequences that encode an immunodominant epitope. In some embodiments, fragments of SEQ ID NO:1 may comprise 1740 or more nucleotides, including preferably sequences that encode an immunodominant epitope. In some embodiments, fragments of SEQ ID NO:1 may comprise 1800 or more nucleotides, including preferably sequences that encode an immunodominant epitope. In some embodiments, fragments of SEQ ID NO:1 may comprise 1860 or more nucleotides, including preferably sequences that encode an immunodominant epitope. In some embodiments, fragments of SEQ ID NO:1 may comprise 1920 or more nucleotides, including preferably sequences that encode an immunodominant epitope. In some embodiments, fragments of SEQ ID NO:1 may comprise 1980 or more nucleotides, including preferably sequences that encode an immunodominant epitope. In some embodiments, fragments of SEQ ID NO:1 may comprise 2040 or more nucleotides, including preferably sequences that encode an immunodominant epitope. In some embodiments, fragments of SEQ ID NO:1 may comprise 2100 or more nucleotides, including preferably sequences that encode an immunodominant epitope. In some embodiments, fragments of SEQ ID NO:1 may comprise 2160 or more nucleotides, including preferably sequences that encode an immunodominant epitope. In some embodiments, fragments of SEQ ID NO:1 may comprise 2220 or more nucleotides, including preferably sequences that encode an immunodominant epitope. In some embodiments, fragments of SEQ ID NO:1 may comprise 2280 or more nucleotides, including preferably sequences that encode an immunodominant epitope. In some embodiments, fragments of SEQ ID NO:1 may comprise 2340 or more nucleotides, including preferably sequences that encode an immunodominant epitope. In some embodiments, fragments of SEQ ID NO:1 may comprise 2400 or more nucleotides, including preferably sequences that encode an immunodominant epitope. In some embodiments, fragments of SEQ ID NO:1 may comprise 2460 or more nucleotides, including preferably sequences that encode an immunodominant epitope. In some embodiments, fragments of SEQ ID NO:1 may comprise 2520 or more nucleotides, including preferably sequences that encode an immunodominant epitope. In some embodiments, fragments of SEQ ID NO:1 may comprise 2580 or more nucleotides, including preferably sequences that encode an immunodominant epitope. In some embodiments, fragments of SEQ ID NO:1 may comprise 2640 or more nucleotides, including preferably sequences that encode an immunodominant epitope. In some embodiments, fragments of SEQ ID NO:1 may comprise 2700 or more nucleotides, including preferably sequences that encode an immunodominant epitope. In some embodiments, fragments of SEQ ID NO:1 may comprise 2760 or more nucleotides, including preferably sequences that encode an immunodominant epitope. In some embodiments, fragments of SEQ ID NO:1 may comprise 2820 or more nucleotides, including preferably sequences that encode an immunodominant epitope. In some embodiments, fragments of SEQ ID NO:1 may comprise 2880 or more nucleotides, including preferably sequences that encode an immunodominant epitope. In some embodiments, fragments of SEQ ID NO:1 may comprise 2940 or more nucleotides, including preferably sequences that encode an immunodominant epitope. In some embodiments, fragments of SEQ ID NO:1 may comprise 3000 or more nucleotides, including preferably sequences that encode an immunodominant epitope. In some embodiments, fragments of SEQ ID NO:1 may comprise 3060 or more nucleotides, including preferably sequences that encode an immunodominant epitope. In some embodiments, fragments of SEQ ID NO:1 may comprise 3120 or more nucleotides, including preferably sequences that encode an immunodominant epitope. In some embodiments, fragments of SEQ ID NO:1 may comprise 3180 or more nucleotides, including preferably sequences that encode an immunodominant epitope. In some embodiments, fragments of SEQ ID NO:1 may comprise 3240 or more nucleotides, including preferably sequences that encode an immunodominant epitope. In some embodiments, fragments of SEQ ID NO:1 may comprise 3300 or more nucleotides, including preferably sequences that encode an immunodominant epitope. In some embodiments, fragments of SEQ ID NO:1 may comprise 3360 or more nucleotides, including preferably sequences that encode an immunodominant epitope. In some embodiments, fragments of SEQ ID NO:1 may comprise coding sequences for the IgE leader sequence. In some embodiments, fragments of SEQ ID NO:1 do not comprise coding sequences for the IgE leader sequence.

Fragments may comprise fewer than 60 nucleotides, in some embodiments fewer than 75 nucleotides, in some embodiments fewer than 90 nucleotides, in some embodiments fewer than 120 nucleotides, in some embodiments fewer than 150 nucleotides, in some embodiments fewer than 180 nucleotides, in some embodiments fewer than 210 nucleotides, in some embodiments fewer than 240 nucleotides, in some embodiments fewer than 270 nucleotides, in some embodiments fewer than 300 nucleotides, in some embodiments fewer than 360 nucleotides, in some embodiments fewer than 420 nucleotides, in some embodiments fewer than 480 nucleotides, in some embodiments fewer than 540 nucleotides, in some embodiments fewer than 600 nucleotides, in some embodiments fewer than 660 nucleotides, in some embodiments fewer than 720 nucleotides, in some embodiments fewer than 780 nucleotides, in some embodiments fewer than 840 nucleotides, in some embodiments fewer than 900 nucleotides, in some embodiments fewer than 960 nucleotides, in some embodiments fewer than 1020 nucleotides, in some embodiments fewer than 1080 nucleotides, in some embodiments fewer than 1140 nucleotides, in some embodiments fewer than 1200 nucleotides, in some embodiments fewer than 1260 nucleotides, in some embodiments fewer than 1320 nucleotides, in some embodiments fewer than 1380 nucleotides, in some embodiments fewer than 1440 nucleotides, in some embodiments fewer than 1500 nucleotides, in some embodiments fewer than 1560 nucleotides, in some embodiments fewer than 1620 nucleotides, in some embodiments fewer than 1680 nucleotides, in some embodiments fewer than 1740 nucleotides, in some embodiments fewer than 1800 nucleotides, in some embodiments fewer than 1860 nucleotides, in some embodiments fewer than 1920 nucleotides, in some embodiments fewer than 1980 nucleotides, in some embodiments fewer than 2040 nucleotides, in some embodiments fewer than 2100 nucleotides, in some embodiments fewer than 2160 nucleotides, in some embodiments fewer than 2220 nucleotides, in some embodiments fewer than 2280 nucleotides, in some embodiments fewer than 2340 nucleotides, in some embodiments fewer than 2400 nucleotides, in some embodiments fewer than 2460 nucleotides, in some embodiments fewer than 2520 nucleotides, in some embodiments fewer than 2580 nucleotides, in some embodiments fewer than 2640 nucleotides, in some embodiments fewer than 2700 nucleotides, in some embodiments fewer than 2760 nucleotides, in some embodiments fewer than 2820 nucleotides, in some embodiments fewer than 2860 nucleotides, in some embodiments fewer than 2940 nucleotides, in some embodiments fewer than 3000 nucleotides, in some embodiments fewer than 3060 nucleotides, in some embodiments fewer than 3120 nucleotides, in some embodiments fewer than 3180 nucleotides, in some embodiments fewer than 3240 nucleotides, in some embodiments fewer than 3300 nucleotides, in some embodiments fewer than 3360 nucleotides.

Fragments of SEQ ID NO:2 may comprise 15 or more amino acids, including preferably sequences that encode an immunodominant epitope. In some embodiments, fragments of SEQ ID NO:2 may comprise 18 or more amino acids, including preferably sequences that encode an immunodominant epitope. In some embodiments, fragments of SEQ ID NO:2 may comprise 21 or more amino acids, including preferably sequences that encode an immunodominant epitope. In some embodiments, fragments of SEQ ID NO:2 may comprise 24 or more amino acids, including preferably sequences that encode an immunodominant epitope. In some embodiments, fragments of SEQ ID NO:2 may comprise 30 or more amino acids, including preferably sequences that encode an immunodominant epitope. In some embodiments, fragments of SEQ ID NO:2 may comprise 36 or more amino acids, including preferably sequences that encode an immunodominant epitope. In some embodiments, fragments of SEQ ID NO:2 may comprise 42 or more amino acids, including preferably sequences that encode an immunodominant epitope. In some embodiments, fragments of SEQ ID NO:2 may comprise 48 or more amino acids, including preferably sequences that encode an immunodominant epitope. In some embodiments, fragments of SEQ ID NO:2 may comprise 54 or more amino acids, including preferably sequences that encode an immunodominant epitope. In some embodiments, fragments of SEQ ID NO:2 may comprise 60 or more amino acids, including preferably sequences that encode an immunodominant epitope. In some embodiments, fragments of SEQ ID NO:2 may comprise 66 or more amino acids, including preferably sequences that encode an immunodominant epitope. In some embodiments, fragments of SEQ ID NO:2 may comprise 72 or more amino acids, including preferably sequences that encode an immunodominant epitope. In some embodiments, fragments of SEQ ID NO:2 may comprise 90 or more amino acids, including preferably sequences that encode an immunodominant epitope. In some embodiments, fragments of SEQ ID NO:2 may comprise 120 or more amino acids, including preferably sequences that encode an immunodominant epitope. In some embodiments, fragments of SEQ ID NO:2 may comprise 150 or more amino acids, including preferably sequences that encode an immunodominant epitope. In some embodiments, fragments of SEQ ID NO:2 may comprise 180 or more amino acids, including preferably sequences that encode an immunodominant epitope. In some embodiments, fragments of SEQ ID NO:2 may comprise 210 or more amino acids, including preferably sequences that encode an immunodominant epitope. In some embodiments, fragments of SEQ ID NO:2 may comprise 240 or more amino acids, including preferably sequences that encode an immunodominant epitope. In some embodiments, fragments of SEQ ID NO:2 may comprise 270 or more amino acids, including preferably sequences that encode an immunodominant epitope. In some embodiments, fragments of SEQ ID NO:2 may comprise 300 or more amino acids, including preferably sequences that encode an immunodominant epitope. In some embodiments, fragments of SEQ ID NO:2 may comprise 330 or more amino acids, including preferably sequences that encode an immunodominant epitope. In some embodiments, fragments of SEQ ID NO:2 may comprise 360 or more amino acids, including preferably sequences that encode an immunodominant epitope. In some embodiments, fragments of SEQ ID NO:2 may comprise 390 or more amino acids, including preferably sequences that encode an immunodominant epitope. In some embodiments, fragments of SEQ ID NO:2 may comprise 420 or more amino acids, including preferably sequences that encode an immunodominant epitope. In some embodiments, fragments of SEQ ID NO:2 may comprise 450 or more amino acids, including preferably sequences that encode an immunodominant epitope. In some embodiments, fragments of SEQ ID NO:2 may comprise 480 or more amino acids, including preferably sequences that encode an immunodominant epitope. In some embodiments, fragments of SEQ ID NO:2 may comprise 510 or more amino acids, including preferably sequences that encode an immunodominant epitope. In some embodiments, fragments of SEQ ID NO:2 may comprise 540 or more amino acids, including preferably sequences that encode an immunodominant epitope. In some embodiments, fragments of SEQ ID NO:2 may comprise 570 or more amino acids, including preferably sequences that encode an immunodominant epitope. In some embodiments, fragments of SEQ ID NO:2 may comprise 600 or more amino acids, including preferably sequences that encode an immunodominant epitope. In some embodiments, fragments of SEQ ID NO:2 may comprise 630 or more amino acids, including preferably sequences that encode an immunodominant epitope. In some embodiments, fragments of SEQ ID NO:2 may comprise 660 or more amino acids, including preferably sequences that encode an immunodominant epitope. In some embodiments, fragments of SEQ ID NO:2 may comprise 690 or more amino acids, including preferably sequences that encode an immunodominant epitope. In some embodiments, fragments of SEQ ID NO:2 may comprise 720 or more amino acids, including preferably sequences that encode an immunodominant epitope. In some embodiments, fragments of SEQ ID NO:2 may comprise 750 or more amino acids, including preferably sequences that encode an immunodominant epitope. In some embodiments, fragments of SEQ ID NO:2 may comprise 780 or more amino acids, including preferably sequences that encode an immunodominant epitope. In some embodiments, fragments of SEQ ID NO:2 may comprise 810 or more amino acids, including preferably sequences that encode an immunodominant epitope. In some embodiments, fragments of SEQ ID NO:2 may comprise 840 or more amino acids, including preferably sequences that encode an immunodominant epitope. In some embodiments, fragments of SEQ ID NO:2 may comprise 870 or more amino acids, including preferably sequences that encode an immunodominant epitope. In some embodiments, fragments of SEQ ID NO:2 may comprise 900 or more amino acids, including preferably sequences that encode an immunodominant epitope. In some embodiments, fragments of SEQ ID NO:2 may comprise 930 or more amino acids, including preferably sequences that encode an immunodominant epitope. In some embodiments, fragments of SEQ ID NO:2 may comprise 960 or more amino acids, including preferably sequences that encode an immunodominant epitope. In some embodiments, fragments of SEQ ID NO:2 may comprise 990 or more amino acids, including preferably sequences that encode an immunodominant epitope. In some embodiments, fragments of SEQ ID NO:2 may comprise 1020 or more amino acids, including preferably sequences that encode an immunodominant epitope. In some embodiments, fragments of SEQ ID NO:2 may comprise 1050 or more amino acids, including preferably sequences that encode an immunodominant epitope. In some embodiments, fragments of SEQ ID NO:2 may comprise 1080 or more amino acids, including preferably sequences that encode an immunodominant epitope. In some embodiments, fragments of SEQ ID NO:2 may comprise 1110 or more amino acids, including preferably sequences that encode an immunodominant epitope. In some embodiments, fragments of SEQ ID NO:2 may comprise 1140 or more amino acids, including preferably sequences that encode an immunodominant epitope. In some embodiments, fragments of SEQ ID NO:2 may comprise 1170 or more amino acids, including preferably sequences that encode an immunodominant epitope. In some embodiments, fragments of SEQ ID NO:2 may comprise 1200 or more amino acids, including preferably sequences that encode an immunodominant epitope. In some embodiments, fragments of SEQ ID NO:2 may comprise 1230 or more amino acids, including preferably sequences that encode an immunodominant epitope. In some embodiments, fragments of SEQ ID NO:2 may comprise 1260 or more amino acids, including preferably sequences that encode an immunodominant epitope. In some embodiments, fragments of SEQ ID NO:2 may comprise 1290 or more amino acids, including preferably sequences that encode an immunodominant epitope. In some embodiments, fragments of SEQ ID NO:2 may comprise 1320 or more amino acids, including preferably sequences that encode an immunodominant epitope. In some embodiments, fragments of SEQ ID NO:2 may comprise 1350 or more amino acids, including preferably sequences that encode an immunodominant epitope. In some embodiments, fragments of SEQ ID NO:2 may comprise 1380 or more amino acids, including preferably sequences that encode an immunodominant epitope. In some embodiments, fragments of SEQ ID NO:2 may comprise 1410 or more amino acids, including preferably sequences that encode an immunodominant epitope. In some embodiments, fragments of SEQ ID NO:2 may comprise 1440 or more amino acids, including preferably sequences that encode an immunodominant epitope. In some embodiments, fragments of SEQ ID NO:2 may comprise 1470 or more amino acids, including preferably sequences that encode an immunodominant epitope. In some embodiments, fragments of SEQ ID NO:2 may comprise 1500 or more amino acids, including preferably sequences that encode an immunodominant epitope. In some embodiments, fragments of SEQ ID NO:2 may comprise coding sequences for the IgE leader sequences. In some embodiments, fragments of SEQ ID NO:2 do not comprise coding sequences for the IgE leader sequences.

Fragments may comprise fewer than 24 amino acids, in some embodiments fewer than 30 amino acids, in some embodiments fewer than 36 amino acids, in some embodiments fewer than 42 amino acids, in some embodiments fewer than 48 amino acids, in some embodiments fewer than 54 amino acids, in some embodiments fewer than 60 amino acids, in some embodiments fewer than 72 amino acids, in some embodiments fewer than 90 amino acids, in some embodiments fewer than 120 amino acids, in some embodiments fewer than 150 amino acids, in some embodiments fewer than 180 amino acids, in some embodiments fewer than 210 amino acids in some embodiments fewer than 240 amino acids, in some embodiments fewer than 260 amino acids, in some embodiments fewer than 290 amino acids, in some embodiments fewer than 320 amino acids, in some embodiments fewer than 350 amino acids, in some embodiments fewer than 380 amino acids, in some embodiments fewer than 410 amino acids in some embodiments fewer than 440 amino acids, in some embodiments fewer than 470 amino acids in some embodiments fewer than 500 amino acids, in some embodiments fewer than 530 amino acids in some embodiments fewer than 560 amino acids, in some embodiments fewer than 590 amino acids, in some embodiments fewer than 620 amino acids, in some embodiments fewer than 650 amino acids, in some embodiments fewer than 680 amino acids, in some embodiments fewer than 710 amino acids, in some embodiments fewer than 740 amino acids, in some embodiments fewer than 770 amino acids, in some embodiments fewer than 800 amino acids, in some embodiments fewer than 830 amino acids, in some embodiments fewer than 860 amino acids, in some embodiments fewer than 890 amino acids, in some embodiments fewer than 920 amino acids, in some embodiments fewer than 950 amino acids, in some embodiments fewer than 980 amino acids, in some embodiments fewer than 1010 amino acids, in some embodiments fewer than 1040 amino acids, in some embodiments fewer than 1070 amino acids, in some embodiments fewer than 1200 amino acids, in some embodiments fewer than 1230 amino acids, in some embodiments fewer than 1260 amino acids, in some embodiments fewer than 1290 amino acids, in some embodiments fewer than 1320 amino acids, in some embodiments fewer than 1350 amino acids, in some embodiments fewer than 1380 amino acids, in some embodiments fewer than 1410 amino acids, in some embodiments fewer than 1440 amino acids, in some embodiments fewer than 1470 amino acids, and in some embodiments fewer than 1500 amino acids.

Combination with Tumor Antigen

The vaccine can comprise a synthetic consensus dTERT antigen alone or in combination with one or more tumor antigens. In the context of the present invention, “tumor antigen”, “cancer antigen” or “hyperproliferative disorder antigen” or “antigen associated with a hyperproliferative disorder,” refers to antigens that are common to specific hyperproliferative disorders such as cancer. The antigens discussed herein are merely included by way of example. The list is not intended to be exclusive and further examples will be readily apparent to those of skill in the art.

Tumor antigens are proteins that are produced by tumor cells that elicit an immune response, particularly T-cell mediated immune responses. The selection of the antigen binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, tyrosinase (TYR), TYRP1, TYRP2, RAGE-1, MN-CA IX, RU1, RU2 (AS), intestinal carboxyl esterase, mut hsp70-2, M-CSF, prostase, prostate-specific antigen (PSA), PAP, NY-ESO-1, NY-ESO-2, LAGE-1a, p53, prostein, PSMA, GHRH, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TYRP1, TYRP2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; and unique tumor antigens resulting from chromosomal translocations; such as BCR-ABL, E2A-PRL, H4-RET, IGH-IGK, and MYL-RAR. Other large, protein-based antigens include TSP-180, MAGE-4, MAGE-5, MAGE-6, RAGE, NY-ESO-1, NY-ESO-2, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

The dTERT antigen or fragment of variant thereof of the invention can be associated or combined with one or more additional tumor antigen or fragment or variant thereof. By methodology of generating antigens that represent such markers in a way to break tolerance to self, a cancer vaccine can be generated. Such cancer vaccines can optionally include one or more antibodies targeting one or more additional immune checkpoint proteins to enhance the immune response.

Immune suppression can be facilitated by myeloid derived suppressor cells (MDSCs), which are a mixed population of immature macrophages, granulocytes, dendritic cells, and myeloid cells. The myeloid cells can be a heterogenous population of myeloid progenitor cells and immature myeloid cells (IMCs). Markers of MDSCs can include expression of Gr-1 and CD11b (i.e., Gr-1⁺ and CD11b⁺ cells).

Circulation of MDSCs can increase due to chronic infection and expansion of MDSC populations can be associated with autoimmunity and inflammation. Particularly, MDSC expansion (or presence in the tumor or cancerous tissue) can facilitate tumor growth and escape from immune detection and/or regulation, and thus, MDSCs can affect immune responses to anticancer vaccines.

MDSCs can be regulated by Regulator of G-protein signaling 2 (Rgs2) and Rgs2 can be highly expressed in MDSCs derived from tumors. Rgs2 can also be widely expressed in a variety of cells, for example, myeloid cells. MDSCs derived from tumor bearing mice can function differently from MDSCs derived from non-tumor bearing mice. One such difference can be the up-regulation of the production of the chemokine MCP-1, which is secreted by MDSCs. MCP-1 can promote cell migration by signaling through CCR2, a G-protein coupled receptor (GPCR) found on monocytes, endothelial cells, and T cells. Accordingly, MCP-1 can cause migration of endothelial cells, thereby promoting vascularization. Blocking MCP-1 via neutralizing antibodies can inhibit angiogenesis, and thus, can lead to decreased tumor metastases and increased survival. As such, MCP-1 can be considered an angiogenic factor. Besides secreting MCP-1, MDSCs can secrete growth factors, thereby further contributing to tumor growth.

The following are some exemplary tumor antigens:

(1) Tyrosinase (Tyr)

The vaccine of the present invention can comprise the cancer antigen tyrosinase (Tyr), a fragment thereof, or a variant thereof. Tyrosinase is a copper-containing enzyme having tyrosine hydroxylase and dopa oxidase catalytic activities that can be found in microorganisms and plant and animal tissues. Specifically, tyrosinase catalyzes the production of melanin and other pigments by the oxidation of phenols such as tyrosine. Mutations in the TYR gene result in oculocutaneous albinism in mammals and non-pathological polymorphisms in the TYR gene contribute to variation in skin pigmentation.

Additionally, in cancer or tumors such as melanoma, tyrosinase can become unregulated, resulting in increased melanin synthesis. Accordingly, tyrosinase can be a cancer antigen associated with melanoma. In subjects suffering from melanoma, tyrosinase can be a target of cytotoxic T cell recognition. In some instances, however, the immune response to the cancer or tumor (including melanoma) can be suppressed, leading to a microenvironment that supports tumor formation and/or growth and thus, disease progression.

In one embodiment, the tyrosinase antigen is a canine tyrosinase antigen. In one embodiment, the tyrosinase antigen is a consensus tyrosinase antigen derived from multiple canine tyrosinase antigen sequences. In one embodiment, a tyrosinase antigen is operably linked to a signal peptide.

In one embodiment, a tyrosinase antigen can be administered in vectors described herein, and combined with the dTERT antigen and optionally one or more antibodies targeting one or more additional immune checkpoint proteins in various vaccination schedules.

The Tyr antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

As demonstrated herein, the Tyr antigen induces antigen-specific T-cell and high titer antibody responses against cancerous or tumor cells (e.g., melanoma cells). Specifically, the Tyr antigen is an important target for immune mediated clearance by inducing (1) humoral immunity via B cell responses to generate antibodies that block monocyte chemoattractant protein-1 (MCP-1) production, thereby retarding myeloid derived suppressor cells (MDSCs) and suppressing tumor growth; (2) increase cytotoxic T lymphocyte such as CD8⁺ (CTL) to attack and kill tumor cells; (3) increase T helper cell responses; and (4) increase inflammatory responses via IFN-γ and TNF-α or all of the aforementioned. As such, a protective immune response is provided against tumor formation and tumor growth by vaccines comprising the Tyr antigen (e.g., the consensus Tyr antigen, which is described below in more detail) because these vaccines prevent immune suppression by decreasing the population of MDSCs found within the cancerous or tumor tissue and block vascularization of the cancerous or tumor tissue by reducing production or secretion of MCP-1. Accordingly, any user can design a vaccine of the present invention to include a Tyr antigen to provide broad immunity against tumor formation, metastasis of tumors, and tumor growth.

The Tyr antigen can comprise protein epitopes that make them particularly effective as immunogens against which anti-Tyr immune responses can be induced. The Tyr antigen can comprise the full-length translation product, a variant thereof, a fragment thereof or a combination thereof. The Tyr antigen can comprise a consensus protein.

The nucleic acid sequence encoding the Tyr antigen can be optimized with regards to codon usage and corresponding RNA transcripts. The nucleic acid encoding the Tyr antigen can be codon and RNA optimized for expression in canines. In some embodiments, the nucleic acid sequence encoding the Tyr antigen can include a Kozak sequence to increase the efficiency of translation. The nucleic acid encoding the consensus Tyr antigen can include multiple stop codons to increase the efficiency of translation termination.

The nucleic acid encoding the Tyr antigen can also encode an immunoglobulin E (IgE) leader sequence. The nucleic acid encoding the consensus Tyr antigen can further encode the IgE leader sequence such that the amino acid sequence of the IgE leader sequence is linked to the amino acid sequence of the Tyr antigen by a peptide bond. The nucleic acid encoding the Tyr antigen can also include a nucleotide sequence encoding the IgE leader sequence. In some embodiments, the nucleic acid encoding the Tyr antigen is free of or does not contain a nucleotide sequence encoding the IgE leader sequence.

(2) Tyrosinase-Related Protein 1 (TYRP1)

The vaccine of the present invention can comprise the cancer antigen tyrosinase-related Protein 1 (TYRP1), a fragment thereof, or a variant thereof. TYRP1, encoded by the TYRP1 gene, is a 75 kDa transmembrane glycoprotein and is expressed in both normal and malignant melanocytes and melanoma cells. Like tyrosinase, TYRP1 contains a motif termed M-box that can bind to the microphtalmia transcription factor (MITF), which plays a central role within the melanocyte in activating pigmentation, cell proliferation and differentiation. TYRP1 may help to stabilize tyrosinase and can form a heterodimer, which may prevent the premature death of melanocytes by attenuating tyrosinase-mediated cytotoxicity.

As described above for tyrosinase, tyrosinase-related protein 1 (TYRP-1) can also be involved in the synthesis of melanin and pigmentary machinery of the melanocyte, and can be recognized by the immune system in subjects suffering from melanoma. Accordingly, TYRP-1 can be an antigen associated with melanoma.

In one embodiment, the TYRP-1 antigen is a canine TYRP-1 antigen. In one embodiment, the TYRP-1 antigen is a consensus TYRP-1 antigen derived from multiple canine TYRP-1 antigen sequences. In one embodiment, a TYRP-1 antigen is operably linked to a signal peptide.

In one embodiment, a TYRP-1 antigen can be administered in vectors described herein, and combined with the dTERT antigen and optionally one or more antibodies targeting one or more additional immune checkpoint proteins in various vaccination schedules.

The TYRP-1 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up-regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The TYRP-1 antigen can comprise protein epitopes that make them particularly effective as immunogens against which anti-TYRP-1 immune responses can be induced. The TYRP-1 antigen can comprise the full-length translation product, a variant thereof, a fragment thereof or a combination thereof. The TYRP-1 antigen can comprise a consensus protein.

The nucleic acid sequence encoding the consensus TYRP-1 antigen can be optimized with regards to codon usage and corresponding RNA transcripts. The nucleic acid encoding the consensus TYRP-1 antigen can be codon and RNA optimized for expression in canines. In some embodiments, the nucleic acid sequence encoding the consensus TYRP-1 antigen can include a Kozak sequence to increase the efficiency of translation. The nucleic acid encoding the consensus TYRP-1 antigen can include one or more stop codons to increase the efficiency of translation termination.

The nucleic acid encoding the consensus TYRP-1 antigen can also encode an immunoglobulin E (IgE) leader sequence. The nucleic acid encoding the consensus TYRP-1 antigen can further encode the IgE leader sequence such that the amino acid sequence of the IgE leader sequence is linked to the amino acid sequence of the consensus TYRP-1 antigen by a peptide bond. The nucleic acid encoding the consensus TYRP-1 antigen can also include a nucleotide sequence encoding the IgE leader sequence. In some embodiments, the nucleic acid encoding the consensus TYRP-1 antigen is free of or does not contain a nucleotide sequence encoding the IgE leader sequence.

As referred to herein with regard to linking a signal peptide or leader sequence to the N terminus of a protein, the signal peptide/leader sequence replaces the N terminal methionine of a protein which is encoded by the start codon of the nucleic acid sequence that encodes the protein without a signal peptide coding sequence.

(3) Tyrosinase-Related Protein 2 (TYRP2)

The vaccine of the present invention can comprise the cancer antigen tyrosinase-related Protein 2 (TYRP2; also known as dopachrome tautomerase (DCT)), a fragment thereof, or a variant thereof. TYRP2/DCT, encoded by the TYRP2/DCT gene, is a protein comprised of 519 amino acids and is expressed in both normal and malignant melanocytes and melanoma cells. TYRP2/DCT is a well-characterized melanocyte-specific enzyme that, in conjunction with tyrosinase and TYRP1, functions in the conversion of L-tyrosine to melanin in melanocytes. DCT specifically catalyzes the tautomerization of the melanin precursors L-dopachrome to 5,6-dihydroindole-2-carboxylic acid (DHICA), which is subsequently oxidized by TYRP1 (as discussed above) to form eumelanin. Studies have shown that TYRP2/DCT may be a mediator of drug resistance in melanoma cells, with specificity for DNA-damaging agents. Since TYRP2/DCT has frequently been reported to be highly expressed in melanomas, this melanocyte-specific enzyme plays an important role contributing to intrinsic resistance phenotype of melanomas to various anticancer DNA-damaging drugs.

As described above for tyrosinase, tyrosinase-related protein 2 (TYRP-2) can also be involved in the synthesis of melanin and recognized by the immune system in subjects suffering from melanoma. Additionally, TYRP-2 can mediate drug resistance in melanoma cells. Accordingly, TYRP-2 can be an antigen associated with melanoma.

In one embodiment, the TYRP-2 antigen is a canine TYRP-2 antigen. In one embodiment, the TYRP-2 antigen is a consensus TYRP-2 antigen derived from multiple canine TYRP-2 antigen sequences. In one embodiment, a TYRP-2 antigen is operably linked to a signal peptide.

In one embodiment, a TYRP-2 antigen can be administered in vectors described herein, and combined with the dTERT antigen and optionally one or more antibodies targeting one or more additional immune checkpoint proteins in various vaccination schedules.

The TRYP-2 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The TYRP2 antigen can comprise protein epitopes that make them particularly effective as immunogens against which anti-TYRP2 immune responses can be induced. The TYRP2 antigen can comprise the full-length translation product, a variant thereof, a fragment thereof or a combination thereof. The TYRP2 antigen can comprise a consensus protein.

The nucleic acid sequence encoding the consensus TYRP2 antigen can be optimized with regards to codon usage and corresponding RNA transcripts. The nucleic acid encoding the consensus TYRP2 antigen can be codon and RNA optimized for expression in canines. In some embodiments, the nucleic acid sequence encoding the consensus TYRP2 antigen can include a Kozak sequence to increase the efficiency of translation. The nucleic acid encoding the consensus TYRP2 antigen can include multiple stop codons to increase the efficiency of translation termination.

The nucleic acid encoding the consensus TYRP2 antigen can also encode an immunoglobulin E (IgE) leader sequence. The nucleic acid encoding the consensus TYRP2 antigen can further encode the IgE leader sequence such that the amino acid sequence of the IgE leader sequence is linked to the amino acid sequence of the consensus TYRP2 antigen by a peptide bond. The nucleic acid encoding the consensus TYRP2 antigen can also include a nucleotide sequence encoding the IgE leader sequence. In some embodiments, the nucleic acid encoding the consensus TYRP2 antigen is free of or does not contain a nucleotide sequence encoding the IgE leader sequence.

As referred to herein with regard to linking a signal peptide or leader sequence to the N terminus of a protein, the signal peptide/leader sequence replaces the N terminal methionine of a protein which is encoded by the start codon of the nucleic acid sequence that encodes the protein without a signal peptide coding sequence.

(4) Melanoma-Associated Antigen 4 (MAGEA4)

The vaccine of the present invention can comprise the cancer antigen Melanoma-associated Antigen 4 (MAGEA4), a fragment thereof, or a variant thereof. MAGE-A4, encoded by the MAGE-A4 gene, is a protein comprised of 317 amino acids and is expressed in male germ cells and tumor cells of various histological types such as gastrointestinal, esophageal and pulmonary carcinomas. MAGE-A4 binds the oncoprotein, Gankyrin. This MAGE-A4 specific binding is mediated by its C-terminus. Studies have shown that exogenous MAGE-A4 can partly inhibit the adhesion-independent growth of Gankyrin-overexpressing cells in vitro and suppress the formation of migrated tumors from these cells in nude mice. This inhibition is dependent upon binding between MAGE-A4 and Gankyrin, suggesting that interactions between Gankyrin and MAGE-A4 inhibit Gankyrin-mediated carcinogenesis. It is likely that MAGE expression in tumor tissue is not a cause, but a result of tumor genesis, and MAGE genes take part in the immune process by targeting early tumor cells for destruction.

Melanoma-associated antigen 4 protein (MAGEA4) can be involved in embryonic development and tumor transformation and/or progression. MAGEA4 is normally expressed in testes and placenta. MAGEA4, however, can be expressed in many different types of tumors, for example, melanoma, head and neck squamous cell carcinoma, lung carcinoma, and breast carcinoma. Accordingly, MAGEA4 can be antigen associated with a variety of tumors.

In one embodiment, the MAGEA4 antigen is a canine MAGEA4 antigen. In one embodiment, the MAGEA4 antigen is a consensus MAGEA4 antigen derived from multiple canine MAGEA4 antigen sequences. In one embodiment, a MAGEA4 antigen is operably linked to a signal peptide.

In one embodiment, a MAGEA4 antigen can be administered in vectors described herein, and combined with the dTERT antigen and optionally one or more antibodies targeting one or more additional immune checkpoint proteins in various vaccination schedules.

The MAGEA4 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The MAGEA4 antigen can comprise protein epitopes that make them particularly effective as immunogens against which anti-MAGEA4 immune responses can be induced. The MAGEA4 antigen can comprise the full-length translation product, a variant thereof, a fragment thereof or a combination thereof. The MAGEA4 antigen can comprise a consensus protein.

The nucleic acid sequence encoding the consensus MAGEA4 antigen can be optimized with regards to codon usage and corresponding RNA transcripts. The nucleic acid encoding the consensus MAGEA4 antigen can be codon and RNA optimized for expression in canines. In some embodiments, the nucleic acid sequence encoding the consensus MAGEA4 antigen can include a Kozak sequence to increase the efficiency of translation. The nucleic acid encoding the consensus MAGEA4 antigen can include one or more stop codons to increase the efficiency of translation termination.

The nucleic acid encoding the consensus MAGEA4 antigen can also encode an immunoglobulin E (IgE) leader sequence. The nucleic acid encoding the consensus Tyr antigen can further encode the IgE leader sequence such that the amino acid sequence of the IgE leader sequence is linked to the amino acid sequence of the consensus MAGEA4 antigen by a peptide bond. The nucleic acid encoding the consensus MAGEA4 antigen can also include a nucleotide sequence encoding the IgE leader sequence. In some embodiments, the nucleic acid encoding the consensus MAGEA4 antigen is free of or does not contain a nucleotide sequence encoding the IgE leader sequence.

(5) Growth Hormone Releasing Hormone (GHRH)

The vaccine of the present invention can comprise the cancer antigen growth hormone releasing hormone (GHRH; also known as growth-hormone-releasing factor (GRF or GHRF) or somatocrinin), a fragment thereof, or a variant thereof. GHRH is a 44 amino acid peptide hormone produced in the arcuate nucleus of the hypothalamus. GHRH is secreted by the hypothalamus and stimulates the release of growth hormone, a regulator of growth, metabolism, and body structure, from the pituitary gland. GHRH also stimulates the product of growth hormone. Antagonists of GHRH can inhibit the growth of a variety of cancers, for example, osteosarcomas, glioblastomas, prostate cancer, renal cancer, pancreatic cancer, colorectal cancer, and breast cancer. Accordingly, GHRH can be an antigen associated with a variety of tumors.

In one embodiment, the GHRH antigen is a canine GHRH antigen. In one embodiment, the GHRH antigen is a consensus GHRH antigen derived from multiple canine GHRH antigen sequences. In one embodiment, a GHRH antigen is operably linked to a signal peptide.

In one embodiment, a GHRH antigen can be administered in vectors described herein, and combined with the dTERT antigen and optionally one or more antibodies targeting one or more additional immune checkpoint proteins in various vaccination schedules.

The GHRH antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The GHRH antigen can comprise protein epitopes that make them particularly effective as immunogens against which anti-GHRH immune responses can be induced. The GHRH antigen can comprise the full-length translation product, a variant thereof, a fragment thereof or a combination thereof. The GHRH antigen can comprise a consensus protein.

The nucleic acid sequence encoding the consensus GHRH antigen can be optimized with regards to codon usage and corresponding RNA transcripts. The nucleic acid encoding the consensus GHRH antigen can be codon and RNA optimized for expression in canines. In some embodiments, the nucleic acid sequence encoding the consensus GHRH antigen can include a Kozak sequence to increase the efficiency of translation. The nucleic acid encoding the consensus GHRH antigen can include one or more stop codons to increase the efficiency of translation termination.

The nucleic acid encoding the consensus GHRH antigen can also encode an immunoglobulin E (IgE) leader sequence. The nucleic acid encoding the consensus GHRH antigen can further encode the IgE leader sequence such that the amino acid sequence of the IgE leader sequence is linked to the amino acid sequence of the consensus GHRH antigen by a peptide bond. The nucleic acid encoding the consensus GHRH antigen can also include a nucleotide sequence encoding the IgE leader sequence. In some embodiments, the nucleic acid encoding the consensus GHRH antigen is free of or does not contain a nucleotide sequence encoding the IgE leader sequence.

(6) MART-1/Melan-A

The vaccine of the present invention can comprise the cancer antigen MART-1 (also known as Melan-A), a fragment thereof, or a variant thereof. MART-1, encoded by MLANA gene, is a 118-amino acid protein containing a single transmembrane domain and is expressed in most melanoma cells. MART-1 forms a complex with a structural protein and affects its expression, stability, trafficking and processing which is required for melanosome structure and maturation. Accordingly, MART-1 is indispensable for regulating mammalian pigmentation. Defects in melanosome maturation have been linked to susceptibility to cancer. MART-1 may be expressed in numerous cancers, including, but not limited to, melanomas.

In one embodiment, the MART-1 antigen is a canine MART-1 antigen. In one embodiment, the MART-1 antigen is a consensus MART-1 antigen derived from multiple canine MART-1 antigen sequences. In one embodiment, a MART-1 antigen is operably linked to a signal peptide.

In one embodiment, a MART-1 antigen can be administered in vectors described herein, and combined with the dTERT antigen and optionally one or more antibodies targeting one or more additional immune checkpoint proteins in various vaccination schedules.

Melan-A, also known as melanoma antigen recognized by T cells (MART-1) is a melanocyte differentiation antigen and can be found in normal skin, retina, and melanocytes. Melan-A can be associated with the endoplasmic reticulum and melanosomes. Melan-A can be recognized by cytotoxic T cells as an antigen on melanoma cells, but can also be associated with other tumors having melanocytic origin or differentiation (i.e., cells have melansomes), for example, clear cell sarcoma and melanotic neurofibroma. Accordingly, Melan-A can be an antigen associated with a variety of tumors derived from cells having melanosomes.

In one embodiment, the MELAN-A antigen is a canine MELAN-A antigen. In one embodiment, the MELAN-A antigen is a consensus MELAN-A antigen derived from multiple canine MELAN-A antigen sequences. In one embodiment, a MELAN-A antigen is operably linked to a signal peptide.

In one embodiment, a MELAN-A antigen can be administered in vectors described herein, and combined with the dTERT antigen and optionally one or more antibodies targeting one or more additional immune checkpoint proteins in various vaccination schedules.

The Melan-A antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The Melan-A antigen can comprise protein epitopes that make them particularly effective as immunogens against which anti-Melan-A immune responses can be induced. The Melan-A antigen can comprise the full-length translation product, a variant thereof, a fragment thereof or a combination thereof. The Melan-A antigen can comprise a consensus protein.

The nucleic acid sequence encoding the consensus Melan-A antigen can be optimized with regards to codon usage and corresponding RNA transcripts. The nucleic acid encoding the consensus Melan-A antigen can be codon and RNA optimized for expression in canines. In some embodiments, the nucleic acid sequence encoding the consensus Melan-A antigen can include a Kozak sequence to increase the efficiency of translation. The nucleic acid encoding the consensus Melan-A antigen can include one or more stop codons to increase the efficiency of translation termination.

The nucleic acid encoding the consensus Melan-A antigen can also encode an immunoglobulin E (IgE) leader sequence. The nucleic acid encoding the consensus Melan-A antigen can further encode the IgE leader sequence such that the amino acid sequence of the IgE leader sequence is linked to the amino acid sequence of the consensus Melan-A antigen by a peptide bond. The nucleic acid encoding the consensus Melan-A antigen can also include a nucleotide sequence encoding the IgE leader sequence. In some embodiments, the nucleic acid encoding the consensus Melan-A antigen is free of or does not contain a nucleotide sequence encoding the IgE leader sequence.

(7) NY-ESO-1

The vaccine of the present invention can comprise the cancer antigen New York-esophageal cancer-1 (NY-ESO-1; also called CTAG1), a fragment thereof, or a variant thereof. NY-ESO-1, encoded by the CTAG1B gene, is a 180 amino-acid long protein, with a glycine-rich N-terminal region and an extremely hydrophobic C-terminal region. NY-ESO-1 has restricted expression in normal tissues but frequent occurrence in cancer. NY-ESO-1 may be expressed in numerous cancers including, but not limited to, bladder, colorectal, esophagus, gastric, hepatocarcinoma, head and neck, melanoma, non-small cell lung, ovarian, pancreatic, synovial carcinoma and prostate cancers.

Cancer-testis antigen (NY-ESO-1) can be expressed in the testis and ovary. NY-ESO-1 can be associated with a variety of cancers and can induce humoral immune responses. Subjects suffering from cancer or tumors can develop immunogenicity to NY-ESO-1. Accordingly, NY-ESO-1 can be an antigen associated with a variety of tumors.

In one embodiment, the NY-ESO-1 antigen is a canine NY-ESO-1 antigen. In one embodiment, the NY-ESO-1 antigen is a consensus NY-ESO-1 antigen derived from multiple canine NY-ESO-1 antigen sequences. In one embodiment, a NY-ESO-1 antigen is operably linked to a signal peptide.

In one embodiment, a NY-ESO-1 antigen can be administered in vectors described herein, and combined with the dTERT antigen and optionally one or more antibodies targeting one or more additional immune checkpoint proteins in various vaccination schedules.

The NY-ESO-1 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TGF-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The NY-ESO-1 antigen can comprise protein epitopes that make them particularly effective as immunogens against which anti-NY-ESO-1 immune responses can be induced. The NY-ESO-1 antigen can comprise the full-length translation product, a variant thereof, a fragment thereof or a combination thereof. The NY-ESO-1 antigen can comprise a consensus protein.

The nucleic acid sequence encoding the consensus NY-ESO-1 antigen can be optimized with regards to codon usage and corresponding RNA transcripts. The nucleic acid encoding the consensus NY-ESO-1 antigen can be codon and RNA optimized for expression in canines. In some embodiments, the nucleic acid sequence encoding the consensus NY-ESO-1 antigen can include a Kozak sequence to increase the efficiency of translation. The nucleic acid encoding the consensus NY-ESO-1 antigen can include one or more stop codons to increase the efficiency of translation termination.

The nucleic acid encoding the consensus NY-ESO-1 antigen can also encode an immunoglobulin E (IgE) leader sequence. The nucleic acid encoding the consensus NY-ESO-1 antigen can further encode the IgE leader sequence such that the amino acid sequence of the IgE leader sequence is linked to the amino acid sequence of the consensus NY-ESO-1 antigen by a peptide bond. The nucleic acid encoding the consensus NY-ESO-1 antigen can also include a nucleotide sequence encoding the IgE leader sequence. In some embodiments, the nucleic acid encoding the consensus NY-ESO-1 antigen is free of or does not contain a nucleotide sequence encoding the IgE leader sequence.

(8) NY-ESO-2

The vaccine of the present invention can comprise the cancer antigen New York-esophageal cancer-2 (NY-ESO-2; also known as cancer/testis antigen 2, ESO2, and LAGE1), a fragment thereof, or a variant thereof. NY-ESO-2 is an autoimmunogenic tumor antigen that belongs to the ESO/LAGE family of cancer-testis antigens. NY-ESO-2 can be expressed in a variety of cancers including melanoma, breast cancer, bladder cancer and prostate cancer and is normally expressed in testis tissue. Additionally, NY-ESO-2 can be observed in 25-50% of tumor samples of melanomas, non-small-cell lung carcinomas, bladder, prostate and head and neck cancers. The gene encoding NY-ESO-2 also contains an alternative open reading frame that encodes the protein named CAMEL, a tumor antigen that is recognized by melanoma-specific cytotoxic T-lymphocytes.

Similar to NY-ESO-1, NY-ESO-2 can be expressed in the testis and ovary. NY-ESO-2 can also be associated with a variety of cancers and immunogenic in subjects suffering from cancer or tumors. Accordingly, NY-ESO-2 can be an antigen associated with numerous tumors.

In one embodiment, the NY-ESO-2 antigen is a canine NY-ESO-2 antigen. In one embodiment, the NY-ESO-2 antigen is a consensus NY-ESO-2 antigen derived from multiple canine NY-ESO-2 antigen sequences. In one embodiment, a NY-ESO-2 antigen is operably linked to a signal peptide.

In one embodiment, a NY-ESO-2 antigen can be administered in vectors described herein, and combined with the dTERT antigen and optionally one or more antibodies targeting one or more additional immune checkpoint proteins in various vaccination schedules.

The NY-ESO-2 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The NY-ESO-2 antigen can comprise protein epitopes that make them particularly effective as immunogens against which anti-NY-ESO-2 immune responses can be induced. The NY-ESO-2 antigen can comprise the full-length translation product, a variant thereof, a fragment thereof or a combination thereof. The NY-ESO-2 antigen can comprise a consensus protein.

The nucleic acid sequence encoding the consensus NY-ESO-2 antigen can be optimized with regards to codon usage and corresponding RNA transcripts. The nucleic acid encoding the consensus NY-ESO-2 antigen can be codon and RNA optimized for expression in canines. In some embodiments, the nucleic acid sequence encoding the consensus NY-ESO-2 antigen can include a Kozak sequence to increase the efficiency of translation. The nucleic acid encoding the consensus NY-ESO-2 antigen can include one or more stop codons to increase the efficiency of translation termination.

The nucleic acid encoding the consensus NY-ESO-2 antigen can also encode an immunoglobulin E (IgE) leader sequence. The nucleic acid encoding the consensus NY-ESO-2 antigen can further encode the IgE leader sequence such that the amino acid sequence of the IgE leader sequence is linked to the amino acid sequence of the consensus NY-ESO-2 antigen by a peptide bond. The nucleic acid encoding the consensus NY-ESO-2 antigen can also include a nucleotide sequence encoding the IgE leader sequence. In some embodiments, the nucleic acid encoding the consensus NY-ESO-2 antigen is free of or does not contain a nucleotide sequence encoding the IgE leader sequence.

(9) PRAME

The vaccine of the present invention can comprise the cancer antigen PRAME, a fragment thereof, or a variant thereof. PRAME, encoded by the PRAME gene, is a protein comprised of 509 amino acids and is expressed in testis, placenta, endometrium, ovary, adrenals, and in tissues derived from melanoma, lung, kidney, and head and neck carcinomas. PRAME is also expressed in adult and pediatric acute leukemias, and multiple myeloma. PRAME contains an immunogenic nonapeptide able to elicit a cytotoxic response when presented by HLA-A24. Studies show that overexpression of PRAME in cultured cells induces a caspase-independent cell death responsible for a slower proliferation rate. Other studies demonstrate that overexpression of PRAME also confers growth or survival advantages by antagonizing retinoic acid receptor (RAR) signaling, and is causally involved in the tumorigenic process. Interference of RAR signaling leads to a loss in regulating cellular proliferation, development and differentiation.

PRAME can have an expression pattern similar to the cancer-testis antigens MAGE, BAGE, and GAGE, namely expression in the testis. PRAME, however, can be expressed in human melanomas and acute leukemias. PRAME can be recognized by cytolytic T lymphocytes. Accordingly, PRAME can be an antigen associated with melanoma and leukemias.

In one embodiment, the PRAME antigen is a canine PRAME antigen. In one embodiment, the PRAME antigen is a consensus PRAME antigen derived from multiple canine PRAME antigen sequences. In one embodiment, a PRAME antigen is operably linked to a signal peptide.

In one embodiment, a PRAME antigen can be administered in vectors described herein, and combined with the dTERT antigen and optionally one or more antibodies targeting one or more additional immune checkpoint proteins in various vaccination schedules.

The PRAME antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The PRAME antigen can comprise protein epitopes that make it particularly effective as an immunogen against which anti-PRAME immune responses can be induced. The PRAME antigen can comprise the full-length translation product, a variant thereof, a fragment thereof or a combination thereof. The PRAME antigen can comprise a consensus protein.

The nucleic acid sequence encoding the consensus PRAME antigen can be optimized with regards to codon usage and corresponding RNA transcripts. The nucleic acid encoding the consensus PRAME antigen can be codon and RNA optimized for expression in canines. In some embodiments, the nucleic acid sequence encoding the consensus PRAME antigen can include a Kozak sequence to increase the efficiency of translation. The nucleic acid encoding the consensus PRAME antigen can include one or more stop codons to increase the efficiency of translation termination.

The nucleic acid encoding the consensus PRAME antigen can also encode an immunoglobulin E (IgE) leader sequence. The nucleic acid encoding the consensus PRAME antigen can further encode the IgE leader sequence such that the amino acid sequence of the IgE leader sequence is linked to the amino acid sequence of the consensus PRAME antigen by a peptide bond. The nucleic acid encoding the consensus PRAME antigen can also include a nucleotide sequence encoding the IgE leader sequence. In some embodiments, the nucleic acid encoding the consensus PRAME antigen is free of or does not contain a nucleotide sequence encoding the IgE leader sequence.

(10) PSA

The vaccine of the present invention can comprise the cancer antigen prostate specific antigen (PSA; also known as gamma-seminoprotein or kallikrein-3 (KLK3)), a fragment thereof, or a variant thereof. PSA is an androgen-regulated serine protease produced by prostate epithelial cells and prostate cancer cells and encoded by the KLK3 gene. PSA is often used as a serum marker for prostate cancer. PSA is a member of the tissue kallikrein family and cleaves semenogelins in seminal coagulum after cleavage of the proenzyme to release the active enzyme, thereby liquefying semen to allow sperm to swim freely. Additionally, PSA enzymatic activity is regulated by zinc concentration, namely high zinc concentrations inhibit enzymatic activity of PSA.

In one embodiment, the PSA antigen is a canine PSA antigen. In one embodiment, the PSA antigen is a consensus PSA antigen derived from multiple canine PSA antigen sequences. In one embodiment, a PSA antigen is operably linked to a signal peptide.

In one embodiment, a PSA antigen can be administered in vectors described herein, and combined with the dTERT antigen and optionally one or more antibodies targeting one or more additional immune checkpoint proteins in various vaccination schedules.

The PSA antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The PSA antigen can comprise protein epitopes that make them particularly effective as immunogens against which anti-PSA immune responses can be induced. The PSA antigen can comprise the full-length translation product, a variant thereof, a fragment thereof or a combination thereof. The PSA antigen can comprise a consensus protein.

The nucleic acid sequence encoding the consensus PSA antigen can be optimized with regards to codon usage and corresponding RNA transcripts. The nucleic acid encoding the consensus PSA antigen can be codon and RNA optimized for expression in canines. In some embodiments, the nucleic acid sequence encoding the consensus PSA antigen can include a Kozak sequence to increase the efficiency of translation. The nucleic acid encoding the consensus PSA antigen can include one or more stop codons to increase the efficiency of translation termination.

The nucleic acid encoding the consensus PSA antigen can also encode an immunoglobulin E (IgE) leader sequence. The nucleic acid encoding the consensus PSA antigen can further encode the IgE leader sequence such that the amino acid sequence of the IgE leader sequence is linked to the amino acid sequence of the consensus PSA P antigen by a peptide bond. The nucleic acid encoding the consensus PSA antigen can also include a nucleotide sequence encoding the IgE leader sequence. In some embodiments, the nucleic acid encoding the consensus PSA antigen is free of or does not contain a nucleotide sequence encoding the IgE leader sequence.

In some embodiments, the nucleic acid encoding the consensus PSA antigen can be a heterologous nucleic acid sequence and/or contain one or more heterologous nucleic acid sequences.

(11) PSMA

The vaccine of the present invention can comprise the cancer antigen prostate specific membrane antigen (PSMA; also known as Glutamate carboxypeptidase II (GCPII), N-acetyl-L-aspartyl-L-glutamate peptidase I (NAALADase I), and NAAG peptidase), a fragment thereof, or a variant thereof. PSMA is encoded by the folate hydrolase 1 (FOLH1) gene. PSMA is a zinc metalloenzyme found residing in membranes and the extracellular space. PSMA is highly expressed in the human prostate and is upregulated in prostate cancer. PSMA is also found to be overexpressed in other cancers such as solid tumors of the kidney, breast, and colon.

In one embodiment, the PSMA antigen is a canine PSMA antigen. In one embodiment, the PSMA antigen is a consensus PSMA antigen derived from multiple canine PSMA antigen sequences. In one embodiment, a PSMA antigen is operably linked to a signal peptide.

In one embodiment, a PSMA antigen can be administered in vectors described herein, and combined with the dTERT antigen and optionally one or more antibodies targeting one or more additional immune checkpoint proteins in various vaccination schedules.

The PSMA antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The PSMA antigen can comprise protein epitopes that make them particularly effective as immunogens against which anti-PSMA immune responses can be induced. The PSMA antigen can comprise the full-length translation product, a variant thereof, a fragment thereof or a combination thereof. The PSMA antigen can comprise a consensus protein.

The nucleic acid sequence encoding the consensus PSMA antigen can be optimized with regards to codon usage and corresponding RNA transcripts. The nucleic acid encoding the consensus PSMA antigen can be codon and RNA optimized for expression in canines. In some embodiments, the nucleic acid sequence encoding the consensus PSMA antigen can include a Kozak sequence to increase the efficiency of translation. The nucleic acid encoding the consensus PSMA antigen can include one or more stop codons to increase the efficiency of translation termination.

The nucleic acid encoding the consensus PSMA antigen can also encode an immunoglobulin E (IgE) leader sequence. The nucleic acid encoding the consensus PSMA antigen can further encode the IgE leader sequence such that the amino acid sequence of the IgE leader sequence is linked to the amino acid sequence of the consensus PSMA antigen by a peptide bond. The nucleic acid encoding the consensus PSMA antigen can also include a nucleotide sequence encoding the IgE leader sequence. In some embodiments, the nucleic acid encoding the consensus PSMA antigen is free of or does not contain a nucleotide sequence encoding the IgE leader sequence.

In some embodiments, the nucleic acid encoding the consensus PSMA antigen can be a heterologous nucleic acid sequence and/or contain one or more heterologous nucleic acid sequences.

(12) STEAP

The vaccine of the present invention can comprise the cancer antigen six-transmembrane epithelial antigen of the prostate antigen (STEAP), a fragment thereof, or a variant thereof. STEAP is a metalloreductase encoded by the STEAP1 gene. STEAP is largely expressed in prostate tissues and is upregulated in cancer cells. STEAP is predicted to be a six-transmembrane protein and is a cell surface antigen found at cell-cell junctions.

In one embodiment, the STEAP antigen is a canine STEAP antigen. In one embodiment, the STEAP antigen is a consensus STEAP antigen derived from multiple canine STEAP antigen sequences. In one embodiment, a STEAP antigen is operably linked to a signal peptide.

In one embodiment, a STEAP antigen can be administered in vectors described herein, and combined with the dTERT antigen and optionally one or more antibodies targeting one or more additional immune checkpoint proteins in various vaccination schedules.

The STEAP antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The STEAP antigen can comprise protein epitopes that make them particularly effective as immunogens against which anti-STEAP immune responses can be induced. The STEAP antigen can comprise the full-length translation product, a variant thereof, a fragment thereof or a combination thereof. The STEAP antigen can comprise a consensus protein.

The nucleic acid sequence encoding the consensus STEAP antigen can be optimized with regards to codon usage and corresponding RNA transcripts. The nucleic acid encoding the consensus STEAP antigen can be codon and RNA optimized for expression in canines. In some embodiments, the nucleic acid sequence encoding the consensus STEAP antigen can include a Kozak sequence to increase the efficiency of translation. The nucleic acid encoding the consensus STEAP antigen can include one or more stop codons to increase the efficiency of translation termination.

The nucleic acid encoding the consensus STEAP antigen can also encode an immunoglobulin E (IgE) leader sequence. The nucleic acid encoding the consensus STEAP antigen can further encode the IgE leader sequence such that the amino acid sequence of the IgE leader sequence is linked to the amino acid sequence of the consensus STEAP antigen by a peptide bond. The nucleic acid encoding the consensus STEAP antigen can also include a nucleotide sequence encoding the IgE leader sequence. In some embodiments, the nucleic acid encoding the consensus STEAP antigen is free of or does not contain a nucleotide sequence encoding the IgE leader sequence.

In some embodiments, the nucleic acid encoding the consensus STEAP antigen can be a heterologous nucleic acid sequence and/or contain one or more heterologous nucleic acid sequences.

(13) PSCA

The vaccine of the present invention can comprise the cancer antigen prostate specific stem cell antigen (PSCA), a fragment thereof, or a variant thereof. PSCA is a glycosylphosphatidylinositol (GPI)-anchored cell surface protein and is encoded by an androgen-responsive gene. PSCA is a member of the Thy-1/Ly-6 family of GPI-anchored cell surface antigens. PSCA is upregulated in many cancers including prostate, bladder, and pancreatic cancers.

In one embodiment, the PSCA antigen is a canine PSCA antigen. In one embodiment, the PSCA antigen is a consensus PSCA antigen derived from multiple canine PSCA antigen sequences. In one embodiment, a PSCA antigen is operably linked to a signal peptide.

In one embodiment, a PSCA antigen can be administered in vectors described herein, and combined with the dTERT antigen and optionally one or more antibodies targeting one or more additional immune checkpoint proteins in various vaccination schedules.

The PSCA antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The PSCA antigen can comprise protein epitopes that make them particularly effective as immunogens against which anti-PSCA immune responses can be induced. The PSCA antigen can comprise the full-length translation product, a variant thereof, a fragment thereof or a combination thereof. The PSCA antigen can comprise a consensus protein.

The nucleic acid sequence encoding the consensus PSCA antigen can be optimized with regards to codon usage and corresponding RNA transcripts. The nucleic acid encoding the consensus PSCA antigen can be codon and RNA optimized for expression in canines. In some embodiments, the nucleic acid sequence encoding the consensus PSCA antigen can include a Kozak sequence to increase the efficiency of translation. The nucleic acid encoding the consensus PSCA antigen can include one or more stop codons to increase the efficiency of translation termination.

The nucleic acid encoding the consensus PSCA antigen can also encode an immunoglobulin E (IgE) leader sequence. The nucleic acid encoding the consensus PSCA antigen can further encode the IgE leader sequence such that the amino acid sequence of the IgE leader sequence is linked to the amino acid sequence of the consensus PSCA antigen by a peptide bond. The nucleic acid encoding the consensus PSCA antigen can also include a nucleotide sequence encoding the IgE leader sequence. In some embodiments, the nucleic acid encoding the consensus PSCA antigen is free of or does not contain a nucleotide sequence encoding the IgE leader sequence.

In some embodiments, the nucleic acid encoding the consensus PSCA antigen can be a heterologous nucleic acid sequence and/or contain one or more heterologous nucleic acid sequences.

(14) MAGE A1

The vaccine of the present invention can comprise the cancer antigen melanoma-associated antigen 1 (MAGE A1), a fragment thereof, or a variant thereof. MAGE A1, encoded by the MAGEA1 gene, is a 280-amino acid protein, and has been found only to be expressed by tumor cells and germ cells. MAGE A1 relies on DNA methylation for its repression in normal somatic tissues. These genes become activated in many types of tumors in the course of the genome-wide demethylation process, which often accompanies tumorigenesis. Specifically, during neoplastic transformation, these genes are activated, expressed, and may become antigenic targets that are recognized and attacked by the immune system. Therefore, MAGE genes take part in the immune process by targeting some early tumor cells for immune destruction. MAGE A1 may be expressed in numerous cancers, including, but not limited to, melanomas, lung carcinomas and esophageal squamous-cell carcinomas.

In one embodiment, the MAGE A1 antigen is a canine MAGE A1 antigen. In one embodiment, the MAGE A1 antigen is a consensus MAGE A1 antigen derived from multiple canine MAGE A1 antigen sequences. In one embodiment, a MAGE A1 antigen is operably linked to a signal peptide.

In one embodiment, a MAGE A1 antigen can be administered in vectors described herein, and combined with the dTERT antigen and optionally one or more antibodies targeting one or more additional immune checkpoint proteins in various vaccination schedules.

The MAGE A1 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

(15) WT1

The vaccine of the present invention can comprise the cancer antigen Wilm's tumor 1 (WT1), a fragment thereof, or a variant thereof. WT1 is a transcription factor containing at the N-terminus, a proline/glutamine-rich DNA-binding domain and at the C-terminus, four zinc finger motifs. WT1 plays a role in the normal development of the urogenital system and interacts with numerous factors, for example, p53, a known tumor suppressor and the serine protease HtrA2, which cleaves WT1 at multiple sites after treatment with a cytotoxic drug.

Mutation of WT1 can lead to tumor or cancer formation, for example, neproblastoma or tumors expressing WT1. Neproblastoma often forms in one or both kidneys before metastasizing to other tissues, for example, but not limited to, liver tissue, urinary tract system tissue, lymph tissue, and lung tissue. Accordingly, neproblastoma can be considered a metastatic tumor. Neproblastoma usually occurs in juvenile dogs.

In one embodiment, the WT1 antigen is a canine WT1 antigen. In one embodiment, the WT1 antigen is a consensus WT1 antigen derived from multiple canine WT1 antigen sequences. In one embodiment, a WT1 antigen is operably linked to a signal peptide.

In one embodiment, a WT1 antigen can be administered in vectors described herein, and combined with the dTERT antigen and optionally one or more antibodies targeting one or more additional immune checkpoint proteins in various vaccination schedules.

The WT-1 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

Accordingly, the vaccine can be used for treating subjects suffering from neproblastoma. The vaccine can be used for treating subjects suffering from any number of cancers including, but not limited to, melanoma, prostate cancer, liver cancer, cervical cancer, recurrent respiratory papillomatosis (RRP), anal cancer, head and neck cancer, and blood cancers. The vaccine can also be used for treating subjects with cancers or tumors that express WT1 for preventing development of such tumors in subjects. The WT1 antigen can differ from the native, “normal” WT1 gene, and thus, provide therapy or prophylaxis against an WT1 antigen-expressing tumor. Accordingly, WT1 antigen sequences that differ from the native WT1 gene (i.e., mutated WT1 genes or sequences) are provided herein.

The WT1 antigen can be a consensus antigen (or immunogen) sequence derived from two or more species. The WT1 antigen can comprise a consensus sequence and/or modification(s) for improved expression. Modification can include codon optimization, RNA optimization, additional of a kozak sequence for increased translation initiation and/or the addition of an immunoglobulin leader sequence to increase the immunogenicity of the WT1 antigen. The WT1 antigen can comprise a signal peptide such as an immunoglobulin signal peptide, for example, but not limited to, an immunoglobulin E (IgE) or immunoglobulin G (IgG) signal peptide. In some embodiments, the WT1 consensus antigen can comprise a hemagglutinin (HA) tag. The WT1 consensus antigen can be designed to elicit stronger and broader cellular and/or humoral immune responses than a corresponding codon optimized WT1 antigen.

(16) gp100

The vaccine of the present invention can comprise the cancer antigen glycoprotein 100 (gp100; also known as Trp2 and premelanosome protein (PMEL)), a fragment thereof, or a variant thereof. gp100 is encoded by the PMEL gene. It is a 70 kDa type 1 transmembrane glycoprotein, comprised of 661 amino acids that plays a central role in the biogenesis of melanosomes as it is involved in the maturation of melanosomes from stage I to II. gp100 drives the formation of striations from within multivesicular bodies and is directly involved in the biogenesis of premelanosomes. gp100 is enriched in premelanosomes relative to mature melanosomes, but overexpressed by proliferating neonatal melanocytes and during tumor growth. The gp100 protein includes a variety of immunogenic epitopes that are recognized by cytotoxic T lymphocytes from peripheral blood of melanoma patients and from tumor infiltrating lymphocytes.

In one embodiment, the GP100 antigen is a canine GP100 antigen. In one embodiment, the GP100 antigen is a consensus GP100 antigen derived from multiple canine GP100 antigen sequences. In one embodiment, a GP100 antigen is operably linked to a signal peptide.

In one embodiment, a GP100 antigen can be administered in vectors described herein, and combined with the dTERT antigen and optionally one or more antibodies targeting one or more additional immune checkpoint proteins in various vaccination schedules.

The gp100 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The gp100 antigen can comprise protein epitopes that make them particularly effective as immunogens against which anti-gp100 immune responses can be induced. The gp100 antigen can comprise the full-length translation product, a variant thereof, a fragment thereof or a combination thereof. The gp100 antigen can comprise a consensus protein.

The nucleic acid sequence encoding the consensus gp100 antigen can be optimized with regards to codon usage and corresponding RNA transcripts. The nucleic acid encoding the consensus gp100 antigen can be codon and RNA optimized for expression in canines. In some embodiments, the nucleic acid sequence encoding the consensus gp100 antigen can include a Kozak sequence to increase the efficiency of translation. The nucleic acid encoding the consensus gp100 antigen can include one or more stop codons to increase the efficiency of translation termination.

The nucleic acid encoding the gp100 antigen can also encode an immunoglobulin E (IgE) leader sequence. The nucleic acid encoding the gp100 antigen can further encode the IgE leader sequence such that the amino acid sequence of the IgE leader sequence is linked to the amino acid sequence of the gp100 antigen by a peptide bond. The nucleic acid encoding the gp100 antigen can also include a nucleotide sequence encoding the IgE leader sequence. In some embodiments, the nucleic acid encoding the gp100 antigen is free of or does not contain a nucleotide sequence encoding the IgE leader sequence.

(17) FSHR

Follicle stimulating hormone receptor (FSHR) is an antigen that is selectively expressed in females in the ovarian granulosa cells (Simoni et al., Endocr Rev. 1997, 18:739-773) and at low levels in the ovarian endothelium (Vannier et al., Biochemistry, 1996, 35:1358-1366).

In various embodiments, the FSHR antigen comprises a consensus protein or a nucleic acid molecule encoding a consensus protein. FSHR antigens include sequences homologous to the FSHR antigens, fragments of the FSHR antigens and proteins with sequences homologous to fragments of the FSHR antigens.

The FSHR antigens can be administered in vectors described herein, and combined with the CTLA4 antibody and optionally one or more antibodies targeting one or more additional immune checkpoint proteins in various vaccination schedules.

In one embodiment, an FSHR antigen is canine FSHR. In one embodiment, an FSHR antigen is a consensus FSHR antigen derived from multiple canine FSHR sequences. In one embodiment, a FSHR antigen is operably linked to a signal peptide.

In one embodiment, an FSHR antigen can be administered in vectors described herein, and combined with the dTERT antigen and optionally one or more antibodies targeting one or more additional immune checkpoint proteins in various vaccination schedules.

The FSHR antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The FSHR antigen can comprise protein epitopes that make them particularly effective as immunogens against which anti-FSHR immune responses can be induced. The FSHR antigen can comprise the full-length translation product, a variant thereof, a fragment thereof or a combination thereof. The FSHR antigen can comprise a consensus protein.

The nucleic acid sequence encoding the consensus FSHR antigen can be optimized with regards to codon usage and corresponding RNA transcripts. The nucleic acid encoding the consensus FSHR antigen can be codon and RNA optimized for expression in canines. In some embodiments, the nucleic acid sequence encoding the consensus FSHR antigen can include a Kozak sequence to increase the efficiency of translation. The nucleic acid encoding the consensus FSHR antigen can include one or more stop codons to increase the efficiency of translation termination.

The nucleic acid encoding the FSHR antigen can also encode an immunoglobulin E (IgE) leader sequence. The nucleic acid encoding the FSHR antigen can further encode the IgE leader sequence such that the amino acid sequence of the IgE leader sequence is linked to the amino acid sequence of the FSHR antigen by a peptide bond. The nucleic acid encoding the FSHR antigen can also include a nucleotide sequence encoding the IgE leader sequence. In some embodiments, the nucleic acid encoding the FSHR antigen is free of or does not contain a nucleotide sequence encoding the IgE leader sequence.

(18) Tumor Microenvironment Antigens

Several proteins are overexpressed in the tumor microenvironment including, but not limited to, Fibroblast Activation Protein (FAP), Platelet Derived Growth Factor Receptor Beta (PDGFR-β), and Glypican-1 (GPC1). FAP is a membrane-bound enzyme with gelatinase and peptidase activity that is up-regulated in cancer-associated fibroblasts in over 90% of human carcinomas. PDGFR-β is a cell surface tyrosine kinase receptor that has roles in the regulation of many biological processes including embryonic development, angiogenesis, cell proliferation and differentiation. GPC1 is a cell surface proteoglycan that is enriched in cancer cells.

In various embodiments, the tumor microenvironment antigen comprises a consensus protein or a nucleic acid molecule encoding a consensus protein. Tumor microenvironment antigens include sequences homologous to the tumor microenvironment antigens, fragments of the tumor microenvironment antigens and proteins with sequences homologous to fragments of the tumor microenvironment antigens.

In one embodiment, the tumor microenvironment antigen is a canine tumor microenvironment antigen. In one embodiment, the tumor microenvironment antigen is a consensus tumor microenvironment antigen derived from multiple canine tumor microenvironment antigen sequences. In one embodiment, a tumor microenvironment antigen is operably linked to a signal peptide.

One or more tumor microenvironment antigens can be administered in vectors described herein, and combined with the dTERT antigen and optionally one or more antibodies targeting one or more additional immune checkpoint proteins in various vaccination schedules.

The tumor microenvironment antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The tumor microenvironment antigen can comprise protein epitopes that make them particularly effective as immunogens against which anti-tumor microenvironment immune responses can be induced. The tumor microenvironment antigen can comprise the full-length translation product, a variant thereof, a fragment thereof or a combination thereof. The tumor microenvironment antigen can comprise a consensus protein.

The nucleic acid sequence encoding the consensus tumor microenvironment antigen can be optimized with regards to codon usage and corresponding RNA transcripts. The nucleic acid encoding the consensus tumor microenvironment antigen can be codon and RNA optimized for expression in canines. In some embodiments, the nucleic acid sequence encoding the consensus tumor microenvironment antigen can include a Kozak sequence to increase the efficiency of translation. The nucleic acid encoding the consensus tumor microenvironment antigen can include one or more stop codons to increase the efficiency of translation termination.

The nucleic acid encoding the tumor microenvironment antigen can also encode an immunoglobulin E (IgE) leader sequence. The nucleic acid encoding the tumor microenvironment antigen can further encode the IgE leader sequence such that the amino acid sequence of the IgE leader sequence is linked to the amino acid sequence of the tumor microenvironment antigen by a peptide bond. The nucleic acid encoding the tumor microenvironment antigen can also include a nucleotide sequence encoding the IgE leader sequence. In some embodiments, the nucleic acid encoding the tumor microenvironment antigen is free of or does not contain a nucleotide sequence encoding the IgE leader sequence.

(19) Viral Antigens

The cancer antigen can be a viral antigen, a fragment thereof, or a variant thereof. The viral antigen can be antigen from a canine virus (e.g., Canine Papillomavirus or Canine EBV-like virus). In one embodiment, the viral antigen is a consensus viral antigen derived from multiple canine virus antigen sequences. In one embodiment, a viral antigen is operably linked to a signal peptide.

In one embodiment, the viral antigen can be administered in vectors described herein, and combined with the dTERT antigen and optionally one or more antibodies targeting one or more additional immune checkpoint proteins in various vaccination schedules.

The viral antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune response. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts, soluble factors produced by immune suppressor cells, CTLA-4, PD-1, MDSCs, MCP-1, and an immune checkpoint molecule, which is described below in more detail.

The viral antigen can comprise protein epitopes that make them particularly effective as immunogens against which anti-viral immune responses can be induced. The viral antigen can comprise the full-length translation product, a variant thereof, a fragment thereof or a combination thereof. The viral antigen can comprise a consensus protein.

The nucleic acid sequence encoding the viral antigen can be optimized with regards to codon usage and corresponding RNA transcripts. The nucleic acid encoding the viral antigen can be codon and RNA optimized for expression in canines. In some embodiments, the nucleic acid sequence encoding the viral antigen can include a Kozak sequence to increase the efficiency of translation. The nucleic acid encoding the viral antigen can include one or more stop codons to increase the efficiency of translation termination.

The nucleic acid encoding the viral antigen can also encode an immunoglobulin E (IgE) leader sequence. The nucleic acid encoding the viral antigen can further encode the IgE leader sequence such that the amino acid sequence of the IgE leader sequence is linked to the amino acid sequence of the viral antigen by a peptide bond. The nucleic acid encoding the viral antigen can also include a nucleotide sequence encoding the IgE leader sequence. In some embodiments, the nucleic acid encoding the viral antigen is free of or does not contain a nucleotide sequence encoding the IgE leader sequence.

### 3. VACCINE IN COMBINATION WITH IMMUNE CHECKPOINT INHIBITOR

The vaccine can further comprise one or more inhibitors of one or more immune checkpoint molecules (i.e., an immune checkpoint inhibitor). Immune checkpoint molecules are described below in more detail. The immune checkpoint inhibitor is any nucleic acid or protein that prevents the suppression of any component in the immune system such as MHC class presentation, T cell presentation and/or differentiation, B cell presentation and/or differentiation, any cytokine, chemokine or signaling for immune cell proliferation and/or differentiation.

Such an inhibitor can be a nucleic acid sequence, an amino acid sequence, a small molecule, or a combination thereof. The nucleic acid sequence can be DNA, RNA, cDNA, a variant thereof, a fragment thereof, or a combination thereof. The nucleic acid can also include additional sequences that encode linker or tag sequences that are linked to the immune checkpoint inhibitor by a peptide bond. The small molecule may be a low molecular weight, for example, less than 800 Daltons, organic or inorganic compound that can serve as an enzyme substrate, ligand (or analog thereof) bound by a protein or nucleic acid, or regulator of a biological process. The amino acid sequence can be protein, a peptide, a variant thereof, a fragment thereof, or a combination thereof.

In some embodiments, the immune checkpoint inhibitor can be one or more nucleic acid sequences encoding an antibody, a variant thereof, a fragment thereof, or a combination thereof. In other embodiments, the immune checkpoint inhibitor can be an antibody, a variant thereof, a fragment thereof, or a combination thereof.

a. Immune Checkpoint Molecule

The immune checkpoint molecule can be a nucleic acid sequence, an amino acid sequence, a small molecule, or a combination thereof. The nucleic acid sequence can be DNA, RNA, cDNA, a variant thereof, a fragment thereof, or a combination thereof. The nucleic acid can also include additional sequences that encode linker or tag sequences that are linked to the immune checkpoint inhibitor by a peptide bond. The small molecule may be a low molecular weight, for example, less than 800 Daltons, organic or inorganic compound that can serve as an enzyme substrate, ligand (or analog thereof) bound by a protein or nucleic acid, or regulator of a biological process. The amino acid sequence can be protein, a peptide, a variant thereof, a fragment thereof, or a combination thereof.

(1) PD-1 and PD-L1

The immune checkpoint molecule may programmed cell death protein 1 (PD-1), programmed cell death ligand 1 (PD-L1), a fragment thereof, a variant thereof, or a combination thereof. PD-1 is a cell surface protein encoded by the PDCD1 gene. PD-1 is a member of the immunoglobulin superfamily and is expressed on T cells and pro-B cells, and thus, contributes to the fate and/or differentiation of these cells. In particular, PD-1 is a type 1 membrane protein of the CD28/CTLA-4 family of T cell regulators and negatively regulates T cell receptor (TCR) signals, thereby negatively regulating immune responses. PD-1 can negatively regulate CD8+ T cell responses, and thus inhibit CD8-mediated cytotoxicity and enhance tumor growth.

PD-1 has two ligands, PD-L1 and PD-L2, which are members of the B7 family. PD-L1 is upregulated on macrophages and dendritic cells (DCs) in response to LPS and GM-CSF treatment and on T cells and B cells upon TCR and B cell receptor signaling. PD-L1 is expressed by many tumor cell lines, including myelomas, mastocytomas, and melanomas.

b. Anti-Immune Checkpoint Molecule Antibody

As described above, the immune checkpoint inhibitor can be an antibody. The antibody can bind or react with an antigen (i.e., the immune checkpoint molecule described above). Accordingly, the antibody may be considered an anti-immune checkpoint molecule antibody or an immune checkpoint molecule antibody. The antibody can be encoded by a nucleic acid sequence.

The antibody can include a heavy chain polypeptide and a light chain polypeptide. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

The light chain polypeptide can include a variable light chain (VL) region and/or a constant light chain (CL) region. The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

Additionally, the proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

(1) PD-1 Antibody

The anti-immune checkpoint molecule antibody can be an anti-PD-1 antibody (also referred to herein as “PD-1 antibody”), a variant thereof, a fragment thereof, or a combination thereof. The PD-1 antibody can be Nivolumab. The anti-PD-1 antibody can inhibit PD-1 activity, thereby inducing, eliciting, or increasing an immune response against a tumor or cancer and decreasing tumor growth.

(2) PD-L1 Antibody

The anti-immune checkpoint molecule antibody can be an anti-PD-L1 antibody (also referred to herein as “PD-L1 antibody”), a variant thereof, a fragment thereof, or a combination thereof. The anti-PD-L1 antibody can inhibit PD-L1 activity, thereby inducing, eliciting, or increasing an immune response against a tumor or cancer and decreasing tumor growth.

### 4. VACCINE CONSTRUCTS AND PLASMIDS

The vaccine can comprise nucleic acid constructs or plasmids that encode the above described antigens and/or antibodies. The nucleic acid constructs or plasmids can include or contain one or more heterologous nucleic acid sequences. Provided herein are genetic constructs that can comprise a nucleic acid sequence that encodes the above described antigens and/or antibodies. The genetic construct can be present in the cell as a functioning extrachromosomal molecule. The genetic construct can be a linear minichromosome including centromere, telomeres or plasmids or cosmids. The genetic constructs can include or contain one or more heterologous nucleic acid sequences.

The genetic constructs can be in the form of plasmids expressing the above described antigens and/or antibodies in any order.

The genetic construct can also be part of a genome of a recombinant viral vector, including recombinant adenovirus, recombinant adenovirus associated virus and recombinant vaccinia. The genetic construct can be part of the genetic material in attenuated live microorganisms or recombinant microbial vectors which live in cells.

The genetic constructs can comprise regulatory elements for gene expression of the coding sequences of the nucleic acid. The regulatory elements can be a promoter, an enhancer an initiation codon, a stop codon, or a polyadenylation signal.

The nucleic acid sequences can make up a genetic construct that can be a vector. The vector can be capable of expressing the above described antigens and/or antibodies in the cell of a mammal in a quantity effective to elicit an immune response in the mammal. The vector can be recombinant. The vector can comprise heterologous nucleic acid encoding the the above described antigens and/or antibodies. The vector can be a plasmid. The vector can be useful for transfecting cells with nucleic acid encoding the above described antigens and/or antibodies, which the transformed host cell is cultured and maintained under conditions wherein expression of the above described antigens and/or antibodies takes place.

Coding sequences can be optimized for stability and high levels of expression. In some instances, codons are selected to reduce secondary structure formation of the RNA such as that formed due to intramolecular bonding.

The vector can comprise heterologous nucleic acid encoding the above described antigens and/or antibodies and can further comprise an initiation codon, which can be upstream of the one or more cancer antigen coding sequence(s), and a stop codon, which can be downstream of the coding sequence(s) of the above described antigens and/or antibodies. The initiation and termination codon can be in frame with the coding sequence(s) of the above described antigens and/or antibodies. The vector can also comprise a promoter that is operably linked to the coding sequence(s) of the above described antigens and/or antibodies. The promoter operably linked to the coding sequence(s) of the above described antigens and/or antibodies can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene such as human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the contents of which are incorporated herein in its entirety.

The vector can also comprise a polyadenylation signal, which can be downstream of the coding sequence(s) of the above described antigens and/or antibodies. The polyadenylation signal can be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal can be a polyadenylation signal from a pCEP4 vector (Invitrogen, San Diego, Calif.).

The vector can also comprise an enhancer upstream of the above described antigens and/or antibodies. The enhancer can be necessary for DNA expression. The enhancer can be human actin, human myosin, human hemoglobin, human muscle creatine or a viral enhancer such as one from CMV, HA, RSV or EBV. Polynucleotide function enhancers are described in U.S. Pat. Nos. 5,593,972, 5,962,428, and WO94/016737, the contents of each are fully incorporated by reference.

The vector can also comprise a mammalian origin of replication in order to maintain the vector extrachromosomally and produce multiple copies of the vector in a cell. The vector can be pVAX1, pCEP4 or pREP4 from Invitrogen (San Diego, Calif.), which can comprise the Epstein Barr virus origin of replication and nuclear antigen EBNA-1 coding region, which can produce high copy episomal replication without integration. The vector can be pVAX1 or a pVax1 variant with changes such as the variant plasmid described herein. The variant pVax1 plasmid is a 2998 basepair variant of the backbone vector plasmid pVAX1 (Invitrogen, Carlsbad Calif.). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993.

The backbone of the vector can be pAV0242. The vector can be a replication defective adenovirus type 5 (Ad5) vector.

The vector can also comprise a regulatory sequence, which can be well suited for gene expression in a mammalian or canine cell into which the vector is administered. The one or more cancer antigen sequences disclosed herein can comprise a codon, which can allow more efficient transcription of the coding sequence in the host cell.

The vector can be pSE420 (Invitrogen, San Diego, Calif.), which can be used for protein production in Escherichia coli (E. coli). The vector can also be pYES2 (Invitrogen, San Diego, Calif.), which can be used for protein production in Saccharomyces cerevisiae strains of yeast. The vector can also be of the MAXBAC™ complete baculovirus expression system (Invitrogen, San Diego, Calif.), which can be used for protein production in insect cells. The vector can also be pcDNA I or pcDNA3 (Invitrogen, San Diego, Calif.), which may be used for protein production in mammalian cells, such as Chinese hamster ovary (CHO) cells. The vector can be expression vectors or systems to produce protein by routine techniques and readily available starting materials including Sambrook et al., Molecular Cloning and Laboratory Manual, Second Ed., Cold Spring Harbor (1989), which is incorporated fully by reference.

In some embodiments, the vector can comprise one or more of the nucleic acid sequences of SEQ ID NOs: 1, and/or 3, or a fragment or variant thereof.

### 5. PHARMACEUTICAL COMPOSITIONS OF THE VACCINE

The vaccine can be in the form of a pharmaceutical composition. The pharmaceutical composition can comprise the vaccine. The pharmaceutical compositions can comprise about 5 nanograms to about 10 mg of the DNA of the vaccine. In some embodiments, pharmaceutical compositions according to the present invention comprise about 25 nanogram to about 5 mg of DNA of the vaccine. In some embodiments, the pharmaceutical compositions contain about 50 nanograms to about 1 mg of DNA of the vaccine. In some embodiments, the pharmaceutical compositions contain about 0.1 to about 500 micrograms of DNA of the vaccine. In some embodiments, the pharmaceutical compositions contain about 1 to about 350 micrograms of DNA of the vaccine. In some embodiments, the pharmaceutical compositions contain about 5 to about 250 micrograms of DNA of the vaccine. In some embodiments, the pharmaceutical compositions contain about 10 to about 200 micrograms of DNA of the vaccine. In some embodiments, the pharmaceutical compositions contain about 15 to about 150 micrograms of DNA of the vaccine. In some embodiments, the pharmaceutical compositions contain about 20 to about 100 micrograms of DNA of the vaccine. In some embodiments, the pharmaceutical compositions contain about 25 to about 75 micrograms of DNA of the vaccine. In some embodiments, the pharmaceutical compositions contain about 30 to about 50 micrograms of DNA of the vaccine. In some embodiments, the pharmaceutical compositions contain about 35 to about 40 micrograms of DNA of the vaccine. In some embodiments, the pharmaceutical compositions contain about 100 to about 200 micrograms of DNA of the vaccine. In some embodiments, the pharmaceutical compositions comprise about 10 micrograms to about 100 micrograms of DNA of the vaccine. In some embodiments, the pharmaceutical compositions comprise about 20 micrograms to about 80 micrograms of DNA of the vaccine. In some embodiments, the pharmaceutical compositions comprise about 25 micrograms to about 60 micrograms of DNA of the vaccine. In some embodiments, the pharmaceutical compositions comprise about 30 nanograms to about 50 micrograms of DNA of the vaccine. In some embodiments, the pharmaceutical compositions comprise about 35 nanograms to about 45 micrograms of DNA of the vaccine. In some embodiments, the pharmaceutical compositions contain about 0.1 to about 500 micrograms of DNA of the vaccine. In some embodiments, the pharmaceutical compositions contain about 1 to about 350 micrograms of DNA of the vaccine. In some embodiments, the pharmaceutical compositions contain about 25 to about 250 micrograms of DNA of the vaccine. In some embodiments, the pharmaceutical compositions contain about 100 to about 200 microgram DNA of the vaccine.

In some embodiments, pharmaceutical compositions according to the present invention comprise at least 10, 15, 20, 25, 30, 35, 40, 45, 50, 55, 60, 65, 70, 75, 80, 85, 90, 95 or 100 nanograms of DNA of the vaccine. In some embodiments, the pharmaceutical compositions can comprise at least 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995 or 1000 micrograms of DNA of the vaccine. In some embodiments, the pharmaceutical composition can comprise at least 1.5, 2, 2.5, 3, 3.5, 4, 4.5, 5, 5.5, 6, 6.5, 7, 7.5, 8, 8.5, 9, 9.5 or 10 mg or more of DNA of the vaccine.

In other embodiments, the pharmaceutical composition can comprise up to and including 15, 20, 25, 30, 35, 40, 45, 50, 55, 60, 65, 70, 75, 80, 85, 90, 95 or 100 nanograms of DNA of the vaccine. In some embodiments, the pharmaceutical composition can comprise up to and including 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995, or 1000 micrograms of DNA of the vaccine. In some embodiments, the pharmaceutical composition can comprise up to and including 1.5, 2, 2.5, 3, 3.5, 4, 4.5, 5, 5.5, 6, 6.5, 7, 7.5, 8, 8.5, 9, 9.5 or 10 mg of DNA of the vaccine.

The pharmaceutical composition can further comprise other agents for formulation purposes according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The vaccine can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent may be a polyanion, polycation, including poly-L-glutamate (LGS), or lipid. The transfection facilitating agent may be poly-L-glutamate, and for example, the poly-L-glutamate is present in the vaccine at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid can also be used administered in conjunction with the genetic construct. In some embodiments, the DNA vector vaccines can also include a transfection facilitating agent such as lipids, liposomes, including lecithin liposomes or other liposomes known in the art, as a DNA-liposome mixture (see for example WO1993024640A2), calcium ions, viral proteins, polyanions, polycations, or nanoparticles, or other known transfection facilitating agents. Preferably, the transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can include an adjuvant. The adjuvant can be other genes that are expressed in a plasmid or are delivered as proteins in combination with the plasmid above in the vaccine. The adjuvant can be selected from α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof. In an exemplary embodiment, the adjuvant is IL-12.

Other genes which can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and/or functional fragments thereof.

### 6. COMBINATIONAL VACCINES FOR TREATING PARTICULAR CANCERS

The vaccine can be in the form of various combinations of the cancer antigens as described above to treat particular cancers or tumors. Depending upon the combination of one or more cancer antigens, various cancers or other tumor types may be targeted with the vaccine. These cancers can include melanoma, blood cancers (e.g., leukemia, lymphoma, myeloma), lung carcinomas, esophageal squamous cell carcinomas, bladder cancer, colorectal cancer, esophagus, gastric cancer, hepatocarcinoma, head and neck, brain, anal cancer, non-small cell lung carcinoma, pancreatic cancer, synovial carcinoma, prostate cancer, testicular cancer, liver cancer, cervical cancer, recurrent respiratory papillomatosis, skin cancer and stomach cancer.

a. Melanoma

The vaccine can combine one or more cancer antigens such as tyrosinase, PRAME, or GP100-Trp2 to treat or prevent melanoma. The vaccine can further combine one or more of cancer antigen tyrosinase, PRAME, and GP100-Trp2 with any one or more cancer antigens dTERT, NY-ESO-1, MAGE-A1, and WT1 for treating or preventing melanoma. Other combinations of cancer antigens may also be applied for treating or preventing melanoma.

b. Head and Neck Cancer

The vaccine can comprise cancer antigen HPV 16 E6/E7 to treat or prevent head and neck cancer. The vaccine can further combine cancer antigen HPV 16 E6/E7 with any one or more of cancer antigens dTERT, NY-ESO-1, MAGE-A1, and WT1 for treating or preventing head and neck cancer. Other combinations of cancer antigens may also be applied for treating or preventing head and neck cancer.

c. Recurrent Respiratory Papillomatosis/Anal Cancer

The vaccine can combine one or more cancer antigens such as HPV 6, HPV11, and HPV 16 to treat or prevent recurrent respiratory papillomatosis and/or anal cancer. The vaccine can further combine one or more cancer antigens HPV 6, HPV11 and HPV16 with one or more cancer antigens dTERT, NY-ESO-1, MAGE-A1, and WT1 for treating or preventing recurrent respiratory papilloatosis and/or anal cancer. Other combinations of cancer antigens may also be applied for treating or preventing recurrent respiratory papilloatosis and/or anal cancer.

d. Cervical Cancer

The vaccine can combine one or more cancer antigens such as HPV 16 E6/E7 and HPV 18 E6/E7 to treat or prevent cervical cancer. The vaccine can further combine one or more cancer antigens such as HPV 16 E6/E7 and HPV 18 E6/E7 with one or more cancer antigens such as dTERT, NY-ESO-1, MAGE-A1, and WT1 for treating or preventing cervical cancer. Other combinations of cancer antigens may also be applied for treating or preventing cervical cancer.

e. Liver Cancer

The vaccine can combine one or more cancer antigens such as HBV core antigen, HBV surface antigen, HCVNS34A, HCVNS5A, HCV NS5B, and HCVNS4B to treat or prevent liver cancer. The vaccine can further combine one or more cancer antigens HBV core antigen, HBV surface antigen, HCVNS34A, HCVNS5A, HCV NS5B, and HCVNS4B with one or more of cancer antigens dTERT, NY-ESO-1, MAGE-A1, and WT1 for treating or preventing liver cancer. Other combinations of cancer antigens may also be applied for treating or preventing liver cancer.

f. Glioblastoma

The vaccine can comprise CMV to treat or prevent glioblastoma. The vaccine can further combine CMV with one or more of cancer antigens dTERT, NY-ESO-1, MAGE-A1, or WT1 for treating or preventing glioblastoma. Other combinations of cancer antigens may also be applied for treating or preventing glioblastoma.

g. Prostate

The vaccine can combine one or more cancer antigens such as PSA, PSMA, and STEAP to treat or prevent prostate cancer. The vaccine can further combine one or more cancer antigens PSA, PSMA, and STEAP with one or more of cancer antigens dTERT, NY-ESO-1, MAGE-A1, and WT1 for treating or preventing prostate cancer. Other combinations of cancer antigens may also be applied for treating or preventing prostate cancer.

h. Blood Cancers (e.g., Leukemia, Lymphoma, Myeloma)

The vaccine can combine one or more cancer antigens such as PRAME, WT-1, and dTERT to treat or prevent blood cancers such as leukemia, lymphoma and myeloma. The vaccine can further combine one or more cancer antigens PRAME, WT-1, and dTERT with one or more of cancer antigens NY-ESO-1, and MAGE-A1 for treating or preventing blood cancers such as leukemia, lymphoma and myeloma. Other combinations of cancer antigens may also be applied for treating or preventing blood cancers such as leukemia, lymphoma and myeloma cancer.

### 7. METHOD OF VACCINATION

Provided herein is a method for treating or preventing cancer using the pharmaceutical formulations for providing genetic constructs and proteins of the one or more cancer antigens as described above, which comprise epitopes that make them particularly effective immunogens against which an immune response to the one or more cancer antigens can be induced. The method of administering the vaccine, or vaccination, can be provided to induce a therapeutic and/or prophylactic immune response. The vaccination process can generate in the mammal an immune response against one or more of the cancer antigens as disclosed herein. The vaccine can be administered to an individual to modulate the activity of the mammal's immune system and enhance the immune response. The administration of the vaccine can be the transfection of the one or more cancer antigens as disclosed herein as a nucleic acid molecule that is expressed in the cell and thus, delivered to the surface of the cell upon which the immune system recognizes and induces a cellular, humoral, or cellular and humoral response. The administration of the vaccine can be used to induce or elicit an immune response in mammals against one or more of the cancer antigens as disclosed herein by administering to the mammals the vaccine as discussed herein.

Upon administration of the vaccine to the mammal, and thereupon the vector into the cells of the mammal, the transfected cells will express and secrete one or more of the cancer antigens as disclosed herein. These secreted proteins, or synthetic antigens, will be recognized as foreign by the immune system, which will mount an immune response that can include antibodies made against the one or more cancer antigens, and a T-cell response specifically against the one or more cancer antigens. In some examples, a mammal vaccinated with the vaccines discussed herein will have a primed immune system and when challenged with the one or more cancer antigens as disclosed herein, the primed immune system will allow for rapid clearing of subsequent cancer antigens as disclosed herein, whether through the humoral, cellular, or both cellular and humoral immune response. The vaccine can be administered to an individual to modulate the activity of the individual's immune system, thereby enhancing the immune response.

Methods of administering the DNA of a vaccine are described in U.S. Pat. Nos. 4,945,050 and 5,036,006, both of which are incorporated herein in their entirety by reference.

The vaccine can be administered to a mammal to elicit an immune response in a mammal. The mammal can be human, non-human primate, cow, pig, sheep, goat, antelope, bison, water buffalo, bovids, deer, hedgehogs, elephants, llama, alpaca, mice, rats, or chicken. In an exemplary embodiment, the vaccine can be administered to a dog.

The vaccine dose can be between 1 μg to 10 mg active component/kg body weight/time and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, 10, or more doses.

a. Method of Generating an Immune Response with the Vaccine

The vaccine can be used to generate an immune response in a mammal, including a therapeutic or prophylactic immune response. The immune response can generate antibodies and/or killer T cells which are directed to the one or more cancer antigens as disclosed herein. Such antibodies and T cells can be isolated.

Some embodiments provide methods of generating immune responses against one or more of the cancer antigens as disclosed herein, which comprise administering to an individual the vaccine. Some embodiments provide methods of prophylactically vaccinating an individual against a cancer or tumor expressing one or more of the cancer antigens as described above, which comprise administering the vaccine. Some embodiments provide methods of therapeutically vaccinating an individual that has been suffering from the cancer or tumor expressing one or more of the cancer antigens, which comprise administering the vaccine. Diagnosis of the cancer or tumor expressing the one or more cancer antigens as disclosed herein prior to administration of the vaccine can be performed routinely.

b. Method of Cancer Treatment with the Vaccine

The vaccine can be used to generate or elicit an immune response in a mammal that is reactive or directed to a cancer or tumor (e.g., melanoma, head and neck, cervical, liver, prostate, blood cancers, esophageal squamous, gastric) of the mammal or subject in need thereof. The elicited immune response can prevent cancer or tumor growth.

The elicited immune response can prevent and/or reduce metastasis of cancerous or tumor cells. Accordingly, the vaccine can be used in a method that treats and/or prevents cancer or tumors in the mammal or subject administered the vaccine. Depending upon the antigen used in the vaccine, the treated cancer or tumor based growth can be any type of cancer such as, but not limited to, melanoma, blood cancers (e.g., leukemia, lymphoma, myeloma), lung carcinomas, esophageal squamous cell carcinomas, bladder cancer, colorectal cancer, esophagus, gastric cancer, hepatocarcinoma, head and neck, brain, anal cancer, non-small cell lung carcinoma, pancreatic cancer, synovial carcinoma, prostate cancer, testicular cancer, liver cancer, cervical cancer, recurrent respiratory papillomatosis, skin cancer and stomach cancer.

In some embodiments, the administered vaccine can mediate clearance or prevent growth of tumor cells by inducing (1) humoral immunity via B cell responses to generate antibodies that block monocyte chemoattractant protein-1 (MCP-1) production, thereby retarding myeloid derived suppressor cells (MDSCs) and suppressing tumor growth; (2) increase cytotoxic T lymphocyte such as CD8⁺ (CTL) to attack and kill tumor cells; (3) increase T helper cell responses; (4) and increase inflammatory responses via IFN-γ and TFN-α or preferably all of the aforementioned.

In some embodiments, the immune response can generate a humoral immune response and/or an antigen-specific cytotoxic T lymphocyte (CTL) response that does not cause damage to or inflammation of various tissues or systems (e.g., brain or neurological system, etc.) in the subject administered the vaccine.

In some embodiments, the administered vaccine can increase tumor free survival, reduce tumor mass, increase tumor survival, or a combination thereof in the subject. The administered vaccine can increase tumor free survival by 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, and 60% or more in the subject. The administered vaccine can reduce tumor mass by 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, and 70% or more in the subject after immunization. The administered vaccine can prevent and block increases in monocyte chemoattractant protein 1 (MCP-1), a cytokine secreted by myeloid derived suppressor cells, in the subject. In some embodiments, the administered vaccine can prevent and block increases in MCP-1 within the cancerous or tumor tissue in the subject, thereby reducing vascularization of the cancerous or tumor tissue in the subject.

The administered vaccine can increase tumor survival by 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, and 70% or more in the subject. In some embodiments, the vaccine can be administered to the periphery (as described in more detail below) to establish an antigen-specific immune response targeting the cancerous or tumor cells or tissue to clear or eliminate the cancer or tumor expressing the one or more cancer antigens without damaging or causing illness or death in the subject administered the vaccine.

The administered vaccine can increase a cellular immune response in the subject by about 50-fold to about 6000-fold, about 50-fold to about 5500-fold, about 50-fold to about 5000-fold, about 50-fold to about 4500-fold, about 100-fold to about 6000-fold, about 150-fold to about 6000-fold, about 200-fold to about 6000-fold, about 250-fold to about 6000-fold, or about 300-fold to about 6000-fold. In some embodiments, the administered vaccine can increase the cellular immune response in the subject by about 50-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, 1000-fold, 1100-fold, 1200-fold, 1300-fold, 1400-fold, 1500-fold, 1600-fold, 1700-fold, 1800-fold, 1900-fold, 2000-fold, 2100-fold, 2200-fold, 2300-fold, 2400-fold, 2500-fold, 2600-fold, 2700-fold, 2800-fold, 2900-fold, 3000-fold, 3100-fold, 3200-fold, 3300-fold, 3400-fold, 3500-fold, 3600-fold, 3700-fold, 3800-fold, 3900-fold, 4000-fold, 4100-fold, 4200-fold, 4300-fold, 4400-fold, 4500-fold, 4600-fold, 4700-fold, 4800-fold, 4900-fold, 5000-fold, 5100-fold, 5200-fold, 5300-fold, 5400-fold, 5500-fold, 5600-fold, 5700-fold, 5800-fold, 5900-fold, or 6000-fold.

The administered vaccine can increase interferon gamma (IFN-γ) levels in the subject by about 50-fold to about 6000-fold, about 50-fold to about 5500-fold, about 50-fold to about 5000-fold, about 50-fold to about 4500-fold, about 100-fold to about 6000-fold, about 150-fold to about 6000-fold, about 200-fold to about 6000-fold, about 250-fold to about 6000-fold, or about 300-fold to about 6000-fold. In some embodiments, the administered vaccine can increase IFN-γ levels in the subject by about 50-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, 1000-fold, 1100-fold, 1200-fold, 1300-fold, 1400-fold, 1500-fold, 1600-fold, 1700-fold, 1800-fold, 1900-fold, 2000-fold, 2100-fold, 2200-fold, 2300-fold, 2400-fold, 2500-fold, 2600-fold, 2700-fold, 2800-fold, 2900-fold, 3000-fold, 3100-fold, 3200-fold, 3300-fold, 3400-fold, 3500-fold, 3600-fold, 3700-fold, 3800-fold, 3900-fold, 4000-fold, 4100-fold, 4200-fold, 4300-fold, 4400-fold, 4500-fold, 4600-fold, 4700-fold, 4800-fold, 4900-fold, 5000-fold, 5100-fold, 5200-fold, 5300-fold, 5400-fold, 5500-fold, 5600-fold, 5700-fold, 5800-fold, 5900-fold, or 6000-fold.

The vaccine dose can be between 1 μg to 10 mg active component/kg body weight/time and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

(1) Combinational Therapies with PD-1 and/or PD-L1 Antibodies

The present invention is also directed to a method of increasing an immune response in a mammal using the vaccine as described above. The vaccine as described above can comprise the cancer antigen and a PD1 antibody and/or PDL1 antibody as described above. The combination can be in a single formulation or can be separate and administered in sequence (either cancer antigen first and then PD1 antibody and/or PDL1 antibody, or PD1 antibody and/or PDL1 antibody first and then cancer antigen). In some embodiments, the cancer antigen can be administered to the subject about 30 seconds, 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 0.25 hours, 0.5 hours, 0.75 hours, 1 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, 96 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 1 week, 2 weeks, 3 weeks, 4 weeks, 5 weeks, 6 weeks, 7 weeks, or 8 weeks before the PD-1 antibody and/or PD-L1 antibody is administered to the subject. In other embodiments, the PD-1 antibody and/or PD-L1 antibody can be administered to the subject about 30 seconds, 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 0.25 hours, 0.5 hours, 0.75 hours, 1 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, 96 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 1 week, 2 weeks, 3 weeks, 4 weeks, 5 weeks, 6 weeks, 7 weeks, or 8 weeks before the cancer antigen is administered to the subject.

The combination of the cancer antigen and PD1 antibody and/or PDL1 antibody induces the immune system more efficiently than a vaccine comprising the cancer antigen alone. This more efficient immune response provides increased efficacy in the treatment and/or prevention of a particular cancer. Depending upon the antigen used in the vaccine combined with the PDL1 antibody or PD1 antibody, the treated cancer or tumor based growth can be any type of cancer such as, but not limited to, melanoma, blood cancers (e.g., leukemia, lymphoma, myeloma), lung carcinomas, esophageal squamous cell carcinomas, bladder cancer, colorectal cancer, esophagus, gastric cancer, hepatocarcinoma, head and neck, brain, anal cancer, non-small cell lung carcinoma, pancreatic cancer, synovial carcinoma, prostate cancer, testicular cancer, liver cancer, cervical cancer, recurrent respiratory papillomatosis, skin cancer and stomach cancer.

In some embodiments, the immune response can be increased by about 0.5-fold to about 15-fold, about 0.5-fold to about 10-fold, or about 0.5-fold to about 8-fold. Alternatively, the immune response in the subject administered the vaccine can be increased by at least about 0.5-fold, at least about 1.0-fold, at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, or at least about 15.0-fold.

In still other alternative embodiments, the immune response in the subject administered the vaccine can be increased about 50% to about 1500%, about 50% to about 1000%, or about 50% to about 800%. In other embodiments, the immune response in the subject administered the vaccine can be increased by at least about 50%, at least about 100%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1050%, at least about 1100%, at least about 1150%, at least about 1200%, at least about 1250%, at least about 1300%, at least about 1350%, at least about 1450%, or at least about 1500%.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

(2) Melanoma

The vaccine can be used to generate or elicit an immune response in a mammal that is reactive or directed to melanoma in the mammal or subject in need thereof. The elicited immune response can prevent melanoma growth. The elicited immune response can reduce melanoma growth. The elicited immune response can prevent and/or reduce metastasis of cancerous or tumor cells from a melanoma. Accordingly, the vaccine can be used in a method that treats and/or prevents melanoma in the mammal or subject administered the vaccine.

In some embodiments, the administered vaccine can mediate clearance or prevent growth of melanoma cells by inducing (1) humoral immunity via B cell responses to generate antibodies that block monocyte chemoattractant protein-1 (MCP-1) production, thereby retarding myeloid derived suppressor cells (MDSCs) and suppressing melanoma growth; (2) increase cytotoxic T lymphocyte such as CD8⁺ (CTL) to attack and kill melanoma cells; (3) increase T helper cell responses; and (4) increase inflammatory responses via IFN-γ and TFN-α or all of the aforementioned.

In some embodiments, the administered vaccine can increase melanoma free survival, reduce melanoma mass, increase melanoma survival, or a combination thereof in the subject. The administered vaccine can increase melanoma free survival by 30%, 31%, 32%, 33%, 34%, 35%, 36%, 37%, 38%, 39%, 40%, 41%, 42%, 43%, 44%, and 45% or more in the subject. The administered vaccine can reduce melanoma mass by 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, and 60% or more in the subject after immunization. The administered vaccine can prevent and block increases in monocyte chemoattractant protein 1 (MCP-1), a cytokine secreted by myeloid derived suppressor cells, in the subject. In some embodiments, the administered vaccine can prevent and block increases in MCP-1 within the melanoma tissue in the subject, thereby reducing vascularization of the melanoma tissue in the subject. The administered vaccine can increase melanoma survival by 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, and 60% or more in the subject.

### 8. ROUTES OF ADMINISTRATION

The vaccine or pharmaceutical composition can be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intrathecal, and intraarticular or combinations thereof. For veterinary use, the composition can be administered as a suitably acceptable formulation in accordance with normal veterinary practice. The veterinarian can readily determine the dosing regimen and route of administration that is most appropriate for a particular animal. The vaccine can be administered by traditional syringes, needleless injection devices, “microprojectile bombardment gene guns”, or other physical methods such as electroporation (“EP”), “hydrodynamic method”, or ultrasound.

The vector of the vaccine can be administering to the mammal by several well-known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The one or more cancer antigens of the vaccine can be administered via DNA injection and along with in vivo electroporation.

a. Electroporation

The vaccine or pharmaceutical composition can be administered by electroporation. Administration of the vaccine via electroporation can be accomplished using electroporation devices that can be configured to deliver to a desired tissue of a mammal a pulse of energy effective to cause reversible pores to form in cell membranes, and preferable the pulse of energy is a constant current similar to a preset current input by a user. The electroporation device can comprise an electroporation component and an electrode assembly or handle assembly. The electroporation component can include and incorporate one or more of the various elements of the electroporation devices, including: controller, current waveform generator, impedance tester, waveform logger, input element, status reporting element, communication port, memory component, power source, and power switch. The electroporation can be accomplished using an in vivo electroporation device, for example CELLECTRA® EP system (Inovio Pharmaceuticals, Inc., Blue Bell, Pa.) or Elgen electroporator (Inovio Pharmaceuticals, Inc.) to facilitate transfection of cells by the plasmid.

Examples of electroporation devices and electroporation methods that can facilitate administration of the DNA vaccines of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that can be used for facilitating administration of the DNA vaccines include those provided in U.S. patent application Ser. No. 11/874,072, filed Oct. 17, 2007, which claims the benefit under 35 USC 119(e) to U.S. Provisional Application Ser. Nos. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems can comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then administered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference in its entirety.

U.S. Patent Pub. 2005/0052630 submitted by Smith, et al. describes an electroporation device which can be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby fully incorporated by reference.

The electrode arrays and methods described in U.S. Pat. No. 7,245,963 and U.S. Patent Pub. 2005/0052630 can be adapted for deep penetration into not only tissues such as muscle, but also other tissues or organs. Because of the configuration of the electrode array, the injection needle is also inserted completely into the target organ, and the injection is administered perpendicular to the target issue, in the area that is pre-delineated by the electrodes. The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns administration of DNA using any of a variety of devices, and U.S. Pat. No. 7,328,064 issued Feb. 5, 2008, drawn to method of injecting DNA are contemplated herein. The above-mentioned patents are incorporated by reference in their entirety.

### 9. METHOD OF PREPARING THE VACCINE

Provided herein are methods for preparing the DNA plasmids that comprise the vaccines discussed herein. The DNA plasmids, after the final subcloning step into the mammalian expression plasmid, can be used to inoculate a cell culture in a large-scale fermentation tank, using known methods in the art.

The DNA plasmids for use with the EP devices of the present invention can be formulated or manufactured using a combination of known devices and techniques, but preferably they are manufactured using an optimized plasmid manufacturing technique that is described in a US published application no. 20090004716, which was filed on May 23, 2007. In some examples, the DNA plasmids used in these studies can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

The present invention has multiple aspects, illustrated by the following non-limiting examples.

### 10. EXAMPLES

### Example 1: Development of a Synthetic DNA Dog Tert Immune Therapeutic Vaccine

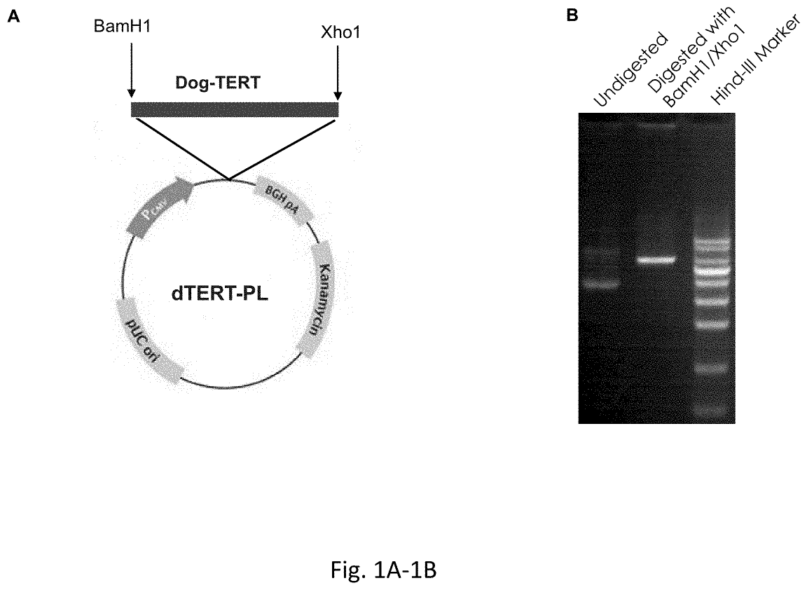

A synthetic consensus dog TERT (dTERT-PL) DNA vaccine was developed as a possible T cell immune therapy for canine B cell lymphoma (FIG. 1).

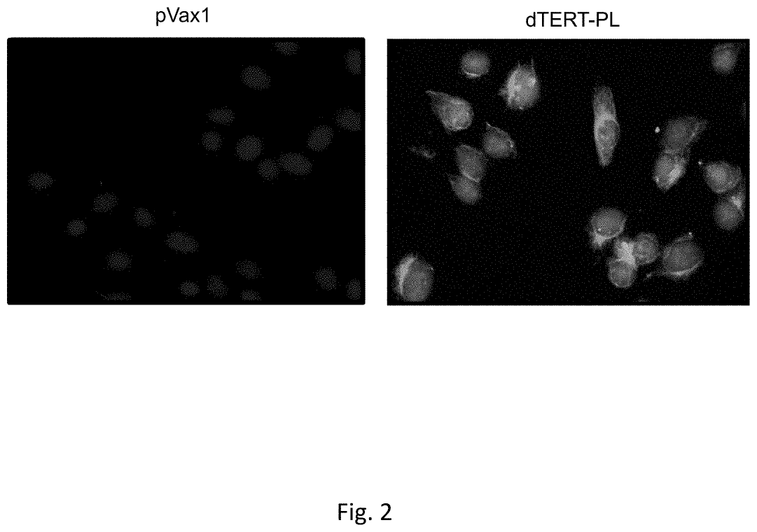

293T cells were transfected with pVax1 or Dog TERT-PL DNA construct (10 g). Two (2) days post transfection cells were fixed and stained with anti-TERT antibody for expression of TERT in transfected cells. A high level of expression of dTERT was observed in dTERT-PL transfected cells (FIG. 2).

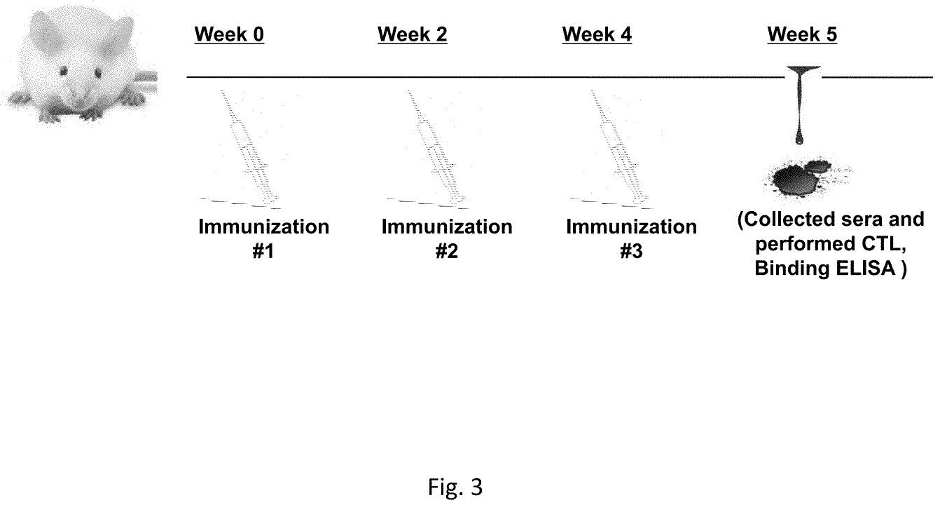

Mice (n=4 mice per group) were immunized with dTERT-PL according to the schedule shown in FIG. 3. Three immunizations were administered prior to sacrifice and sera collection. For these experiments, immunization was performed using electroporation.

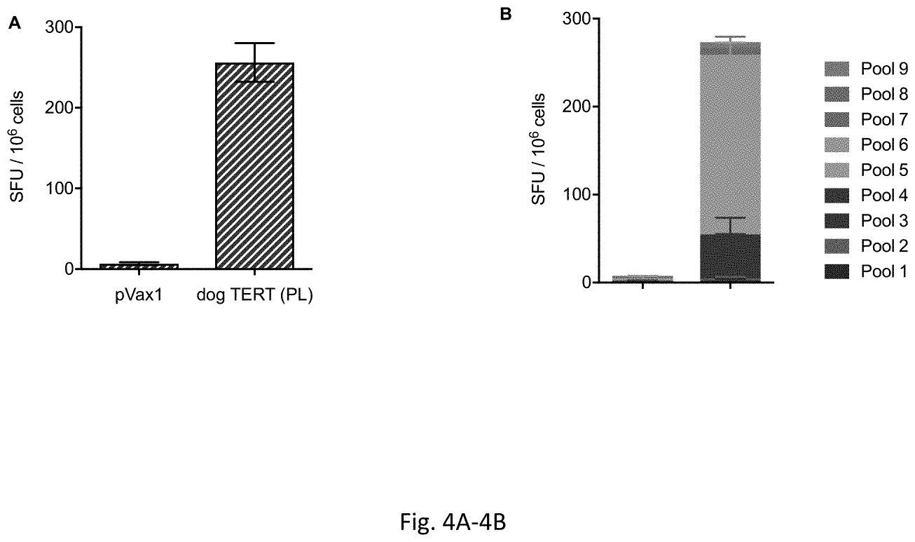

Cellular immune responses induced by dTERT were examined in C57BL/6 mice. Total dTERT-specific IFN-γ responses one week after the 3rd immunization with the dTERT vaccine (25 μg) are shown in FIG. 4A. FIG. 4B shows dTERT-specific IFN-γ responses for splenocytes from each mouse (4 mice per group) stimulated with dTERT peptide pools separately. The data suggests the long-term persistence of immune response after dTERT DNA vaccination.

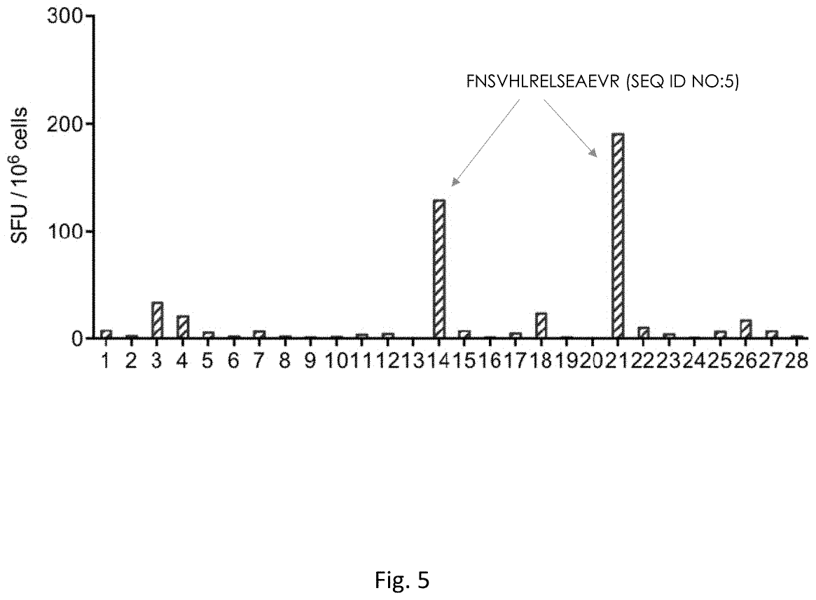

Dominant CTL epitope prediction by dTERT-PL DNA vaccine in C57/BL6 mice was performed. FIG. 5 shows that consensus-based dTERT DNA plasmid elicits significant cellular immune responses in mice after 3 vaccinations with electroporation, and a high level of IFN-γ⁺ T cells were observed with specific immunodominant and subdominant epitopes of dTERT in spleen.

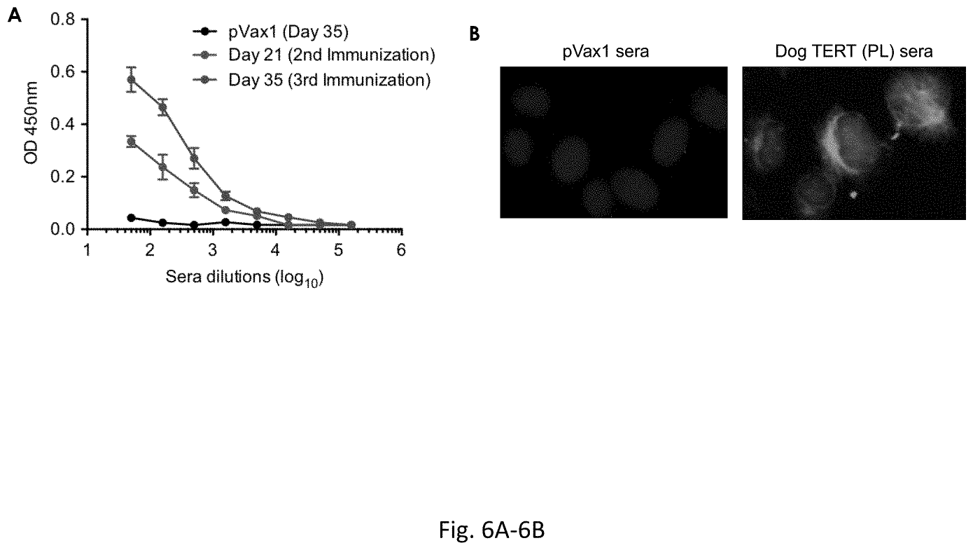

Total IgG antibody titers were measured in the sera of the immunized mice. Each group of mice (n=5) was immunized with 50 μg of dTERT-PL DNA. High anti-TERT total IgG levels were measured by ELISA in dTERT-PL vaccinated mice sera compared with pVax1 sera (FIG. 6A). Further, specificity was detected by immunofluorescence in 293T cells transfected with DNA plasmid vaccine encoding the dTERT and treated with immune serum from the mice (FIG. 6B).

In summary, consensus dTERT vaccines were developed encoding TERT genes and tested. dTERT vaccines are immunogenic in mice and elicited both a strong T cell and antibody immune response in vaccinated mice by intramuscular (i.m.) vaccination followed by electroporation. The humoral responses appear most potent to vaccinated mice and very specific to the target.

Characterization of CD8 epitope mapping shows reasonable levels of immune reactivity was induced by the dTERT vaccine in mice, and a dominant CD8 epitope was identified (SEQ ID NO:5).

The synthetic consensus sequence therefore is a potential candidate as a possible T cell immune therapy for canine B cell lymphoma vaccine development.

### Example 2: Sequences of a Synthetic Consensus dTERT

SEQ ID NO:1, synthetic consensus dTERT nucleotide sequence

SEQ ID NO:2, synthetic consensus dTERT amino acid sequence

SEQ ID NO:3, synthetic consensus dTERT nucleotide sequence operably linked to an IgE leader sequence

SEQ ID NO:4, synthetic consensus dTERT amino acid sequence operably linked to an IgE leader sequence

SEQ ID NO:5, immunodominant epitope of synthetic consensus dTERT

SEQ ID NO:6, immunoglobulin E (IgE) leader, nucleotide sequence

SEQ ID NO:7, immunoglobulin E (IgE) leader, amino acid sequence

What is claimed is:

1. A vaccine comprising a nucleotide sequence encoding a consensus dTERT antigen, wherein the consensus dTERT antigen comprises an amino acid sequence selected from the group consisting of SEQ ID NO:2, SEQ ID NO:4, an amino acid sequence that is 95% identical or greater to SEQ ID NO:2 and an amino acid sequence that is 95% identical or greater to SEQ ID NO:4.

2. The vaccine of claim 1, wherein the vaccine further comprises one or more nucleotide sequences encoding one or more additional cancer antigens.

3. The vaccine of claim 2, wherein the one or more additional cancer antigens comprise one or more antigens selected from the group consisting of the amino acid sequence of tyrosinase (Tyr), the amino acid sequence of tyrosinase-related protein 1 (TYRP1), the amino acid sequence of tyrosinase-related protein 2 (TYRP2), the amino acid sequence of melanoma-associated antigen 4 protein (MAGEA4), the amino acid sequence of growth hormone release hormone (GHRH), the amino acid sequence of MART-1/melan-A antigen (MART-1/Melan-A), the amino acid sequence of cancer testis antigen (NY-ESO-1), the amino acid sequence of cancer testis antigen II (NY-ESO-2), the amino acid sequence of PRAME, the amino acid sequence of WT1, the amino acid sequence of PSA, the amino acid sequence of PSMA, the amino acid sequence of STEAP, the amino acid sequence of PSCA, the amino acid sequence of MAGE A1, the amino acid sequence of gp100, the amino acid sequence of a viral antigen, and a combination thereof.

4. The vaccine of claim 1, wherein the vaccine further comprises one or more nucleotide sequences encoding one or more immune checkpoint inhibitors.

5. The vaccine of claim 4, wherein the immune checkpoint inhibitor is selected from the group consisting of: anti-PD-1 antibody, anti-PD-L1 antibody, anti-TIM-3 antibody, anti-LAG-3 antibody, anti-CTLA4 antibody, and a combination thereof.

6. The vaccine of claim 1, wherein the nucleotide sequence comprises a nucleotide sequence selected from the group consisting of: SEQ ID NO:1, SEQ ID NO:3, a nucleotide sequence that is 95% identical or greater to SEQ ID NO:1, and a nucleotide sequence that is 95% identical or greater to SEQ ID NO:3.

7. The vaccine of claim 1, wherein the nucleotide sequence comprises one or more plasmids.

8. The vaccine of claim 1, further comprising a nucleotide sequence encoding an adjuvant.

9. The vaccine of claim 8, wherein the adjuvant is IL-12, IL-15, IL-28, or RANTES.

10. A method of treating cancer in a subject in need thereof, the method comprising administering the vaccine of claim 1 to the subject.

11. The method of claim 10, wherein the administering step comprises electroporation.

12. The method of claim 10, further comprising administering one or more nucleotide sequences encoding one or more immune checkpoint inhibitors.

13. The method of claim 12, wherein the immune checkpoint inhibitor is selected from the group consisting of: anti-PD-1 antibody, anti-PD-L1 antibody, anti-TIM-3 antibody, anti-LAG-3 antibody, anti-CTLA4 antibody, and a combination thereof.

14. The method of claim 10, wherein the cancer is selected from the group consisting of: a blood cancer, melanoma, head and neck cancer, prostate cancer, liver cancer, cervical cancer, anal cancer, a papilloma and a combination thereof.

15. A nucleic acid molecule comprising one or more nucleotide sequences selected from the group consisting of: SEQ ID NO:1, SEQ ID NO:3, a nucleotide sequence that is 95% identical or greater to SEQ ID NO:1, and a nucleotide sequence that is 95% identical or greater to SEQ ID NO:3.

16. The nucleic acid molecule of claim 15, wherein the nucleotide sequence comprises one or more plasmids.

17. A protein comprising one or more amino acid sequences selected from the group consisting of: SEQ ID NO:2, SEQ ID NO:4, an amino acid sequence that is 95% identical or greater to SEQ ID NO:2, and an amino acid sequence that is 95% identical or greater to SEQ ID NO:4.
